SEQUENCE LISTING (171 sequences)

SEQ ID NO: 1 - protein, 5 aa, Artificial Sequence
  Synthetic polypeptide
SYGMH

SEQ ID NO: 2 - protein, 17 aa, Artificial Sequence
  Synthetic polypeptide
VISYEGSNKYYAESVKG

SEQ ID NO: 3 - protein, 13 aa, Artificial Sequence
  Synthetic polypeptide
DRGTIFGNYGLEV

SEQ ID NO: 4 - protein, 122 aa, Artificial Sequence
  Synthetic polypeptide
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 5 - protein, 16 aa, Artificial Sequence
  Synthetic polypeptide
RSSQSLLHKNAFNYLD

SEQ ID NO: 6 - protein, 7 aa, Artificial Sequence
  Synthetic polypeptide
LGSNRAS

SEQ ID NO: 7 - protein, 9 aa, Artificial Sequence
  Synthetic polypeptide
MQALQTPFT

SEQ ID NO: 8 - protein, 112 aa, Artificial Sequence
  Synthetic polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 9 - protein, 249 aa, Artificial Sequence
  Synthetic polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 10 - protein, 991 aa, Artificial Sequence
  Synthetic polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 5 aa, Artificial Sequence
  Synthetic polypeptide
NYGMN

SEQ ID NO: 12 - protein, 17 aa, Artificial Sequence
  Synthetic polypeptide
WINTYTGEPTYADKFQG

SEQ ID NO: 13 - protein, 13 aa, Artificial Sequence
  Synthetic polypeptide
WSWSDGYYVYFDY

SEQ ID NO: 14 - protein, 122 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 15 - protein, 17 aa, Artificial Sequence
  Synthetic polypeptide
KSSQSVLDSSTNKNSLA

SEQ ID NO: 16 - protein, 7 aa, Artificial Sequence
  Synthetic polypeptide
WASTRES

SEQ ID NO: 17 - protein, 9 aa, Artificial Sequence
  Synthetic polypeptide
QQSAHFPIT

SEQ ID NO: 18 - protein, 113 aa, Artificial Sequence
  Synthetic polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 19 - protein, 250 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 20 - protein, 993 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 7 aa, Artificial Sequence
  Synthetic polypeptide
NARMGVS

SEQ ID NO: 22 - protein, 16 aa, Artificial Sequence
  Synthetic polypeptide
HIFSNDEKSYSTSLKN

SEQ ID NO: 23 - protein, 14 aa, Artificial Sequence
  Synthetic polypeptide
IVGYGSGWYGFFDY

SEQ ID NO: 24 - protein, 124 aa, Artificial Sequence
  Synthetic polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 25 - protein, 11 aa, Artificial Sequence
  Synthetic polypeptide
RASQGIRNDLG

SEQ ID NO: 26 - protein, 7 aa, Artificial Sequence
  Synthetic polypeptide
AASTLQS

SEQ ID NO: 27 - protein, 9 aa, Artificial Sequence
  Synthetic polypeptide
LQHNSYPLT

SEQ ID NO: 28 - protein, 107 aa, Artificial Sequence
  Synthetic polypeptide
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 29 - protein, 246 aa, Artificial Sequence
  Synthetic polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 30 - protein, 989 aa, Artificial Sequence
  Synthetic polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 5 aa, Artificial Sequence
  Synthetic polypeptide
SYYWS

SEQ ID NO: 32 - protein, 16 aa, Artificial Sequence
  Synthetic polypeptide
YVYYSGTTNYNPSLKS

SEQ ID NO: 33 - protein, 10 aa, Artificial Sequence
  Synthetic polypeptide
IAVTGFYFDY

SEQ ID NO: 34 - protein, 118 aa, Artificial Sequence
  Synthetic polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 35 - protein, 12 aa, Artificial Sequence
  Synthetic polypeptide
RASQRVNNNYLA

SEQ ID NO: 36 - protein, 7 aa, Artificial Sequence
  Synthetic polypeptide
GASSRAT

SEQ ID NO: 37 - protein, 9 aa, Artificial Sequence
  Synthetic polypeptide
QQYDRSPLT

SEQ ID NO: 38 - protein, 108 aa, Artificial Sequence
  Synthetic polypeptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 39 - protein, 241 aa, Artificial Sequence
  Synthetic polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 40 - protein, 982 aa, Artificial Sequence
  Synthetic polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 5 aa, Artificial Sequence
  Synthetic polypeptide
NHIIH

SEQ ID NO: 42 - protein, 17 aa, Artificial Sequence
  Synthetic polypeptide
YINPYPGYHAYNEKFQG

SEQ ID NO: 43 - protein, 12 aa, Artificial Sequence
  Synthetic polypeptide
DGYYRDTDVLDY

SEQ ID NO: 44 - protein, 121 aa, Artificial Sequence
  Synthetic polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 45 - protein, 11 aa, Artificial Sequence
  Synthetic polypeptide
QASQDISNYLN

SEQ ID NO: 46 - protein, 7 aa, Artificial Sequence
  Synthetic polypeptide
YTSRLHT

SEQ ID NO: 47 - protein, 9 aa, Artificial Sequence
  Synthetic polypeptide
QQGNTLPWT

SEQ ID NO: 48 - protein, 107 aa, Artificial Sequence
  Synthetic polypeptide
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIK

SEQ ID NO: 49 - protein, 243 aa, Artificial Sequence
  Synthetic polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIK

SEQ ID NO: 50 - protein, 986 aa, Artificial Sequence
  Synthetic polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 5 aa, Artificial Sequence
  Synthetic polypeptide
DYYMY

SEQ ID NO: 52 - protein, 17 aa, Artificial Sequence
  Synthetic polypeptide
IISDGGYYTYYSDIIKG

SEQ ID NO: 53 - protein, 12 aa, Artificial Sequence
  Synthetic polypeptide
GFPLLRHGAMDY

SEQ ID NO: 54 - protein, 121 aa, Artificial Sequence
  Synthetic polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSS

SEQ ID NO: 55 - protein, 11 aa, Artificial Sequence
  Synthetic polypeptide
KASQNVDTNVA

SEQ ID NO: 56 - protein, 7 aa, Artificial Sequence
  Synthetic polypeptide
SASYVYW

SEQ ID NO: 57 - protein, 9 aa, Artificial Sequence
  Synthetic polypeptide
QQYDQQLIT

SEQ ID NO: 58 - protein, 107 aa, Artificial Sequence
  Synthetic polypeptide
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 59 - protein, 243 aa, Artificial Sequence
  Synthetic polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 60 - protein, 986 aa, Artificial Sequence
  Synthetic polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 62 - protein, 5 aa, Artificial Sequence
  Synthetic polypeptide
IYAMN

SEQ ID NO: 63 - protein, 19 aa, Artificial Sequence
  Synthetic polypeptide
RIRSKYNNYATYYADSVKS

SEQ ID NO: 64 - protein, 14 aa, Artificial Sequence
  Synthetic polypeptide
HGNFGNSYVSFFAY

SEQ ID NO: 65 - protein, 5 aa, Artificial Sequence
  Synthetic polypeptide
KYAMN

SEQ ID NO: 66 - protein, 19 aa, Artificial Sequence
  Synthetic polypeptide
RIRSKYNNYATYYADSVKD

SEQ ID NO: 67 - protein, 14 aa, Artificial Sequence
  Synthetic polypeptide
HGNFGNSYISYWAY

SEQ ID NO: 68 - protein, 5 aa, Artificial Sequence
  Synthetic polypeptide
SYAMN

SEQ ID NO: 69 - protein, 19 aa, Artificial Sequence
  Synthetic polypeptide
RIRSKYNNYATYYADSVKG

SEQ ID NO: 70 - protein, 14 aa, Artificial Sequence
  Synthetic polypeptide
HGNFGNSYLSFWAY

SEQ ID NO: 71 - protein, 5 aa, Artificial Sequence
  Synthetic polypeptide
RYAMN

SEQ ID NO: 72 - protein, 14 aa, Artificial Sequence
  Synthetic polypeptide
HGNFGNSYLSYFAY

SEQ ID NO: 73 - protein, 14 aa, Artificial Sequence
  Synthetic polypeptide
HGNFGNSYTSYYAY

SEQ ID NO: 74 - protein, 5 aa, Artificial Sequence
  Synthetic polypeptide
VYAMN

SEQ ID NO: 75 - protein, 19 aa, Artificial Sequence
  Synthetic polypeptide
RIRSKYNNYATYYADSVKK

SEQ ID NO: 76 - protein, 14 aa, Artificial Sequence
  Synthetic polypeptide
HGNFGNSYISWWAY

SEQ ID NO: 77 - protein, 14 aa, Artificial Sequence
  Synthetic polypeptide
HGNFGNSYLSWWAY

SEQ ID NO: 78 - protein, 5 aa, Artificial Sequence
  Synthetic polypeptide
GYAMN

SEQ ID NO: 79 - protein, 19 aa, Artificial Sequence
  Synthetic polypeptide
RIRSKYNNYATYYADSVKE

SEQ ID NO: 80 - protein, 14 aa, Artificial Sequence
  Synthetic polypeptide
HRNFGNSYLSWFAY

SEQ ID NO: 81 - protein, 14 aa, Artificial Sequence
  Synthetic polypeptide
HGNFGNSYVSWWAY

SEQ ID NO: 82 - protein, 14 aa, Artificial Sequence
  Synthetic polypeptide
GSSTGAVTSGYYPN

SEQ ID NO: 83 - protein, 7 aa, Artificial Sequence
  Synthetic polypeptide
GTKFLAP

SEQ ID NO: 84 - protein, 9 aa, Artificial Sequence
  Synthetic polypeptide
ALWYSNRWV

SEQ ID NO: 85 - protein, 14 aa, Artificial Sequence
  Synthetic polypeptide
RSSTGAVTSGYYPN

SEQ ID NO: 86 - protein, 7 aa, Artificial Sequence
  Synthetic polypeptide
ATDMRPS

SEQ ID NO: 87 - protein, 14 aa, Artificial Sequence
  Synthetic polypeptide
GSSTGAVTSGNYPN

SEQ ID NO: 88 - protein, 9 aa, Artificial Sequence
  Synthetic polypeptide
VLWYSNRWV

SEQ ID NO: 89 - protein, 125 aa, Artificial Sequence
  Synthetic polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 90 - protein, 125 aa, Artificial Sequence
  Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 91 - protein, 125 aa, Artificial Sequence
  Synthetic polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 92 - protein, 125 aa, Artificial Sequence
  Synthetic polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 93 - protein, 125 aa, Artificial Sequence
  Synthetic polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 94 - protein, 125 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 95 - protein, 125 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 96 - protein, 125 aa, Artificial Sequence
  Synthetic polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 97 - protein, 125 aa, Artificial Sequence
  Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 98 - protein, 109 aa, Artificial Sequence
  Synthetic polypeptide
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 99 - protein, 109 aa, Artificial Sequence
  Synthetic polypeptide
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 100 - protein, 109 aa, Artificial Sequence
  Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 101 - protein, 249 aa, Artificial Sequence
  Synthetic polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 102 - protein, 249 aa, Artificial Sequence
  Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 103 - protein, 249 aa, Artificial Sequence
  Synthetic polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 104 - protein, 249 aa, Artificial Sequence
  Synthetic polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 105 - protein, 249 aa, Artificial Sequence
  Synthetic polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 106 - protein, 249 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 107 - protein, 249 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 108 - protein, 249 aa, Artificial Sequence
  Synthetic polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 109 - protein, 249 aa, Artificial Sequence
  Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 249 aa, Artificial Sequence
  Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 111 - protein, 4 aa, Artificial Sequence
  Synthetic polypeptide
GGGG

SEQ ID NO: 112 - protein, 5 aa, Artificial Sequence
  Synthetic polypeptide
GGGGS

SEQ ID NO: 113 - protein, 6 aa, Artificial Sequence
  Synthetic polypeptide
PGGGGS

SEQ ID NO: 114 - protein, 6 aa, Artificial Sequence
  Synthetic polypeptide
PGGDGS

SEQ ID NO: 115 - protein, 6 aa, Artificial Sequence
  Synthetic polypeptide
SGGGGS

SEQ ID NO: 116 - protein, 9 aa, Artificial Sequence
  Synthetic polypeptide
GGGGSGGGS

SEQ ID NO: 117 - protein, 5 aa, Artificial Sequence
  Synthetic polypeptide
GGGGQ

SEQ ID NO: 118 - protein, 10 aa, Artificial Sequence
  Synthetic polypeptide
GGGGSGGGGS

SEQ ID NO: 119 - protein, 15 aa, Artificial Sequence
  Synthetic polypeptide
GGGGSGGGGSGGGGS

SEQ ID NO: 120 - protein, 20 aa, Artificial Sequence
  Synthetic polypeptide
GGGGSGGGGSGGGGSGGGGS

SEQ ID NO: 121 - protein, 25 aa, Artificial Sequence
  Synthetic polypeptide
GGGGSGGGGSGGGGSGGGGSGGGGS

SEQ ID NO: 122 - protein, 30 aa, Artificial Sequence
  Synthetic polypeptide
GGGGSGGGGSGGGGSGGGGSGGGGSGGGGS

SEQ ID NO: 123 - protein, 35 aa, Artificial Sequence
  Synthetic polypeptide
GGGGSGGGGSGGGGSGGGGSGGGGSGGGGSGGGGS

SEQ ID NO: 124 - protein, 40 aa, Artificial Sequence
  Synthetic polypeptide
GGGGSGGGGSGGGGSGGGGSGGGGSGGGGSGGGGSGGGGS

SEQ ID NO: 125 - protein, 10 aa, Artificial Sequence
  Synthetic polypeptide
DKTHTCPPCP

SEQ ID NO: 126 - protein, 15 aa, Artificial Sequence
  Synthetic polypeptide
EPKSCDKTHTCPPCP

SEQ ID NO: 127 - protein, 12 aa, Artificial Sequence
  Synthetic polypeptide
ERKCCVECPPCP

SEQ ID NO: 128 - protein, 16 aa, Artificial Sequence
  Synthetic polypeptide
ELKTPLDTTHTCPRCP

SEQ ID NO: 129 - protein, 15 aa, Artificial Sequence
  Synthetic polypeptide
EPKSCDTPPPCPRCP

SEQ ID NO: 130 - protein, 17 aa, Artificial Sequence
  Synthetic polypeptide
ELKTPLGDTTHTCPRCP

SEQ ID NO: 131 - protein, 12 aa, Artificial Sequence
  Synthetic polypeptide
ESKYGPPCPSCP

SEQ ID NO: 132 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 134 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 136 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 138 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 140 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 142 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 144 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 146 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 148 - protein, 482 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 5 aa, Artificial Sequence
  Synthetic polypeptide
GYYMH

SEQ ID NO: 150 - protein, 17 aa, Artificial Sequence
  Synthetic polypeptide
WINPNSGGTKYAQKFQG

SEQ ID NO: 151 - protein, 16 aa, Artificial Sequence
  Synthetic polypeptide
DRITVAGTYYYYGMDV

SEQ ID NO: 152 - protein, 125 aa, Artificial Sequence
  Synthetic polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 153 - protein, 125 aa, Artificial Sequence
  Synthetic polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 154 - protein, 11 aa, Artificial Sequence
  Synthetic polypeptide
RASQGVNNWLA

SEQ ID NO: 155 - protein, 7 aa, Artificial Sequence
  Synthetic polypeptide
TASSLQS

SEQ ID NO: 156 - protein, 9 aa, Artificial Sequence
  Synthetic polypeptide
QQANSFPIT

SEQ ID NO: 157 - protein, 107 aa, Artificial Sequence
  Synthetic polypeptide
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 158 - protein, 247 aa, Artificial Sequence
  Synthetic polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 159 - protein, 247 aa, Artificial Sequence
  Synthetic polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 160 - protein, 990 aa, Artificial Sequence
  Synthetic polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 990 aa, Artificial Sequence
  Synthetic polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 5 aa, Artificial Sequence
  Synthetic polypeptide
GYYWS

SEQ ID NO: 163 - protein, 16 aa, Artificial Sequence
  Synthetic polypeptide
DIDASGSTKYNPSLKS

SEQ ID NO: 164 - protein, 12 aa, Artificial Sequence
  Synthetic polypeptide
KKYSTVWSYFDN

SEQ ID NO: 165 - protein, 120 aa, Artificial Sequence
  Synthetic polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 166 - protein, 11 aa, Artificial Sequence
  Synthetic polypeptide
SGDKLGDKYAS

SEQ ID NO: 167 - protein, 7 aa, Artificial Sequence
  Synthetic polypeptide
QDRKRPS

SEQ ID NO: 168 - protein, 9 aa, Artificial Sequence
  Synthetic polypeptide
QAWGSSTAV

SEQ ID NO: 169 - protein, 106 aa, Artificial Sequence
  Synthetic polypeptide
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 170 - protein, 241 aa, Artificial Sequence
  Synthetic polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 171 - protein, 984 aa, Artificial Sequence
  Synthetic polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0398147 A1
(43) Pub. Date: Dec. 14, 2023

(54) METHODS FOR ADMINISTERING THERAPEUTIC DOSES OF BISPECIFIC T-CELL ENGAGING MOLECULES FOR THE TREATMENT OF CANCER

(71) Applicants: AMGEN INC., Thousand Oaks, CA (US); AMGEN RESEARCH (MUNICH) GmbH, Munich (DE)

(72) Inventors: Adam B. MCCULLOUGH, Westlake Village, CA (US); Hosein KOUROS-MEHR, Los Angeles, CA (US); Peter KUFER, Munich (DE); Mark SALVATI, Westlake Village, CA (US); Alexander C. MINELLA, Westlake Village, CA (US); Dirk NAGORSEN, Thousand Oaks, CA (US); Vijay UPRETI, South San Francisco, CA (US); Mukul MINOCHA, Oak Park, CA (US); Brett HOUK, Newbury Park, CA (US)

(73) Assignees: AMGEN INC., Thousand Oaks, CA (US); AMGEN RESEARCH (MUNICH) GmbH, Munich (DE)

(21) Appl. No.: 18/026,505

(22) PCT Filed: Sep. 15, 2021
(86) PCT No.: PCT/US2021/050546

§ 371 Date: Mar. 15, 2023

Related U.S. Application Data
(60) Provisional application No. 63/079,418, filed on Sep. 16, 2020.

(51) Int. Cl. A61K 35/17; C07K 16/28; A61P 35/00; C07K 14/81; A61K 39/00

(52) U.S. Cl. CPC A61K 35/17; C07K 16/2809; A61P 35/00; C07K 14/8103; A61K 39/4611; C12Y 304/17021; A61K 2239/13; A61K 2239/58; A61K 2239/38; A61K 2239/31

(57) ABSTRACT

The present invention relates to methods for administering therapeutic doses of bispecific T-cell engaging molecules for the treatment of cancer in a patient. The administration methods reduce the incidence and/or severity of adverse events, such as cytokine release syndrome, and entail administering to a patient a priming dose of the bispecific T-cell engaging molecule by continuous intravenous infusion over a period of days followed by administration of a therapeutic dose of the bispecific T-cell engaging molecule by a bolus intravenous infusion at dosing intervals of at least a week.

### CROSS-REFERENCE TO RELATED APPLICATIONS

This application claims the benefit of U.S. Provisional Application No. 63/079,418, filed Sep. 16, 2020, which is hereby incorporated by reference in its entirety.

### DESCRIPTION OF THE TEXT FILE SUBMITTED ELECTRONICALLY

The present application contains a Sequence Listing, which has been submitted electronically in ASCII format and is hereby incorporated by reference in its entirety. The computer readable format copy of the Sequence Listing, which was created on Sep. 13, 2021, is named A-2684-WO-PCT_ST25 and is 222 kilobytes in size.

### FIELD OF THE INVENTION

The present invention relates to the fields of immuno-oncology and biopharmaceuticals. In particular, the invention relates to methods for administering therapeutic doses of a bispecific T-cell engaging molecule, which specifically binds to a target cancer cell antigen and cluster of differentiation 3 (CD3), for the treatment of cancer in a patient in need thereof. The methods employ specific administration regimens that reduce the incidence and/or severity of adverse events, such as cytokine release syndrome, in patients undergoing treatment for cancer.

### BACKGROUND OF THE INVENTION

Bispecific T-cell engaging molecules are new immunotherapies being developed for the treatment of various cancers. These molecules typically have at least one binding domain that is specific for a cell-surface antigen expressed on cancer cells and at least another binding domain that is specific for CD3, a subunit of the T cell receptor complex expressed on T cells. Bispecific T cell engaging molecules are designed to connect T cells with target cancer cells and potently activate the inherent cytolytic potential of T cells against the target cancer cells. The first generation of bispecific T cell engaging molecules (see, e.g., WO 99/54440, WO 2005/040220, and WO 2008/119567) are typically administered by continuous intravenous infusion due to half-lives of less than a day. A second generation of bispecific T cell engaging molecules (see, e.g., WO 2013/128027, WO 2014140358, WO 2014/144722, WO 2014/151910, and WO 2017/134140) have been designed, at least in part, to increase the serum half-life of the molecules to enable dosing paradigms that allow for administration at intermittent dosing intervals.

Because the mechanism of action of bispecific T cell engaging molecules involves T cell activation, a potential side effect of these molecules is cytokine release syndrome (CRS). CRS can occur when large numbers of T cells are activated and release inflammatory cytokines. Symptoms of CRS can range from mild, flu-like symptoms, such as fever, fatigue, headache, and rash, to severe life-threatening consequences of an excessive inflammatory response (Shimabukuro-Vornhagen et al., Journal for ImmunoTherapy of Cancer, Vol. 6: 56, 2018). More severe cases of CRS are characterized by hypotension and symptoms of acute respiratory distress that can progress to vasopressor-requiring circulatory shock, vascular leakage, and multi-organ system failure (Shimabukuro-Vornhagen et al., 2018, supra). These side effects may be attributed in part to the pharmacokinetic profile (higher peak serum levels) of these bispecific T-cell engaging molecules especially when administered as a short-term infusion (e.g. over 1 hour) at the time of treatment initiation. To minimize the effects of cytokine elevation and the development of CRS, bispecific T cell engaging molecules can be administered at lower doses or by employing anti-histamines or corticosteroid pre-treatments (Topp et al., Lancet Oncol., Vol. 16: 57-66, 2015). In addition, tocilizumab, an IL-6 receptor antibody, has been used prophylactically or therapeutically to prevent or treat symptoms of CRS in patients receiving immunotherapies (see, e.g., Maude et al., Cancer J., Vol. 20:119-122, 2014). However, these different approaches to managing CRS have various levels of effectiveness depending on the type of immunotherapy employed and characteristics of the patient to be treated. Moreover, some of these mitigation approaches can affect the efficacy of the immunotherapy.

Thus, there remains a need in the art for strategies to effectively manage the occurrence or severity of CRS and other adverse events associated with bispecific T-cell engaging immunotherapy while maximizing the therapeutic benefit of such immunotherapies in patients with cancer.

### SUMMARY OF THE INVENTION

The present invention is based, in part, on the design of administration regimens for bispecific T-cell engaging molecules, particularly bispecific T-cell engaging molecules with extended half-lives, that deliver therapeutic doses as early as possible in the first cycle of treatment while reducing the number and severity of adverse events, particularly CRS events, in a patient diagnosed with cancer. Accordingly, in certain embodiments, the present invention provides methods for administering a therapeutic dose of a bispecific T-cell engaging molecule to a patient diagnosed with cancer, comprising administering to the patient an initiation cycle of the bispecific T-cell engaging molecule, said initiation cycle comprising: administering a priming dose of the bispecific T-cell engaging molecule by continuous intravenous infusion over a period of time; and administering after the priming dose a therapeutic dose of the bispecific T-cell engaging molecule by a bolus intravenous infusion or subcutaneous injection.

In certain embodiments of the methods of the invention, the initiation cycle comprises administering a priming dose of the bispecific T-cell engaging molecule by continuous intravenous (IV) infusion (also referred to as extended IV infusion (eIV)) over a period of at least 1 day, for example over a period of 1 day to 7 days. Administration of the first dose (i.e. priming dose) of the bispecific T-cell engaging molecule by a continuous IV infusion over such an extended period of time avoids rapid increases in peak serum concentrations of the molecule, which has been observed to be associated with the incidence and grade of CRS in patients. Without being bound by any particular theory, it is believed that administration of the priming dose by a continuous IV infusion over an extended period of time will decrease and delay peak serum concentrations of the molecule, thereby reducing the frequency and severity of CRS and other adverse events. In some embodiments, the priming dose of the bispecific T-cell engaging molecule is administered by continuous IV infusion over a period of about 2 days. In other embodiments, the priming dose of the bispecific T-cell engaging molecule is administered by continuous IV infusion over a period of about 3 days. In one embodiment, the priming dose of the bispecific T-cell engaging molecule is administered by continuous IV infusion over a period of about 4 days. In another embodiment, the priming dose of the bispecific T-cell engaging molecule is administered by continuous IV infusion over a period of about 5 days. In yet another embodiment, the priming dose of the bispecific T-cell engaging molecule is administered by continuous IV infusion over a period of about 7 days. The continuous IV infusion may be given either using a constant flow rate such that the continuous IV infusion delivers the priming dose at a constant rate (e.g. fixed dose per day) or at a variable flow rate such that the continuous IV infusion delivers the priming dose at a variable rate (e.g. increasing dose each day) over the period of the infusion.

In some embodiments of the methods of the invention, the initiation cycle comprises administering a therapeutic dose of the bispecific T-cell engaging molecule by a bolus IV infusion after administration of the priming dose (e.g. after completion of the continuous infusion period). The therapeutic dose may be administered on the same day (e.g. within 30 min to 18 hours) following completion of the continuous IV infusion of the priming dose or 1 day (e.g. the next day) following completion of the continuous IV infusion of the priming dose. Alternatively, the administration of the therapeutic dose may be delayed by two or more days following completion of the continuous IV infusion of the priming dose. In certain embodiments, the therapeutic dose is administered about 3 days, about 4 days, about 5 days, about 6 days, or about 7 days after administration of the priming dose (e.g. after completion of the continuous infusion period). In some embodiments of the methods of the invention, the initiation cycle further comprises administering a boost dose of the bispecific T-cell engaging molecule by a bolus intravenous infusion after administration of the priming dose and before the administration of the therapeutic dose. In such embodiments, the boost day may be administered 1 day (e.g. next day) following completion of the continuous IV infusion of the priming dose and at least 2 days, 3 days, 4 days, 5 days, or 6 days before the administration of the therapeutic dose. In any of the foregoing embodiments, the bolus IV infusion of the therapeutic dose and/or the boost dose is an infusion of less than 3 hours and is typically an infusion of about 30 minutes to about 90 minutes. In certain embodiments, the bolus IV infusion is an infusion of about 60 minutes. In other embodiments of the methods of the invention, the therapeutic dose and/or the boost dose of the bispecific T-cell engaging molecule can be administered as a subcutaneous injection.

In certain embodiments of the methods of the invention, following the first administration of the therapeutic dose of the bispecific T-cell engaging molecule in the initiation cycle, the therapeutic dose can be administered by a bolus IV infusion or a subcutaneous injection at a dosing interval of at least 7 days for the duration of the initiation cycle. For example, in one embodiment, the therapeutic dose of the bispecific T-cell engaging molecule is subsequently administered by a bolus IV infusion once every 7 days (e.g. weekly) for the duration of the initiation cycle. In another embodiment, the therapeutic dose of the bispecific T-cell engaging molecule is subsequently administered by a bolus IV infusion once every 14 days (e.g. biweekly) for the duration of the initiation cycle. In any such embodiments, the duration of the initiation cycle can be about 28 days.

In some embodiments of the methods of the invention, the initiation cycle is about 28 days and comprises administering a priming dose of the bispecific T-cell engaging molecule by continuous IV infusion over days 1 to 3 of the cycle and administering a therapeutic dose of the bispecific T-cell engaging molecule by bolus IV infusion on days 8 and 22 of the cycle. In other embodiments of the methods of the invention, the initiation cycle is about 28 days and comprises administering a priming dose of the bispecific T-cell engaging molecule by continuous IV infusion over days 1 to 4 of the cycle and administering a therapeutic dose of the bispecific T-cell engaging molecule by bolus IV infusion on days 8, 15, and 22 of the cycle. In some embodiments of the methods of the invention, the initiation cycle is about 28 days and comprises administering a priming dose of the bispecific T-cell engaging molecule by continuous IV infusion over days 1 to 5 of the cycle and administering a therapeutic dose of the bispecific T-cell engaging molecule by bolus IV infusion on days 8 and 22 of the cycle. In still other embodiments of the methods of the invention, the initiation cycle is about 28 days and comprises administering a priming dose of the bispecific T-cell engaging molecule by continuous IV infusion over days 1 to 7 of the cycle and administering a therapeutic dose of the bispecific T-cell engaging molecule by bolus IV infusion on days 8, 15, and 22 of the cycle. In yet other embodiments of the methods of the invention, the initiation cycle is about 28 days and comprises administering a priming dose of the bispecific T-cell engaging molecule by continuous IV infusion over days 1 to 2 of the cycle and administering a therapeutic dose of the bispecific T-cell engaging molecule by bolus IV infusion on days 8, 15, and 22 of the cycle. In one such embodiment, the initiation cycle may further comprise administering a boost dose of the bispecific T-cell engaging molecule by bolus IV infusion on day 3 of the cycle.

The therapeutic doses of the bispecific T-cell engaging molecule administered according to the methods of the invention may range from about 50 μg to about 200 mg or from about 200 μg to about 80 mg depending on the specific bispecific T-cell engaging molecule employed and the type, grade, or stage of cancer to be treated in the patient. In some embodiments, suitable therapeutic doses of a PSMA×CD3 bispecific T-cell engaging molecule for the treatment of a PSMA-expressing cancer, such as prostate cancer, may be from about 90 μg to about 1800 μg. In other embodiments, suitable therapeutic doses of a BCMA×CD3 bispecific T-cell engaging molecule for the treatment of a BCMA-positive cancer, such as multiple myeloma, may be from about 12,000 μg to about 19,500 μg. In certain embodiments, the priming dose may be lower than the therapeutic dose, e.g. a fraction of the therapeutic dose, such as about 10% to about 80% or about 15% to about 50% of the therapeutic dose. In alternative embodiments, the priming dose may be the same as the therapeutic dose. In embodiments in which a boost dose is administered, the boost dose may be a fraction of the priming dose, such as from about 10% to about 60% or from about 30% to about 40% of the priming dose.

In some embodiments, the methods of the invention further comprise administering a maintenance cycle of the bispecific T-cell engaging molecule to the patient after administration of the initiation cycle. The maintenance cycle may comprise administering the therapeutic dose of the bispecific T-cell engaging molecule by a bolus IV infusion or by subcutaneous injection at a dosing interval of at least 7 days. For example, in certain embodiments, the maintenance cycle comprises administering the therapeutic dose of the bispecific T-cell engaging molecule by a bolus IV infusion once every 7 days (e.g. weekly). In certain other embodiments, the maintenance cycle comprises administering the therapeutic dose of the bispecific T-cell engaging molecule by a bolus IV infusion once every 14 days (e.g. biweekly). In some embodiments, the therapeutic dose of the bispecific T-cell engaging molecule administered during the maintenance cycle is the same at each dosing interval (e.g. a fixed dose for the entire maintenance cycle). In these and other embodiments, the therapeutic dose and dosing frequency (e.g. weekly or biweekly) of the bispecific T-cell engaging molecule administered during the maintenance cycle is the same from one maintenance cycle to the next maintenance cycle. In any of the above-described embodiments, the duration of the maintenance cycle may be about 28 days.

In one embodiment in which the methods further comprise administering a maintenance cycle, the maintenance cycle is administered the following day after completing the initiation cycle, for example with no treatment-free periods between the initiation cycle and the maintenance cycle. In another embodiment, the maintenance cycle is administered about 7 days following the completion of the initiation cycle—i.e. there is a 7-day treatment-free period between the initiation cycle and the maintenance cycle. A patient may receive multiple maintenance cycles, such as 2, 3, 4, 5, 6, 7, 8, 9, 10, 11, or 12 or more maintenance cycles. In some embodiments, maintenance cycles are administered to the patient until the patient responds to treatment, for example achieves a complete response.

The bispecific T-cell engaging molecules employed in the methods of the invention generally comprise a first domain that specifically binds to a target cancer cell antigen (e.g. CEA, CD19, CD33, CD70, EGFRvIII, FLT3, GPRC5D, DLL3, BCMA, PSMA, STEAP1, STEAP2, MUC16, MUC17, or CLDN18.2), a second domain that specifically binds to human CD3, and a half-life extension domain that provides a half-life for the molecule of greater than 24 hours. The half-life extension domain can be an immunoglobulin Fc domain, a domain derived from serum albumin (e.g. human serum albumin), an albumin-binding domain (e.g. comprising human albumin binding peptides or an antibody fragment that specifically binds to serum albumin), peptides that bind to the neonatal Fc receptor (FcRn), and polyethylene glycol polymers. In certain embodiments, the bispecific T-cell engaging molecules used in the methods of the invention comprise an immunoglobulin Fc domain. In some such embodiments, the bispecific T-cell engaging molecule can be a bispecific antibody and have the general structure of a full-length immunoglobulin. For instance, in some embodiments, the bispecific T-cell engaging molecule can be a heterodimeric antibody comprising a light chain and heavy chain from an antibody that specifically binds to a target cancer cell antigen, and a light chain and heavy chain from an antibody that specifically binds to human CD3. In other embodiments, the bispecific T-cell engaging molecule employed in the methods of the invention comprises, in an amino to carboxyl order: (i) a first domain that specifically binds to a target cancer cell antigen; (ii) a second domain that specifically binds to human CD3; and (iii) an Fc domain comprising two Fc monomers, each monomer comprising an immunoglobulin hinge region, a CH2 domain, and a CH3 domain, wherein said two monomers are fused to each other via a peptide linker. In such embodiments, the bispecific T-cell engaging molecule can be a single chain polypeptide where all three domains are linked together, optionally via peptide linkers, to form a single polypeptide chain.

The patient to be treated according to the methods of the invention has or is diagnosed with cancer. In some embodiments, the cancer is a hematologic cancer, such as leukemia (e.g. acute myeloid leukemia, acute lymphocytic leukemia, chronic lymphocytic leukemia, chronic myeloid leukemia), myeloma (e.g. multiple myeloma), and lymphoma (e.g. diffuse large B-cell lymphoma, Burkitt lymphoma, and non-Hodgkin lymphoma). In other embodiments, the cancer may be a cancer selected from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma. In certain embodiments, the patient to be treated according to the methods of the invention has or is diagnosed with prostate cancer (e.g. metastatic castration-resistant prostate cancer) and the bispecific T-cell engaging molecule administered to the patient is a PSMA×CD3 bispecific T-cell engaging molecule. In one such embodiment, the PSMA×CD3 bispecific T-cell engaging molecule is a single chain polypeptide comprising the sequence of SEQ ID NO: 60. In certain other embodiments, the patient to be treated according to the methods of the invention has or is diagnosed with multiple myeloma (e.g. refractory and/or relapsed multiple myeloma) and the bispecific T-cell engaging molecule administered to the patient is a BCMA×CD3 bispecific T-cell engaging molecule. In one such embodiment, the BCMA×CD3 bispecific T-cell engaging molecule is a single chain polypeptide comprising the sequence of SEQ ID NO: 50.

The present invention also provides pharmaceutical compositions of bispecific T-cell engaging molecules for use in the methods described herein. The pharmaceutical compositions can comprise one or more pharmaceutically acceptable diluents, carriers, or excipients, including buffers, surfactants, and stabilizing agents. In certain embodiments, the pharmaceutical compositions comprise a bispecific T-cell engaging molecule, a buffer, a surfactant, and a stabilizing agent. In one embodiment, the pharmaceutical composition comprises a bispecific T-cell engaging molecule, a glutamate buffer, polysorbate 20 or polysorbate 80, and sucrose, at a pH of about 4.0 to about 4.4. In some embodiments, the pharmaceutical compositions may be lyophilized and reconstituted prior to administration to a patient.

In some embodiments, the present invention also provides kits comprising a pharmaceutical composition disclosed herein and instructions for using the pharmaceutical composition to prepare and deliver by intravenous infusion, priming doses, boost doses, and therapeutic doses of the bispecific T-cell engaging molecule for treating cancer in a patient in need thereof. In embodiments in which the pharmaceutical composition is provided in a lyophilized or dry powder form, the kit may comprise a diluent and instructions for reconstituting the pharmaceutical composition prior to administration. In certain embodiments, the kits may further comprise one or more vials of intravenous solution stabilizer (IVSS) and instructions for using the IVSS for pre-treatment of IV bags prior to dilution of the pharmaceutical composition for delivery to the patient.

The use of bispecific T-cell engaging molecules in any of the methods disclosed herein or for preparation of medicaments for administration according to any of the methods disclosed herein is specifically contemplated. For instance, the present invention includes a bispecific T-cell engaging molecule that specifically binds to a target cancer cell antigen and human CD3 for use in a method for treating cancer in a patient in need thereof, wherein the method comprises administering to the patient an initiation cycle of the bispecific T-cell engaging molecule, said initiation cycle comprising: administering a priming dose of the bispecific T-cell engaging molecule by continuous intravenous infusion over an extended period of time (e.g. 1 day to 7 days); and administering after the priming dose a therapeutic dose of the bispecific T-cell engaging molecule by a bolus intravenous infusion. In certain embodiments, the bispecific T-cell engaging molecule for use in the methods comprises a first domain that specifically binds to a target cancer cell antigen, a second domain that specifically binds to human CD3, and an Fc domain.

The present invention also includes the use of a bispecific T-cell engaging molecule that specifically binds to a target cancer cell antigen and human CD3 for the manufacture of a medicament for the treatment of cancer in a patient in need thereof, wherein the treatment comprises administering to the patient an initiation cycle of the bispecific T-cell engaging molecule, said initiation cycle comprising: administering a priming dose of the bispecific T-cell engaging molecule by continuous intravenous infusion over an extended period of time (e.g. 1 day to 7 days); and administering after the priming dose a therapeutic dose of the bispecific T-cell engaging molecule by a bolus intravenous infusion. In some such embodiments, the bispecific T-cell engaging molecule comprises a first domain that specifically binds to a target cancer cell antigen, a second domain that specifically binds to human CD3, and an Fc domain.

### BRIEF DESCRIPTION OF THE DRAWINGS

FIG. 1A shows the preliminary observed mean serum AMG 160 concentration-time profiles following administration of a 0.03 mg first dose administered as a 1-hour IV infusion (inverted triangles) or administered by continuous IV infusion over 72 hours (circles) during cycle 1. A 0.09 mg dose was administered 7 days after the first dose as a 1-hour IV infusion in both groups. Data are presented as mean±standard deviation.

FIG. 1B is an expanded view of FIG. 1A showing the preliminary AMG 160 concentration-time profiles over the first 7 days following administration of a 0.03 mg first dose administered as a 1-hour IV infusion (inverted triangles) or administered by continuous IV infusion over 72 hours (circles). The peak serum concentration (Cₘₐₓ) for AMG 160 is reduced by about 40% and occurs later when the first dose is administered by a continuous IV infusion as compared to administration of the same dose as a 1-hour IV infusion. Data are presented as mean±standard deviation.

FIG. 2 shows the preliminary observed mean serum AMG 160 concentration-time profiles following administration of a 0.09 mg dose administered as a 1-hour IV infusion (diamonds) or administered by continuous IV infusion over 72 hours (circles) during cycle 1. A 0.30 mg target dose was first administered 7 days after the 0.09 mg dose as a 1-hour IV infusion and then at biweekly intervals thereafter in both groups. Data are presented as mean±standard deviation.

FIG. 3A depicts serum interleukin-6 (IL-6) levels at different time points during the first 21 days of cycle 1 (C1) for patients dosed with AMG 160 in cIV cohort 1 (cohort 1_eIV). Patients in cIV cohort 1 received a 0.03 mg priming dose of AMG 160 administered over the first 3 days of cycle 1 at a constant rate (e.g. 0.01 mg/day for 3 days) and received a 0.09 mg target dose of AMG 160 administered by a 1-hour IV infusion on day 8 of cycle 1 (C1D8). Each line and symbol type represent data from an individual patient. Arrows at the top of the figure indicate timing of AMG 160 dose administrations. The dotted lines at the top and bottom of the figure represent upper limit of quantitation (ULOQ) and lower limit of quantitation (LLOQ) for IL-6, respectively.

FIG. 3B depicts serum IL-6 levels at different time points during the first 21 days of cycle 1 (C1) for patients dosed with AMG 160 in cIV cohorts 2a and 2b (cohort 2_eIV). Patients in cIV cohort 2a and 2b received a 0.09 mg priming dose of AMG 160 administered over the first 2 days (cohort 2b) or first 3 days (cohort 2a) of cycle 1 at a constant rate and received a 0.30 mg target dose of AMG 160 administered by a 1-hour IV infusion on day 8 of cycle 1 (C1D8). Each line and symbol type represent data from an individual patient. Arrows at the top of the figure indicate timing of AMG 160 dose administrations. The dotted lines at the top and bottom of the figure represent ULOQ and LLOQ for IL-6, respectively.

FIG. 3C depicts serum IL-6 levels at different time points during the first 21 days of cycle 1 (C1) for patients dosed with AMG 160 in cohort 6b. Patients in cohort 6b received a first priming dose of 0.03 mg of AMG 160 on day 1 (D1), a second priming dose of 0.09 mg of AMG 160 on day 8 (D8), and a target dose of 0.90 mg of AMG 160 on day 15 (D15), where all AMG 160 doses were administered as 1-hour IV infusions. Each line and symbol type represent data from an individual patient. Arrows at the top of the figure indicate timing of AMG 160 dose administrations. The dotted lines at the top and bottom of the figure represent ULOQ and LLOQ for IL-6, respectively.

FIG. 3D depicts serum IL-6 levels at different time points during the first 21 days of cycle 1 (C1) for patients dosed with AMG 160 in cohort 5. Patients in cohort 5 received a first priming dose of 0.01 mg of AMG 160 on day 1 (D1), a second priming dose of 0.09 mg of AMG 160 on day 8 (D8), and a target dose of 0.30 mg of AMG 160 on day 15 (D15), where all AMG 160 doses were administered as 1-hour IV infusions. Each line and symbol type represent data from an individual patient. Arrows at the top of the figure indicate timing of AMG 160 dose administrations. The dotted lines at the top and bottom of the figure represent ULOQ and LLOQ for IL-6, respectively.

FIG. 4A shows serum tumor necrosis factor-alpha (TNF-alpha) levels at different time points during the first 21 days of cycle 1 (C1) for patients dosed with AMG 160 in cIV cohort 1 (cohort 1_eIV). Patients in cIV cohort 1 received a 0.03 mg priming dose of AMG 160 administered over the first 3 days of cycle 1 at a constant rate (e.g. 0.01 mg/day for 3 days) and received a 0.09 mg target dose of AMG 160 administered by a 1-hour IV infusion on day 8 of cycle 1 (C1D8). Each line and symbol type represent data from an individual patient. Arrows at the top of the figure indicate timing of AMG 160 dose administrations.

FIG. 4B shows serum TNF-alpha levels at different time points during the first 21 days of cycle 1 (C1) for patients dosed with AMG 160 in cIV cohorts 2a and 2b (cohort 2_eIV). Patients in cIV cohort 2a and 2b received a 0.09 mg priming dose of AMG 160 administered over the first 2 days (cohort 2b) or first 3 days (cohort 2a) of cycle 1 at a constant rate and received a 0.30 mg target dose of AMG 160 administered by a 1-hour IV infusion on day 8 of cycle 1 (C1D8). Each line and symbol type represent data from an individual patient. Arrows at the top of the figure indicate timing of AMG 160 dose administrations.

FIG. 4C shows serum TNF-alpha levels at different time points during the first 21 days of cycle 1 (C1) for patients dosed with AMG 160 in cohort 6b. Patients in cohort 6b received a first priming dose of 0.03 mg of AMG 160 on day 1 (D1), a second priming dose of 0.09 mg of AMG 160 on day 8 (D8), and a target dose of 0.90 mg of AMG 160 on day 15 (D15), where all AMG 160 doses were administered as 1-hour IV infusions. Each line and symbol type represent data from an individual patient. Arrows at the top of the figure indicate timing of AMG 160 dose administrations.

FIG. 4D shows serum TNF-alpha levels at different time points during the first 21 days of cycle 1 (C1) for patients dosed with AMG 160 in cohort 5. Patients in cohort 5 received a first priming dose of 0.01 mg of AMG 160 on day 1 (D1), a second priming dose of 0.09 mg of AMG 160 on day 8 (D8), and a target dose of 0.30 mg of AMG 160 on day 15 (D15), where all AMG 160 doses were administered as 1-hour IV infusions. Each line and symbol type represent data from an individual patient. Arrows at the top of the figure indicate timing of AMG 160 dose administrations.

FIG. 5A depicts serum interferon-gamma (IFN-gamma) levels at different time points during the first 21 days of cycle 1 (C1) for patients dosed with AMG 160 in cIV cohort 1 (cohort 1_eIV). Patients in cIV cohort 1 received a 0.03 mg priming dose of AMG 160 administered over the first 3 days of cycle 1 at a constant rate (e.g. 0.01 mg/day for 3 days) and received a 0.09 mg target dose of AMG 160 administered by a 1-hour IV infusion on day 8 of cycle 1 (C1D8). Each line and symbol type represent data from an individual patient. Arrows at the top of the figure indicate timing of AMG 160 dose administrations. The dotted lines at the top and bottom of the figure represent ULOQ and LLOQ for IFN-gamma, respectively.

FIG. 5B depicts serum IFN-gamma levels at different time points during the first 21 days of cycle 1 (C1) for patients dosed with AMG 160 in cIV cohorts 2a and 2b (cohort 2_eIV). Patients in cIV cohort 2a and 2b received a 0.09 mg priming dose of AMG 160 administered over the first 2 days (cohort 2b) or first 3 days (cohort 2a) of cycle 1 at a constant rate and received a 0.30 mg target dose of AMG 160 administered by a 1-hour IV infusion on day 8 of cycle 1 (C1D8). Each line and symbol type represent data from an individual patient. Arrows at the top of the figure indicate timing of AMG 160 dose administrations. The dotted lines at the top and bottom of the figure represent ULOQ and LLOQ for IFN-gamma, respectively.

FIG. 5C depicts serum IFN-gamma levels at different time points during the first 21 days of cycle 1 (C1) for patients dosed with AMG 160 in cohort 6b. Patients in cohort 6b received a first priming dose of 0.03 mg of AMG 160 on day 1 (D1), a second priming dose of 0.09 mg of AMG 160 on day 8 (D8), and a target dose of 0.90 mg of AMG 160 on day 15 (D15), where all AMG 160 doses were administered as 1-hour IV infusions. Each line and symbol type represent data from an individual patient. Arrows at the top of the figure indicate timing of AMG 160 dose administrations. The dotted lines at the top and bottom of the figure represent ULOQ and LLOQ for IFN-gamma, respectively.

FIG. 5D depicts serum IFN-gamma levels at different time points during the first 21 days of cycle 1 (C1) for patients dosed with AMG 160 in cohort 5. Patients in cohort 5 received a first priming dose of 0.01 mg of AMG 160 on day 1 (D1), a second priming dose of 0.09 mg of AMG 160 on day 8 (D8), and a target dose of 0.30 mg of AMG 160 on day 15 (D15), where all AMG 160 doses were administered as 1-hour IV infusions. Each line and symbol type represent data from an individual patient. Arrows at the top of the figure indicate timing of AMG 160 dose administrations. The dotted lines at the top and bottom of the figure represent ULOQ and LLOQ for IFN-gamma, respectively.

FIG. 6A shows C-reactive protein (CRP) levels in cynomolgus monkeys administered intravenous injections of a CDH3×MSLN T-cell engaging molecule at a dose of either 1000 μg/kg (animals 2805 and 2807) or 5000 μg/kg (animal 2808) on each of study days 1, 2, 3, 4, 5, 6, 7, 8, and 15.

FIG. 6B shows CRP levels in cynomolgus monkeys administered a CDH3×MSLN T-cell engaging molecule according to a dosing regimen of either (i) a dose of 7000 μg/kg by continuous IV infusion over 7 days (e.g. 1000 μg/kg/day) followed by 1000 μg/kg intravenous injections on study days 8 and 15 (animals 2810 and 2811) or (ii) a dose of 35000 μg/kg by continuous IV infusion over 7 days (e.g. 5000 μg/kg/day) followed by 5000 ag/kg intravenous injections on study days 8 and 15 (animal 2812).

FIG. 7A shows CD25+ T cell activation in cynomolgus monkeys administered intravenous injections of a CDH3×MSLN T-cell engaging molecule at a dose of either 1000 g/kg (animals 2805 and 2807) or 5000 μg/kg (animal 2808) on each of study days 1, 2, 3, 4, 5, 6, 7, 8, and 15.

FIG. 7B shows CD25+ T cell activation in cynomolgus monkeys administered a CDH3×MSLN T-cell engaging molecule according to a dosing regimen of either (i) a dose of 7000 g/kg by continuous IV infusion over 7 days (e.g. 1000 μg/kg/day) followed by 1000 μg/kg intravenous injections on study days 8 and 15 (animals 2810 and 2811) or (ii) a dose of 35000 μg/kg by continuous IV infusion over 7 days (e.g. 5000 μg/kg/day) followed by 5000 ag/kg intravenous injections on study days 8 and 15 (animal 2812).

FIG. 8A shows CD69+ T cell activation in cynomolgus monkeys administered intravenous injections of a CDH3×MSLN T-cell engaging molecule at a dose of either 1000 μg/kg (animals 2805 and 2807) or 5000 μg/kg (animal 2808) on each of study days 1, 2, 3, 4, 5, 6, 7, 8, and 15.

FIG. 8B shows CD69+ T cell activation in cynomolgus monkeys administered a CDH3×MSLN T-cell engaging molecule according to a dosing regimen of either (i) a dose of 7000 μg/kg by continuous IV infusion over 7 days (e.g. 1000 μg/kg/day) followed by 1000 μg/kg intravenous injections on study days 8 and 15 (animals 2810 and 2811) or (ii) a dose of 35000 μg/kg by continuous IV infusion over 7 days (e.g. 5000 μg/kg/day) followed by 5000 μg/kg intravenous injections on study days 8 and 15 (animal 2812).

### DETAILED DESCRIPTION

Bispecific T-cell engaging molecules are a new class of immunotherapies that are being developed for the treatment of various cancers. These molecules are designed to direct a patient's T cells to cancer cells to induce the T-cells to attack and kill the cancer cells. Newer bispecific T-cell engaging molecules have been designed to comprise half-life extension moieties to offer more convenient, less frequent administrations than the first-generation bispecific T-cell engaging molecules that are necessarily administered by a continuous infusion over the course of weeks owing to their short half-lives of less than one day. As a result of the mechanism of action of bispecific T-cell engaging molecules, CRS is a possible adverse event that can occur in patients when first administered with a bispecific T-cell engaging molecule. CRS events can prevent, limit, or delay the administration of doses to the patient necessary to achieve the desired therapeutic efficacy. In the case of the half-life extended (HLE) bispecific T-cell engaging molecules which are typically administered as a bolus injection or infusion at weekly dosing intervals or longer dosing intervals, the ability to adapt a dosing regimen to reduce or avoid CRS events in a patient is particularly challenging. It has been observed that peak serum drug levels (Cₘₐₓ) following a bolus infusion of the first dose of an HLE bispecific T-cell engaging molecule in cycle 1 correlate with the degree of CRS events in patients (see Example 1). One possible approach to minimize a rapid increase in drug exposure following administration of an initial dose is to employ a step-dosing strategy whereby a lower dose of the bispecific T-cell engaging molecule is initially administered followed by administration of one or more dose steps up to a therapeutic dose. However, such an approach may require that the therapeutic dose of the bispecific T-cell engaging molecule is not administered until several weeks following initiation of treatment and achievement of therapeutic doses may not be possible even with multiple steps.

The present invention addresses these challenges by providing administration regimens for bispecific T-cell engaging molecules, particularly HLE bispecific T-cell engaging molecules, that deliver therapeutic doses as early as possible in the first cycle of treatment to maximize efficacy while minimizing the occurrence and/or severity of CRS and other adverse events. Accordingly, in one aspect, the present invention provides a method for administering a therapeutic dose of a bispecific T-cell engaging molecule to a patient diagnosed with cancer comprising administering to the patient an initiation cycle comprising: (i) administering a priming dose of the bispecific T-cell engaging molecule by continuous intravenous infusion over a period of time (e.g. 1 day to 7 days); and (ii) administering after the priming dose a therapeutic dose of the bispecific T-cell engaging molecule by a bolus intravenous infusion or subcutaneous injection. Without being bound by theory, it is believed that administration of the first dose (i.e. priming dose) of the bispecific T-cell engaging molecule by a continuous IV infusion over an extended period of time will avoid sharp increases to peak serum concentrations (Cₘₐₓ) of the molecule and decrease and delay Cₘₐₓ, thereby reducing the frequency and severity of CRS and other adverse events, as well as maintain high levels of cumulative drug exposures during the dosing interval to enable achievement of efficacious doses as early as possible in the initiation cycle, thereby translating into enhanced efficacy in eliminating cancer cells. Thus, administration of the bispecific T-cell engaging molecules according to the methods of the invention improves the safety profile of the molecules by reducing adverse events, particularly CRS events, and enhances the efficacy of the molecules by achieving efficacious exposure levels during the first week of treatment. Early T-cell activation leads to a substantial release of cytokines by the T-cells, which causes a cascading amplification of cytokine release by other resident cells in the tumor microenvironment, such as macrophages and monocytes. After prolonged activation by a bispecific T-cell engager molecule, T-cells downregulate the production of cytokines, possibly by a feedback loop mechanism, but continue to be able to recognize and kill cancer cells. The downregulation of cytokine production in the T-cells induced by prolonged exposure to the bispecific T-cell engager molecule is referred to herein as “priming” of the T-cells. It is also believed that by administering a priming dose of the bispecific T-cell engaging molecule by a continuous IV infusion over an extended period according to the methods of the invention allows for the gradual priming of a patient's T-cells, such that administration of a higher therapeutic dose produces a reduced or minimal cytokine release and associated CRS events.

Generally, the methods of the invention comprise administering a bispecific T-cell engaging molecule to the patient in one or more treatment cycles. A “treatment cycle” or “cycle” refers to a period of administration of the bispecific T-cell engaging molecule at specific dosages and dosing intervals. According to the methods of the invention, a patient can receive multiple treatment cycles (e.g. 2, 3, 4, 5, 6, 7, 8, 9, 10, 11, 12, 13, 14 or more cycles). The treatment cycles can be administered to the patient consecutively with no break or period without administration of the bispecific T-cell engaging molecule between the cycles. Alternatively, a period without administration of the bispecific T-cell engaging molecule (e.g. a “treatment-free period” or “break”) can be employed between the treatment cycles. The length of the treatment-free period can be adjusted based on the patient's characteristics and/or response to treatment.

In some embodiments, the methods of the invention comprise administering a bispecific T-cell engaging molecule to the patient in at least one initiation cycle. As used herein, an “initiation cycle” is a treatment cycle in which the bispecific T-cell engaging molecule is administered at two or more different doses at a dosing frequency and mode of administration designed to minimize adverse events, for example, such as adverse events associated with CRS, while enabling exposure of the patient to a therapeutic dose of the bispecific T-cell engaging molecule in the shortest time possible. An initiation cycle is preferably administered to a patient as the first treatment cycle when the patient begins a course of treatment with the bispecific T-cell engaging molecule. An initiation cycle may also be administered to a patient when the patient re-starts a course of treatment with the bispecific T-cell engaging molecule, for example, following a treatment-free period, dosing interruption (e.g. when a patient didn't complete a previous treatment cycle), or a relapse or progression of a cancer in the patient. Although administration of one initiation cycle will typically be sufficient, in some embodiments of the methods of the invention, administration of two or more initiation cycles is contemplated. In one particular embodiment, only one initiation cycle is administered to the patient.

In certain embodiments, the initiation cycle comprises administering a priming dose of the bispecific T-cell engaging molecule by continuous intravenous infusion over an extended period of time. As used herein, the term “priming dose” refers to a dose or amount of a bispecific T-cell engaging molecule that primes a patient for subsequent administration of a therapeutic dose of the bispecific T-cell engaging molecule such that administration of the therapeutic dose produces fewer or less severe adverse events, e.g. fewer or less severe CRS events, in the patient.

In some embodiments, the priming dose may be lower than a therapeutic dose but is a dose that is sufficient to prime a patient's T-cells, e.g. to release cytokines, such that administration of a subsequent greater dose or therapeutic dose of the bispecific T-cell engaging molecule produces an attenuated increase in cytokine secretion. In certain embodiments, the priming dose is sufficient to increase the proportion of activated peripheral T-cells in the patient (e.g. increases the proportion of CD69+CD8+ peripheral T-cells) relative to the proportion of activated T-cells in the patient prior to receiving the dose of the bispecific T-cell engaging molecule. In some embodiments, the priming dose may be a fraction of the therapeutic dose. For example, in some embodiments, the priming dose may be from about 10% to about 80% of the therapeutic dose, such as from about 20% to about 75%, from about 15% to about 50%, from about 25% to about 60%, or from about 30% to about 50% of the therapeutic dose. In one embodiment, the priming dose is about 25% of the therapeutic dose. In another embodiment, the priming dose is about 30% of the therapeutic dose. In yet another embodiment, the priming dose is about 50% of the therapeutic dose.

In other embodiments, the priming dose may be the same as or even higher than the therapeutic dose, such as, for example, 1.5 times or twice the therapeutic dose. In some such embodiments, continuous intravenous infusion of the priming dose can be used to achieve therapeutic exposure levels within 24 hours to 96 hours following the start of continuous infusion of the priming dose without causing the same number or severity of adverse events as administration of the same dose administered by a bolus intravenous infusion. In some embodiments, the priming dose of the bispecific T-cell engaging molecule is a dose that provides a steady state concentration (Cₛₛ) in the blood of the bispecific T-cell engaging molecule above the EC50 (i.e. half maximal effective concentration) determined in a T-cell cytotoxicity assay or animal tumor model (e.g. xenograft mouse model) appropriate for evaluating the potency of the bispecific T-cell engaging molecule. In other embodiments, the priming dose of the bispecific T-cell engaging molecule is a dose that provides a Cₛₛ in the blood of the bispecific T-cell engaging molecule above the EC90 (i.e. 90% maximal effective concentration) determined in a T-cell cytotoxicity assay or an animal tumor model (e.g. xenograft mouse model) appropriate for evaluating the potency of the bispecific T-cell engaging molecule. The specific amounts of the priming dose may vary depending on the specific bispecific T-cell engaging molecule employed in the method, the type, grade or stage of cancer to be treated in the patient, and one or more patient characteristics, such as age, co-morbidities, and other concomitant medications. Suitable priming doses for any particular bispecific T-cell engaging molecule can be determined according to the guidance provided herein from a given therapeutic dose of the bispecific T-cell engaging molecule, such as those described in further detail below, to be administered to the patient for the treatment of a specific type of cancer.

The term “therapeutic dose” refers to a dose or amount of a bispecific T-cell engaging molecule sufficient to treat or ameliorate a cancer or one or more of its symptoms, particularly a state or symptoms associated with the cancer, or otherwise prevent, hinder, retard or reverse the progression of the cancer or any other undesirable symptom associated with the cancer in any way whatsoever. The amounts of the therapeutic dose may vary depending on the characteristics of the patient to be treated, the type, grade or stage of cancer diagnosed in the patient, and the specific bispecific T-cell engaging molecule administered to the patient. Specific therapeutic doses for bispecific T-cell engaging molecules can be determined from dose-exploration human clinical trials, such as those described in the Examples, or may in some cases be estimated from relevant animal models for the particular cancer to be treated. Exemplary ranges of therapeutic doses of a bispecific T-cell engaging molecule for the treatment of cancer may include, but are not limited to, doses of about 50 μg to about 200 mg, from about 200 μg to about 80 mg, from about 90 μg to about 30 mg, from about 300 μg to about 15 mg, from about 150 μg to about 2 mg, from about 6 mg to about 25 mg, from about 1 mg to about 20 mg, from about 10 mg to about 100 mg, or from about 50 mg to about 150 mg.

In preferred embodiments of the methods of the invention, the priming dose of the bispecific T-cell engaging molecule is administered to the patient by a continuous intravenous infusion over an extended period of time. As used herein, a continuous intravenous infusion refers to a controlled method of intravenous administration of the bispecific T-cell engaging molecule given over a period of time longer than about 3 hours, more typically longer than about 6 hours, without interruption or without substantial interruption. The continuous intravenous infusion may be administered by way of a fluid delivery device or small pump system including a fluid driving mechanism for driving fluid out of a reservoir and an actuating mechanism for actuating the driving mechanism. Pump systems for such administration may include a needle or a cannula for penetrating the skin of a patient and delivering the infusion solution into the patient's body. The pump system can be connected to the patient for 24 hours up to several days. Pump systems for delivering intravenous infusions are known in the art. Depending on the duration of the continuous infusion, the bags or reservoirs containing the infusion solution in the pump system may need to be exchanged or replaced. During the exchange of the bag or reservoir in the pump system, a temporary interruption of the otherwise uninterrupted flow of the infusate may occur. Such temporary interruptions occurring from bag or reservoir replacement do not constitute an interruption or substantial interruption of the intravenous administration and the period of time during which the bag or reservoir is replaced would still be considered to be within the period of a continuous intravenous infusion as the term is used herein.

In some embodiments of the methods of the invention, the priming dose of the bispecific T-cell engaging molecule is administered to the patient by a continuous intravenous infusion over a period of at least 24 hours, for example over a period of 1 to 14 days, 1 to 7 days, or 1 to 5 days. In one embodiment, the priming dose of the bispecific T-cell engaging molecule is administered to the patient by a continuous intravenous infusion over a period of about 7 days. In another embodiment, the priming dose of the bispecific T-cell engaging molecule is administered to the patient by a continuous intravenous infusion over a period of about 5 days. In another embodiment, the priming dose of the bispecific T-cell engaging molecule is administered to the patient by a continuous intravenous infusion over a period of about 4 days. In yet another embodiment, the priming dose of the bispecific T-cell engaging molecule is administered to the patient by a continuous intravenous infusion over a period of about 3 days. In still another embodiment, the priming dose of the bispecific T-cell engaging molecule is administered to the patient by a continuous intravenous infusion over a period of about 2 days. In these and other embodiments, the continuous intravenous infusion is given at a constant flow rate—that is the continuous intravenous infusion delivers the priming dose at a constant rate over the period of the infusion. By way of example, for a priming dose of 8.4 mg, a continuous intravenous infusion at a constant flow rate given over 7 days would deliver the priming dose at a rate of 1.2 mg per day such that the total priming dose of 8.4 mg would be delivered at the completion of the 7-day infusion period. Alternatively, in some embodiments, the continuous intravenous infusion may be given at a variable flow rate such that the priming dose is delivered at different doses per day over the period of infusion. For instance, in one such embodiment, the flow rate of the continuous infusion can be adjusted such that increasing doses are given each day over the infusion period to deliver the total priming dose at the completion of the infusion period.

The duration of the continuous intravenous infusion period can be selected to reduce the peak concentration (Cₘₐₓ) resulting from a given dose of the bispecific T-cell engaging molecule in the blood by at least about 20% as compared to the Cₘₐₓ achieved with the same dose administered by a bolus intravenous infusion. For example, the priming dose of the bispecific T-cell engaging molecule is administered by continuous intravenous infusion over a period sufficient to reduce Cₘₐₓ of the bispecific T-cell engaging molecule by at least about 30%, at least about 40%, at least about 50%, at least about 60%, or at least about 70% relative to the Cₘₐₓ achieved when the priming dose is administered by a bolus intravenous infusion. In such embodiments, the time to Cₘₐₓ is delayed to the end of the infusion period. For instance, in some embodiments of the methods of the invention, the priming dose of the bispecific T-cell engaging molecule is administered by continuous intravenous infusion such that a Cₘₐₓ of the bispecific T-cell engaging molecule is achieved later than 24 hours following the start of the infusion, e.g. in 2 days, in 3 days, in 4 days, in 5 days, in 6 days, in 7 days, or later following the start of the continuous intravenous infusion.

In certain embodiments of the methods of the invention, the priming dose and duration of continuous intravenous infusion is selected to provide a steady state concentration (Cₛₛ) in the blood of the bispecific T-cell engaging molecule within 1 to 7 days, for example, within 1 day, 2 days, 3 days, 4 days, 5 days, 6 days, or 7 days following the start of the continuous intravenous infusion. In one embodiment, the priming dose of the bispecific T-cell engaging molecule is administered by continuous intravenous infusion such that a Cₛₛ of the bispecific T-cell engaging molecule is achieved within 2 to 4 days following the start of the continuous intravenous infusion. In another embodiment, the priming dose of the bispecific T-cell engaging molecule is administered by continuous intravenous infusion such that a Cₛₛ of the bispecific T-cell engaging molecule is achieved within 1 to 2 days following the start of the continuous intravenous infusion. In yet another embodiment, the priming dose of the bispecific T-cell engaging molecule is administered by continuous intravenous infusion such that a Cₛₛ of the bispecific T-cell engaging molecule is achieved within 3 to 5 days following the start of the continuous intravenous infusion. In these and other embodiments, the Cₛₛ of the bispecific T-cell engaging molecule is a therapeutic exposure level, e.g. above the EC50 or EC90 of the molecule in an appropriate T-cell cytotoxicity assay, an animal tumor model, or other preclinical model.

In some embodiments of the methods of the invention, the initiation cycle comprises administering a therapeutic dose of the bispecific T-cell engaging molecule by a bolus intravenous infusion following administration of the priming dose. As used herein, a bolus intravenous infusion, used interchangeably herein with short-term intravenous infusion, refers to an intravenous infusion of a small volume (e.g. 20 mL to 100 mL) administered over a period of, at most three hours, and more typically over a period of about 30 min to about 90 min. In some embodiments of the methods of the invention, a bolus intravenous infusion is an intravenous infusion administered over about 30 min to about 60 min. In certain embodiments of the methods of the invention, a bolus intravenous infusion is an intravenous infusion administered over about 60 min (e.g. 55 min to 65 min). In other embodiments of the methods of the invention, the initiation cycle comprises administering a therapeutic dose of the bispecific T-cell engaging molecule by a subcutaneous injection following administration of the priming dose.

Following administration of the priming dose by continuous intravenous infusion, therapeutic doses of the bispecific T-cell engaging molecule can be administered by a bolus intravenous infusion or a subcutaneous injection at a dosing interval of at least 7 days for the duration of the initiation cycle. For example, in some embodiments, the therapeutic dose of the bispecific T-cell engaging molecule is administered once every 7 days (e.g. QW or weekly dosing) for the duration of the initiation cycle. In other embodiments, the therapeutic dose of the bispecific T-cell engaging molecule is administered once every 14 days (e.g. Q2W or biweekly dosing) for the duration of the initiation cycle. Depending on the half-life of the bispecific T-cell engaging molecule and the duration of the initiation cycle, the therapeutic dose of the bispecific T-cell engaging molecule may be administered at longer dosing intervals, such as once every three weeks or once every four weeks for the remainder of the initiation cycle.

During the initiation cycle, the therapeutic dose of the bispecific T-cell engaging molecule can be administered immediately following (e.g. on the same day or the next day) the completion of the continuous intravenous infusion period of the priming dose. Alternatively, the therapeutic dose of the bispecific T-cell engaging molecule may be administered after a delay of one or more days following the completion of the continuous intravenous infusion period of the priming dose. In certain embodiments, the period between the completion of the continuous intravenous infusion of the priming dose and administration (e.g. by bolus intravenous infusion or subcutaneous injection) of the therapeutic dose is adjusted to maintain serum exposures of the bispecific T-cell engaging molecule at or substantially at the exposure level attained at the end of the continuous intravenous infusion period. In certain embodiments of the methods of the invention, the therapeutic dose is administered by a bolus intravenous infusion on the same day the continuous intravenous infusion of the priming dose ends. For example, in such embodiments, the therapeutic dose may be administered within 18 hours, 16 hours, 12 hours, 8 hours, 6 hours, 4 hours, 3 hours, 2 hours, 1 hour, or 30 minutes of the completion of the continuous intravenous infusion of the priming dose. In some embodiments of the methods of the invention, the therapeutic dose is administered by a bolus intravenous infusion about 1 day to about 7 days after completion of the continuous intravenous infusion of the priming dose during the initiation cycle. For instance, in one embodiment, the therapeutic dose is administered about 1 day (e.g. the next day) after administration of the priming dose. In another embodiment, the therapeutic dose is administered about 3 days after administration of the priming dose. In another embodiment, the therapeutic dose is administered about 4 days after administration of the priming dose. In yet another embodiment, the therapeutic dose is administered about 5 days after administration of the priming dose. In still another embodiment, the therapeutic dose is administered about 6 days after administration of the priming dose.

In certain embodiments of the methods of the invention, the initiation cycle further comprises administering a boost dose of the bispecific T-cell engaging molecule by a bolus intravenous infusion or subcutaneous injection after the priming dose and before the therapeutic dose. A “boost dose” of the bispecific T-cell engaging molecule may be used to maintain exposure levels (e.g. Cₛₛ) of the bispecific T-cell engaging molecule achieved with the continuous intravenous infusion of the priming dose between the period after completion of the continuous infusion period and prior to administration of the therapeutic dose. The boost dose will generally be a fraction of the priming dose, such as about 10% to about 60% of the priming dose, for example, about 10%, about 15%, about 20%, about 25%, about 30%, about 35%, about 40%, about 45%, about 50%, about 55%, or about 60% of the priming dose. In some embodiments, the boost dose is about 30% to about 40% of the priming dose. In other embodiments, the boost dose is about 25% to about 50% of the priming dose. Implementation of a boost dose is particularly useful in embodiments in which there is a delay of two or more days between completion of the continuous infusion of the priming dose and administration of the therapeutic dose. In some embodiments of the methods of the invention, the boost dose of the bispecific T-cell engaging molecule is administered on the same day the continuous intravenous infusion of the priming dose ends. For example, in such embodiments, the boost dose may be administered within 18 hours, 16 hours, 12 hours, 8 hours, 6 hours, 4 hours, 3 hours, 2 hours, 1 hour, or 30 minutes of the completion of the continuous intravenous infusion of the priming dose. In certain embodiments, a boost dose of the bispecific T-cell engaging molecule is administered 1 day (e.g. next day) following completion of the continuous intravenous infusion of the priming dose and at least 2 days, 3 days, 4 days, 5 days, or 6 days before the administration of the therapeutic dose. In other embodiments, a boost dose of the bispecific T-cell engaging molecule is administered 2 days following completion of the continuous intravenous infusion of the priming dose and at least 2 days, 3 days, 4 days, or 5 days before the administration of the therapeutic dose.

In certain embodiments of the methods of the invention, the duration of the initiation cycle is from about 14 days to about 56 days, for example, from about 14 days to about 28 days, from about 21 days to about 42 days, from about 28 days to about 49 days, or from about 21 days to about 28 days. In certain embodiments, the duration of the initiation cycle is about 28 days. In such embodiments, a priming dose of the bispecific T-cell engaging molecule may be administered by continuous intravenous infusion over days 1 to 3 of the initiation cycle and a therapeutic dose of the bispecific T-cell engaging molecule may be administered by bolus intravenous infusion on days 8 and 22 of the initiation cycle. In other such embodiments, a priming dose of the bispecific T-cell engaging molecule may be administered by continuous intravenous infusion over days 1 to 2 of the initiation cycle and a therapeutic dose of the bispecific T-cell engaging molecule may be administered by bolus intravenous infusion on days 8 and 22 of the initiation cycle. In certain embodiments in which the duration of the initiation cycle is about 28 days, a priming dose of the bispecific T-cell engaging molecule is administered by continuous intravenous infusion over days 1 to 2 of the initiation cycle and a therapeutic dose of the bispecific T-cell engaging molecule is administered by bolus intravenous infusion on days 8, 15, and 22 of the initiation cycle. In related embodiments, a priming dose of the bispecific T-cell engaging molecule is administered by continuous intravenous infusion over days 1 to 2 of the initiation cycle, a boost dose of the bispecific T-cell engaging molecule is administered by bolus intravenous infusion on day 3 of the initiation cycle, and a therapeutic dose of the bispecific T-cell engaging molecule is administered by bolus intravenous infusion on days 8, 15, and 22 of the initiation cycle. In certain other embodiments in which the duration of the initiation cycle is about 28 days, a priming dose of the bispecific T-cell engaging molecule is administered by continuous intravenous infusion over days 1 to 7 of the initiation cycle and a therapeutic dose of the bispecific T-cell engaging molecule is administered by bolus intravenous infusion on days 8, 15, and 22 of the initiation cycle. In still other embodiments in which the duration of the initiation cycle is about 28 days, a priming dose of the bispecific T-cell engaging molecule is administered by continuous intravenous infusion over days 1 to 4 of the initiation cycle and a therapeutic dose of the bispecific T-cell engaging molecule is administered by bolus intravenous infusion on days 8, 15, and 22 of the initiation cycle.

In some embodiments, the methods of the invention further comprise administering to the patient at least one maintenance cycle of the bispecific T-cell engaging molecule after administration of one or more initiation cycles. As used herein, a “maintenance cycle” is a treatment cycle in which the bispecific T-cell engaging molecule is administered at a dosing frequency designed to maintain a threshold level of exposure of the bispecific T-cell engaging molecule at therapeutic levels in the patient. In some embodiments, the dosing frequency employed in the maintenance cycle is lower than the dosing frequency employed in the initiation cycle (i.e. the dosing interval in the maintenance cycle is longer than the dosing interval in the initiation cycle). In certain embodiments, the maintenance cycle is administered immediately after the completion of one or more initiation cycles. Accordingly, in such embodiments, there are no treatment-free periods or breaks between the end of the initiation cycle and the start of the maintenance cycle. In one such embodiment, the maintenance cycle is administered the following day after completing the initiation cycle. In other embodiments, there is a treatment-free period or break between the completion of the initiation cycle and the administration of the maintenance cycle. Preferably, the treatment-free period between the initiation cycle and the maintenance cycle is no longer than the dosing interval employed in the maintenance cycle. In one embodiment, the maintenance cycle is administered about 7 days following completion of the initiation cycle. In another embodiment, the maintenance cycle is administered about 14 days following completion of the initiation cycle.

Multiple maintenance cycles (e.g. 2, 3, 4, 5, 6, 7, 8, 9, 10, 11, 12 or more cycles) can be administered to the patient depending on the desired duration of treatment for that patient. For instance, the patient may receive maintenance cycles of the bispecific T-cell engaging molecule until the patient achieves a desired level of response, such as a complete response or partial response. In some embodiments, two or more maintenance cycles are administered to the patient. In other embodiments, four or more maintenance cycles are administered to the patient. In still other embodiments, six to twelve maintenance cycles are administered to the patient. In certain embodiments, the maintenance cycles are administered consecutively with no treatment-free periods between the maintenance cycles. If a treatment interruption is necessary, ideally the duration of the treatment-free period will be no greater than twice the dosing interval employed in the maintenance cycle. By way of example, if the dosing interval employed in the maintenance cycle is once every 14 days (e.g. biweekly), the treatment-free period between maintenance cycles will preferably be about 28 days or less.

In certain embodiments of the methods of the invention, the maintenance cycle comprises administering the bispecific T-cell engaging molecule at any of the therapeutic doses as described herein by a bolus intravenous infusion or subcutaneous injection at a dosing interval of at least 7 days. For instance, in some embodiments of the methods of the invention, the maintenance cycle comprises administering a therapeutic dose of the bispecific T-cell engaging molecule by a bolus intravenous infusion or subcutaneous injection once every 7 days (e.g. weekly, QW dosing). In other embodiments of the methods of the invention, the maintenance cycle comprises administering a therapeutic dose of the bispecific T-cell engaging molecule by a bolus intravenous infusion or subcutaneous injection once every 14 days (e.g. biweekly, Q2W dosing). In still other embodiments, the therapeutic dose of the bispecific T-cell engaging molecule may be administered by a bolus intravenous infusion or subcutaneous injection at longer dosing intervals during the maintenance cycle, such as once every three weeks or once every four weeks. Preferably, the therapeutic dose of the bispecific T-cell engaging molecule administered during the maintenance cycle is the same at each dosing interval, e.g., each weekly or biweekly dosing interval (e.g. a fixed dose for the entire maintenance cycle). In these and other embodiments, the therapeutic dose and dosing frequency of the bispecific T-cell engaging molecule administered during the maintenance cycle is the same from one maintenance cycle to the next maintenance cycle.

According to some embodiments of the methods of the invention, the duration of the maintenance cycle is from about 14 days to about 60 days, for example, from about 14 days to about 28 days, from about 21 days to about 42 days, from about 28 days to about 49 days, from about 28 days to about 56 days, or from about 21 days to about 28 days. In certain embodiments, the duration of the maintenance cycle is about 28 days. In some such embodiments, a therapeutic dose of the bispecific T-cell engaging molecule is administered by bolus intravenous infusion on days 1 and 15 of each maintenance cycle. In other embodiments in which the duration of the maintenance cycle is about 28 days, a therapeutic dose of the bispecific T-cell engaging molecule is administered by bolus intravenous infusion on days 1, 8, 15, and 22 of each maintenance cycle.

The methods described herein comprise administering to a patient a bispecific T-cell engaging molecule. The term “T-cell engaging molecule” refers to a molecule that comprises at least one domain in which the structure is derived from or comprises the minimum structural features of an antibody, e.g., of a full-length immunoglobulin molecule, that allow for specific binding to an antigen on the surface of a T cell, such as CD3. Thus, a T-cell engaging molecule according to the invention generally comprises one or more binding domains, each of which will typically comprise the minimum structural requirements of an antibody that allow for specific target binding. This minimum requirement may, for example, be defined by the presence of at least three light chain “complementarity determining regions” or CDRs (i.e. CDRL1, CDRL2 and CDRL3 of a VL region) and/or three heavy chain CDRs (i.e. CDRH1, CDRH2 and CDRH3 of a VH region), and preferably all six CDRs from both the light and heavy chain variable regions. The T-cell engaging molecules according to the invention may comprise domains or regions (e.g. CDRs or variable regions) from monoclonal, chimeric, humanized and human antibodies.

Preferably, the T-cell engaging molecules used in the methods of the invention are proteins and comprise one or more polypeptide chains. A polypeptide, as used herein, refers to a polymer of amino acids comprising at least 50 amino acids, preferably at least 100 amino acids. In some embodiments, the T-cell engaging molecules administered according to the methods of the invention are single-chain polypeptides. In other embodiments, the T-cell engaging molecules administered according to the methods of the invention comprise two or more polypeptide chains—e.g. are polypeptide dimers or multimers. In certain embodiments, the T-cell engaging molecules administered according to the methods of the invention comprise four polypeptide chains, and may, e.g. have the format of an antibody or an immunoglobulin protein.

As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein (e.g., target cancer cell antigen or CD3). From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

The T-cell engaging molecules used in the methods of the invention are preferably at least bispecific T-cell engaging molecules. The term “bispecific T-cell engaging molecule” refers to a molecule capable of specifically binding to two different antigens. In the context of the present invention, such bispecific T-cell engaging molecules specifically bind to a cancer cell antigen (e.g. human cancer cell antigen) on the cell surface of target cells and CD3 (e.g. human CD3) on the cell surface of T cells. In some embodiments, the T-cell engaging molecules may bind to more than one cancer cell antigen (e.g. human cancer cell antigen) on the cell surface of target cells as well as to CD3 (e.g. human CD3) on the cell surface of T cells. Thus, in such embodiments, the T-cell engaging molecules are “multitargeting” in that they are capable of specifically binding to two or more different cancer cell antigens and redirecting T cells to more than one type of cancer cell or cancer cells expressing the two or more antigens. A T-cell engaging molecule or binding domain thereof “specifically binds” to a target antigen when it has a significantly higher binding affinity for, and consequently is capable of distinguishing, that antigen compared to its affinity for other unrelated proteins, under similar binding assay conditions. T-cell engaging molecules or binding domains thereof that specifically bind an antigen may bind to that antigen with an equilibrium dissociation constant (K_{D})<1×10⁻⁶ M. T-cell engaging molecules or binding domains thereof specifically bind antigen with “high affinity” when the K_{D} is ≤1×10⁻⁸ M. In one embodiment, the T-cell engaging molecules or binding domains thereof used in the methods of the invention bind to a human cancer cell antigen and/or human CD3 with a K_{D} of ≤5×10⁻⁷ M. In another embodiment, the T-cell engaging molecules or binding domains thereof used in the methods of the invention bind to a human cancer cell antigen and/or human CD3 with a K_{D} of ≤1×10⁻⁷ M. In yet another embodiment, the T-cell engaging molecules or binding domains thereof used in the methods of the invention bind to a human cancer cell antigen and/or human CD3 with a K_{D} of ≤5×10⁻⁸ M. In another embodiment, the T-cell engaging molecules or binding domains thereof used in the methods of the invention bind to a human cancer cell antigen and/or human CD3 with a K_{D} of ≤2×10⁻⁸ M. In certain embodiments, the T-cell engaging molecules or binding domains thereof used in the methods of the invention bind to a human cancer cell antigen and/or human CD3 with a K_{D} of ≤1×10⁻⁸ M. In other embodiments, the T-cell engaging molecules or binding domains thereof used in the methods of the invention bind to a human cancer cell antigen and/or human CD3 with a K_{D} of ≤1×10⁻⁹ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and kₐ) and affinity (K_{D}) constants can be calculated in real-time using the bio-layer interferometry method. In some embodiments, the T-cell engaging molecules or binding domains thereof described herein exhibit desirable characteristics such as binding avidity as measured by k_{d} (dissociation rate constant) for a human cancer cell antigen and/or human CD3 of about 10⁻², 10⁻³, 10⁻⁴, 10⁻⁵, 10⁻⁶, 10⁻⁷, 10⁻¹, 10⁻¹, 10⁻¹⁰ s⁻¹ or lower (lower values indicating higher binding avidity), and/or binding affinity as measured by K_{D} (equilibrium dissociation constant) for a human cancer cell antigen and/or human CD3 of about 10⁻⁷, 10⁻¹, 10⁻⁹, 10⁻¹⁰, 10⁻¹¹ M or lower (lower values indicating higher binding affinity).

In some embodiments, bispecific T-cell engaging molecules used in the methods of the invention may be antibodies and have the general structure of a full-length immunoglobulin. For example, the bispecific T-cell engaging molecules may comprise two full-length antibody heavy chains and two full-length antibody light chains. In particular embodiments, the bispecific T-cell engaging molecules are heterodimeric antibodies (used interchangeably herein with “hetero immunoglobulins” or “hetero Igs”), which refer to antibodies comprising two different light chains and two different heavy chains. For instance, in some embodiments, the heterodimeric antibody comprises a light chain and heavy chain from an antibody that specifically binds to a cancer cell antigen, such as the cancer cell antigens described further herein, and a light chain and heavy chain from an antibody that specifically binds to CD3.

The bispecific T-cell engaging molecules employed in the methods of the invention may also comprise fragments of full-length antibodies, such as VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain). Bispecific T-cell engaging molecules according to the invention may also comprise modified fragments of antibodies. Examples of such modified fragments include, but are not limited to, single-chain variable fragment (scFv), di-scFv or bi(s)-scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH or VL, that specifically binds to an antigen or target independently of other variable regions or domains.

In certain embodiments, the bispecific T-cell engaging molecules used in the methods of the invention are multivalent. The valency of the T-cell engaging molecule denotes the number of individual antigen-binding domains within the T-cell engaging molecule. For example, the terms “monovalent,” “bivalent,” and “tetravalent” with reference to the T-cell engaging molecules in the context of the invention refer to T-cell engaging molecules with one, two, and four antigen-binding domains, respectively. Thus, a multivalent T-cell engaging molecule comprises two or more antigen-binding domains. A T-cell engaging molecule can have more antigen-binding domains (e.g. a higher valency) than specificities. For example, a T-cell engaging molecule having two antigen-binding domains for a first target (e.g. cancer cell antigen) and one antigen-binding domain for a second target (CD3)—or vice versa—is considered to be trivalent (three antigen-binding domains) and bispecific (binds to two antigens). In certain embodiments, the bispecific T-cell engaging molecules used in the methods of the invention are bivalent. Thus, such bispecific, bivalent T-cell engaging molecules contain two antigen binding domains: one antigen-binding domain for a cancer cell antigen (e.g. a human cancer cell antigen) and one antigen-binding domain for CD3 (e.g. human CD3). In other embodiments, the T-cell engaging molecules used in the methods of the invention are trivalent, trispecific T-cell engaging molecules and comprise three antigen binding domains: one antigen binding domain for a first cancer cell antigen, another antigen binding domain for a second cancer cell antigen, and a third binding domain for CD3. In still other embodiments, the T-cell engaging molecules used in the methods of the invention are tetravalent, trispecific T-cell engaging molecules and comprise four antigen binding domains: one antigen binding domain for a first cancer cell antigen, another antigen binding domain for a second cancer cell antigen, and two antigen binding domains for CD3.

In some embodiments, the bispecific T-cell engaging molecules employed in the methods of the invention comprise a first binding domain that specifically binds to a target cancer cell antigen (e.g. a human target cancer cell antigen) and a second binding domain that specifically binds to CD3 (e.g. human CD3). As used herein, the term “antigen-binding domain,” which is used interchangeably with “binding domain,” refers to the region of the T-cell engaging molecule that contains the amino acid residues that interact with the antigen and confer on the T-cell engaging molecule its specificity and affinity for the antigen. In certain embodiments, one or more binding domains of the T-cell engaging molecules may be derived from an antibody or antigen-binding fragment thereof. For instance, the binding domains of the bispecific T-cell engaging molecules used in the methods of the invention may comprise one or more CDRs from the light and heavy chain variable regions of antibodies that specifically bind to a human target cancer cell antigen and/or human CD3. In some embodiments, the anti-cancer cell antigen binding domain of the bispecific T-cell engaging molecules comprises all six CDRs of the heavy and light chain variable regions of an antibody that specifically binds to that human target cancer cell antigen and the anti-CD3 binding domain of the bispecific T-cell engaging molecules comprises all six CDRs of the heavy and light chain variable regions of an anti-CD3 antibody. In some embodiments, the binding domains (the anti-cancer cell antigen binding domain, the anti-CD3 binding domain or both) of the bispecific T-cell engaging molecules used in the methods of the invention comprise a Fab, a Fab′, a F(ab′)₂, a Fv, a single-chain variable fragment (scFv), or a nanobody. In one embodiment, both binding domains of the bispecific T-cell engaging molecule are Fab fragments. In another embodiment, one binding domain of the bispecific T-cell engaging molecule is a Fab fragment and the other binding domain is a scFv. In yet another embodiment, both binding domains of the bispecific T-cell engaging molecule are scFvs.

As used in the context of the invention, an “antigen-binding fragment,” used interchangeably herein with “binding fragment” or “fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)₂ fragment, a Fv fragment, a Fd fragment, and a CDR fragment, and can be derived from any mammalian source, such as human, mouse, rat, rabbit, or camelid. Antigen-binding fragments may compete for binding of a target antigen with an intact antibody and the fragments may be produced by the modification of intact antibodies (e.g. enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc domain” of an immunoglobulin generally comprises two constant domains, a CH2 domain and a CH3 domain, and optionally comprises a CH4 domain. In certain embodiments, the bispecific T-cell engaging molecules used in the methods of the invention comprise an Fc domain from an immunoglobulin. The Fc domain may be an Fc domain from an IgG1, IgG2, IgG3, or IgG4 immunoglobulin. In some embodiments, the Fc domain comprises CH2 and CH3 domains from a human IgG1 or human IgG2 immunoglobulin.

The Fc domain may retain effector function, such as Clq binding, complement dependent cytotoxicity (CDC), Fc receptor binding, antibody-dependent cell-mediated cytotoxicity (ADCC), and phagocytosis. In other embodiments, the Fc domain may be modified to reduce or eliminate effector function.

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H2L2 (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

In certain embodiments, the binding domains of the bispecific T-cell engaging molecules used in the methods of the invention comprise an immunoglobulin heavy chain variable region (VH) and an immunoglobulin light chain variable region (VL) of an antibody or antibody fragment which specifically binds to the desired antigen. For instance, the anti-cancer cell antigen binding domain of the bispecific T-cell engaging molecules of the invention comprises a VH region and VL region from an antibody that specifically binds to a target cancer cell antigen, such as any of the anti-cancer cell antigen antibodies or fragments thereof described herein, and the anti-CD3 binding domain comprises a VH region and VL region from an antibody that specifically binds to CD3, such as any of the anti-CD3 antibodies or fragments thereof described herein. The binding domains that specifically bind to a human cancer cell antigen or human CD3 can be derived from known antibodies to these antigens or from new antibodies or antibody fragments obtained by de novo immunization methods using the antigen proteins or fragments thereof, by phage display, or other methods known in the art. The antibodies from which the binding domains for the bispecific T-cell engaging molecules are derived can be monoclonal antibodies, recombinant antibodies, chimeric antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

The first binding domain of the bispecific T-cell engaging molecules used in the methods of the invention specifically binds to a target cancer cell antigen, preferably a human target cancer cell antigen. This binding domain is referred to herein as an anti-cancer cell antigen binding domain. The term “target cancer cell antigen” refers to an antigen expressed on the surface of a malignant cell, tumor cell, or other type of cancerous cell. A target cancer cell antigen may be expressed exclusively in cancer cells or may be overexpressed in cancer cells relative to normal cells. A target cancer cell antigen may also include a mutated or aberrant form of a protein expressed in cancer cells but not normal cells. Examples of a target cancer cell antigen include, but are not limited to, 5T4, AFP, BCMA, beta-catenin, BRCA1, CD19, CD20, CD22, CD33, CD70, CD123, CDH3, CDH19, CDK4, CEA, CLDN18.2, DLL3, DLL4, EGFR, EGFRvIII, EpCAM, EphA2, FLT3, FOLR1, gpA33, GPRC5D, HER2, IGFR, MAGE-1, MAGE-2, MAGE-3, MAGE-4, MAGE-6, MAGE-12, MSLN, MUC1, MUC2, MUC3, MUC4, MUC5, MUC16, MUC17, PSCA, PSMA, RAGE proteins, STEAP1, STEAP2, TRP1, and TRP2. In certain embodiments, the first domain of the bispecific T-cell engaging molecules used in the methods of the invention specifically binds to a target cancer cell antigen selected from MUC17, CLDN18.2, CD19, CD33, FLT3, DLL3, BCMA and PSMA.

In some embodiments, the first domain of the bispecific T-cell engaging molecules used in the methods of the invention specifically binds to CD19 (cluster of differentiation 19), preferably human CD19. Examples of anti-CD19 antibodies or binding domains from which the first binding domain of the bispecific T-cell engaging molecules used in the methods of the invention can be constructed or derived are described in, for example, WO 2010/052014, WO 2015/109131, WO 2017/134140, and WO 2020/018922, all of which are hereby incorporated by reference in their entireties. The anti-CD19 binding domain of the bispecific T-cell engaging molecules used in the methods of the invention may comprise an immunoglobulin heavy chain variable region (VH) and an immunoglobulin light chain variable region (VL) from an antibody that specifically binds to human CD19. The “variable region,” used interchangeably herein with “variable domain” (variable region of a light chain (VL), variable region of a heavy chain (VH)), refers to the region in each of the light and heavy immunoglobulin chains which is involved directly in binding of the antibody to the antigen. As discussed above, the regions of variable light and heavy chains have the same general structure and each region comprises four framework (FR) regions, the sequences of which are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site. Accordingly, in certain embodiments, the anti-CD19 binding domain of the bispecific T-cell engaging molecules suitable for use in the methods of the invention comprises a VH region comprising a CDRH1 having the sequence of SEQ ID NO: 1, a CDRH2 having the sequence of SEQ ID NO: 2, and a CDRH3 having the sequence of SEQ ID NO: 3, and a VL region comprising a CDRL1 having the sequence of SEQ ID NO: 5, a CDRL2 having the sequence of SEQ ID NO: 6, and a CDRL3 having the sequence of SEQ ID NO: 7. In some embodiments, the anti-CD19 binding domain of the bispecific T-cell engaging molecules comprises a VH region comprising (i) a sequence that is at least 90% identical to the sequence of SEQ ID NO: 4, (ii) a sequence that is at least 95% identical to the sequence of SEQ ID NO: 4, or (iii) the sequence of SEQ ID NO: 4. In these and other embodiments, the anti-CD19 binding domain of the bispecific T-cell engaging molecules comprises a VL region comprising (i) a sequence that is at least 90% identical to the sequence of SEQ ID NO: 8, (ii) a sequence that is at least 95% identical to the sequence of SEQ ID NO: 8, or (iii) the sequence of SEQ ID NO: 8. In one particular embodiment, the anti-CD19 binding domain of the bispecific T-cell engaging molecules for use in the methods of the invention comprises a VH region comprising the sequence of SEQ ID NO: 4 and a VL region comprising the sequence of SEQ ID NO: 8. In another particular embodiment, the anti-CD19 binding domain of the bispecific T-cell engaging molecules for use in the methods of the invention comprises the sequence of SEQ ID NO: 9.

In other embodiments, the first domain of the bispecific T-cell engaging molecules used in the methods of the invention specifically binds to CD33 (cluster of differentiation 33; also known as sialic acid binding Ig-like lectin 3 (SIGLEC3)), preferably human CD33. Examples of anti-CD33 antibodies or binding domains from which the first binding domain of the bispecific T-cell engaging molecules used in the methods of the invention can be constructed or derived are described in, for example, WO 2008/119567, WO 2012/045752, WO 2016/004108, WO 2017/134140, and WO 2019/224711, all of which are hereby incorporated by reference in their entireties. In some embodiments, the anti-CD33 binding domain of the bispecific T-cell engaging molecules suitable for use in the methods of the invention comprises a VH region comprising a CDRH1 having the sequence of SEQ ID NO: 11, a CDRH2 having the sequence of SEQ ID NO: 12, and a CDRH3 having the sequence of SEQ ID NO: 13, and a VL region comprising a CDRL1 having the sequence of SEQ ID NO: 15, a CDRL2 having the sequence of SEQ ID NO: 16, and a CDRL3 having the sequence of SEQ ID NO: 17. In related embodiments, the anti-CD33 binding domain of the bispecific T-cell engaging molecules comprises a VH region comprising (i) a sequence that is at least 90% identical to the sequence of SEQ ID NO: 14, (ii) a sequence that is at least 95% identical to the sequence of SEQ ID NO: 14, or (iii) the sequence of SEQ ID NO: 14. In these and other embodiments, the anti-CD33 binding domain of the bispecific T-cell engaging molecules comprises a VL region comprising (i) a sequence that is at least 90% identical to the sequence of SEQ ID NO: 18, (ii) a sequence that is at least 95% identical to the sequence of SEQ ID NO: 18, or (iii) the sequence of SEQ ID NO: 18. In certain embodiments, the anti-CD33 binding domain of the bispecific T-cell engaging molecules for use in the methods of the invention comprises a VH region comprising the sequence of SEQ ID NO: 14 and a VL region comprising the sequence of SEQ ID NO: 18. In certain other embodiments, the anti-CD33 binding domain of the bispecific T-cell engaging molecules for use in the methods of the invention comprises the sequence of SEQ ID NO: 19.

In still other embodiments, the first domain of the bispecific T-cell engaging molecules used in the methods of the invention specifically binds to FLT3 (fms-like tyrosine kinase 3; also known as cluster of differentiation antigen 135 (CD135)), preferably human FLT3. Examples of anti-FLT3 antibodies or binding domains from which the first binding domain of the bispecific T-cell engaging molecules used in the methods of the invention can be constructed or derived are described in, for example, WO 2017/021362 and WO 2017/134140, both of which are hereby incorporated by reference in their entireties. In some embodiments, the anti-FLT3 binding domain of the bispecific T-cell engaging molecules suitable for use in the methods of the invention comprises a VH region comprising a CDRH1 having the sequence of SEQ ID NO: 21, a CDRH2 having the sequence of SEQ ID NO: 22, and a CDRH3 having the sequence of SEQ ID NO: 23, and a VL region comprising a CDRL1 having the sequence of SEQ ID NO: 25, a CDRL2 having the sequence of SEQ ID NO: 26, and a CDRL3 having the sequence of SEQ ID NO: 27. In related embodiments, the anti-FLT3 binding domain of the bispecific T-cell engaging molecules comprises a VH region comprising (i) a sequence that is at least 90% identical to the sequence of SEQ ID NO: 24, (ii) a sequence that is at least 95% identical to the sequence of SEQ ID NO: 24, or (iii) the sequence of SEQ ID NO: 24. In these and other embodiments, the anti-FLT3 binding domain of the bispecific T-cell engaging molecules comprises a VL region comprising (i) a sequence that is at least 90% identical to the sequence of SEQ ID NO: 28, (ii) a sequence that is at least 95% identical to the sequence of SEQ ID NO: 28, or (iii) the sequence of SEQ ID NO: 28. In certain embodiments, the anti-FLT3 binding domain of the bispecific T-cell engaging molecules for use in the methods of the invention comprises a VH region comprising the sequence of SEQ ID NO: 24 and a VL region comprising the sequence of SEQ ID NO: 28. In certain other embodiments, the anti-FLT3 binding domain of the bispecific T-cell engaging molecules for use in the methods of the invention comprises the sequence of SEQ ID NO: 29.

In some embodiments, the first domain of the bispecific T-cell engaging molecules used in the methods of the invention specifically binds to DLL3 (delta-like ligand 3), preferably human DLL3. Examples of anti-DLL3 antibodies or binding domains from which the first binding domain of the bispecific T-cell engaging molecules used in the methods of the invention can be constructed or derived are described in, for example, WO 2013/126746, WO 2017/021349, WO 2017/134140, WO 2019/234220, and WO2020/069028, all of which are hereby incorporated by reference in their entireties. In some embodiments, the anti-DLL3 binding domain of the bispecific T-cell engaging molecules suitable for use in the methods of the invention comprises a VH region comprising a CDRH1 having the sequence of SEQ ID NO: 31, a CDRH2 having the sequence of SEQ ID NO: 32, and a CDRH3 having the sequence of SEQ ID NO: 33, and a VL region comprising a CDRL1 having the sequence of SEQ ID NO: 35, a CDRL2 having the sequence of SEQ ID NO: 36, and a CDRL3 having the sequence of SEQ ID NO: 37. In related embodiments, the anti-DLL3 binding domain of the bispecific T-cell engaging molecules comprises a VH region comprising (i) a sequence that is at least 90% identical to the sequence of SEQ ID NO: 34, (ii) a sequence that is at least 95% identical to the sequence of SEQ ID NO: 34, or (iii) the sequence of SEQ ID NO: 34. In these and other embodiments, the anti-DLL3 binding domain of the bispecific T-cell engaging molecules comprises a VL region comprising (i) a sequence that is at least 90% identical to the sequence of SEQ ID NO: 38, (ii) a sequence that is at least 95% identical to the sequence of SEQ ID NO: 38, or (iii) the sequence of SEQ ID NO: 38. In certain embodiments, the anti-DLL3 binding domain of the bispecific T-cell engaging molecules for use in the methods of the invention comprises a VH region comprising the sequence of SEQ ID NO: 34 and a VL region comprising the sequence of SEQ ID NO: 38. In certain other embodiments, the anti-DLL3 binding domain of the bispecific T-cell engaging molecules for use in the methods of the invention comprises the sequence of SEQ ID NO: 39.

In certain embodiments, the first domain of the bispecific T-cell engaging molecules used in the methods of the invention specifically binds to BCMA (B-cell maturation antigen), preferably human BCMA. Examples of anti-BCMA antibodies or binding domains from which the first binding domain of the bispecific T-cell engaging molecules used in the methods of the invention can be constructed or derived are described in, for example, WO 2013/072415, WO 2017/031104, WO 2017/134134, WO 2018/119215, WO 2019/075378, WO 2019/164891, and WO 2020/018820, all of which are hereby incorporated by reference in their entireties. In some embodiments, the anti-BCMA binding domain of the bispecific T-cell engaging molecules suitable for use in the methods of the invention comprises a VH region comprising a CDRH1 having the sequence of SEQ ID NO: 41, a CDRH2 having the sequence of SEQ ID NO: 42, and a CDRH3 having the sequence of SEQ ID NO: 43, and a VL region comprising a CDRL1 having the sequence of SEQ ID NO: 45, a CDRL2 having the sequence of SEQ ID NO: 46, and a CDRL3 having the sequence of SEQ ID NO: 47. In related embodiments, the anti-BCMA binding domain of the bispecific T-cell engaging molecules comprises a VH region comprising (i) a sequence that is at least 90% identical to the sequence of SEQ ID NO: 44, (ii) a sequence that is at least 95% identical to the sequence of SEQ ID NO: 44, or (iii) the sequence of SEQ ID NO: 44. In these and other embodiments, the anti-BCMA binding domain of the bispecific T-cell engaging molecules comprises a VL region comprising (i) a sequence that is at least 90% identical to the sequence of SEQ ID NO: 48, (ii) a sequence that is at least 95% identical to the sequence of SEQ ID NO: 48, or (iii) the sequence of SEQ ID NO: 48. In certain embodiments, the anti-BCMA binding domain of the bispecific T-cell engaging molecules for use in the methods of the invention comprises a VH region comprising the sequence of SEQ ID NO: 44 and a VL region comprising the sequence of SEQ ID NO: 48. In certain other embodiments, the anti-BCMA binding domain of the bispecific T-cell engaging molecules for use in the methods of the invention comprises the sequence of SEQ ID NO: 49.

In certain other embodiments, the first domain of the bispecific T-cell engaging molecules used in the methods of the invention specifically binds to PSMA (prostate-specific membrane antigen), preferably human PSMA. Examples of anti-PSMA antibodies or binding domains from which the first binding domain of the bispecific T-cell engaging molecules used in the methods of the invention can be constructed or derived are described in, for example, WO 2010/037836, WO 2017/023761, WO 2017/121905, WO 2017/134158, WO 2018/098356, WO 2019/092452, WO 2019/224718, and WO 2019/246514, all of which are hereby incorporated by reference in their entireties. In some embodiments, the anti-PSMA binding domain of the bispecific T-cell engaging molecules suitable for use in the methods of the invention comprises a VH region comprising a CDRH1 having the sequence of SEQ ID NO: 51, a CDRH2 having the sequence of SEQ ID NO: 52, and a CDRH3 having the sequence of SEQ ID NO: 53, and a VL region comprising a CDRL1 having the sequence of SEQ ID NO: 55, a CDRL2 having the sequence of SEQ ID NO: 56, and a CDRL3 having the sequence of SEQ ID NO: 57. In related embodiments, the anti-PSMA binding domain of the bispecific T-cell engaging molecules comprises a VH region comprising (i) a sequence that is at least 90% identical to the sequence of SEQ ID NO: 54, (ii) a sequence that is at least 95% identical to the sequence of SEQ ID NO: 54, or (iii) the sequence of SEQ ID NO: 54. In these and other embodiments, the anti-PSMA binding domain of the bispecific T-cell engaging molecules comprises a VL region comprising (i) a sequence that is at least 90% identical to the sequence of SEQ ID NO: 58, (ii) a sequence that is at least 95% identical to the sequence of SEQ ID NO: 58, or (iii) the sequence of SEQ ID NO: 58. In certain embodiments, the anti-PSMA binding domain of the bispecific T-cell engaging molecules for use in the methods of the invention comprises a VH region comprising the sequence of SEQ ID NO: 54 and a VL region comprising the sequence of SEQ ID NO: 58. In certain other embodiments, the anti-PSMA binding domain of the bispecific T-cell engaging molecules for use in the methods of the invention comprises the sequence of SEQ ID NO: 59.

In some embodiments, the first domain of the bispecific T-cell engaging molecules used in the methods of the invention specifically binds to CLDN18.2 (tight junction molecule claudin-18 isoform 2), preferably human CLDN18.2. Examples of anti-CLDN18.2 antibodies or binding domains from which the first binding domain of the bispecific T-cell engaging molecules used in the methods of the invention can be constructed or derived are described in, for example, WO 2007/059997, WO 2013/174509, WO 2014/127906, WO 2014/146778, WO 2014/075788, and WO 2020/025792, all of which are hereby incorporated by reference in their entireties. In some embodiments, the anti-CLDN18.2 binding domain of the bispecific T-cell engaging molecules suitable for use in the methods of the invention comprises a VH region comprising a CDRH1 having the sequence of SEQ ID NO: 149, a CDRH2 having the sequence of SEQ ID NO: 150, and a CDRH3 having the sequence of SEQ ID NO: 151, and a VL region comprising a CDRL1 having the sequence of SEQ ID NO: 154, a CDRL2 having the sequence of SEQ ID NO: 155, and a CDRL3 having the sequence of SEQ ID NO: 156. In related embodiments, the anti-CLDN18.2 binding domain of the bispecific T-cell engaging molecules comprises a VH region comprising (i) a sequence that is at least 90% identical to the sequence of SEQ ID NO: 152 or SEQ ID NO: 153, (ii) a sequence that is at least 95% identical to the sequence of SEQ ID NO: 152 or SEQ ID NO: 153, or (iii) the sequence of SEQ ID NO: 152 or SEQ ID NO: 153. In these and other embodiments, the anti-CLDN18.2 binding domain of the bispecific T-cell engaging molecules comprises a VL region comprising (i) a sequence that is at least 90% identical to the sequence of SEQ ID NO: 157, (ii) a sequence that is at least 95% identical to the sequence of SEQ ID NO: 157, or (iii) the sequence of SEQ ID NO: 157. In certain embodiments, the anti-CLDN18.2 binding domain of the bispecific T-cell engaging molecules for use in the methods of the invention comprises a VH region comprising the sequence of SEQ ID NO: 152 and a VL region comprising the sequence of SEQ ID NO: 157. In certain other embodiments, the anti-CLDN18.2 binding domain of the bispecific T-cell engaging molecules for use in the methods of the invention comprises a VH region comprising the sequence of SEQ ID NO: 153 and a VL region comprising the sequence of SEQ ID NO: 157. In some embodiments, the anti-CLDN18.2 binding domain of the bispecific T-cell engaging molecules for use in the methods of the invention comprises the sequence of SEQ ID NO: 158. In other embodiments, the anti-CLDN18.2 binding domain of the bispecific T-cell engaging molecules for use in the methods of the invention comprises the sequence of SEQ ID NO: 159.

In certain embodiments, the first domain of the bispecific T-cell engaging molecules used in the methods of the invention specifically binds to MUC17 (mucin 17), preferably human MUC17. Examples of anti-MUC17 antibodies or binding domains from which the first binding domain of the bispecific T-cell engaging molecules used in the methods of the invention can be constructed or derived are described in, for example, WO2019133961 and U.S. Pat. No. 8,546,546, both of which are hereby incorporated by reference in their entireties. In some embodiments, the anti-MUC17 binding domain of the bispecific T-cell engaging molecules suitable for use in the methods of the invention comprises a VH region comprising a CDRH1 having the sequence of SEQ ID NO: 162, a CDRH2 having the sequence of SEQ ID NO: 163, and a CDRH3 having the sequence of SEQ ID NO: 164, and a VL region comprising a CDRL1 having the sequence of SEQ ID NO: 166, a CDRL2 having the sequence of SEQ ID NO: 167, and a CDRL3 having the sequence of SEQ ID NO: 168. In related embodiments, the anti-MUC17 binding domain of the bispecific T-cell engaging molecules comprises a VH region comprising (i) a sequence that is at least 90% identical to the sequence of SEQ ID NO: 165, (ii) a sequence that is at least 95% identical to the sequence of SEQ ID NO: 165, or (iii) the sequence of SEQ ID NO: 165. In these and other embodiments, the anti-MUC17 binding domain of the bispecific T-cell engaging molecules comprises a VL region comprising (i) a sequence that is at least 90% identical to the sequence of SEQ ID NO: 169, (ii) a sequence that is at least 95% identical to the sequence of SEQ ID NO: 169, or (iii) the sequence of SEQ ID NO: 169. In certain embodiments, the anti-MUC17 binding domain of the bispecific T-cell engaging molecules for use in the methods of the invention comprises a VH region comprising the sequence of SEQ ID NO: 165 and a VL region comprising the sequence of SEQ ID NO: 169. In certain other embodiments, the anti-MUC17 binding domain of the bispecific T-cell engaging molecules for use in the methods of the invention comprises the sequence of SEQ ID NO: 170.

The term “identity,” as used herein, refers to a relationship between the sequences of two or more polypeptide molecules or two or more nucleic acid molecules, as determined by aligning and comparing the sequences. “Percent identity,” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (Dayhoff et al., in Atlas of Protein Sequence and Structure, vol. 5, supp. 3, 1978) or BLOSUM62 (Henikoff et al., 1992, Proc. Natl. Acad. Sci. U.S.A. 89:10915-10919) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, WI). The computer algorithm GAP is used to align the two polypeptides or two polynucleotides for which the percent sequence identity is to be determined. The sequences are aligned for optimal matching of their respective amino acid or nucleotide (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptide or nucleotide sequences using the GAP program include the following:
Algorithm: Needleman et al. 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The second binding domain of the bispecific T-cell engaging molecules used in the methods of the invention specifically binds to CD3, preferably human CD3. This binding domain is referred to herein as an anti-CD3 binding domain. “CD3” (cluster of differentiation 3) is a T cell co-receptor composed of four chains. In mammals, the CD3 protein complex contains a CD37 (gamma) chain, a CD36 (delta) chain, and two CD3F (epsilon) chains. These four chains associate with the T cell receptor (TCR) and the so-called ((zeta) chain to form the “T cell receptor complex” and to generate an activation signal in T lymphocytes. The CD37 (gamma), CD36 (delta), and CD3F (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide, which in humans is encoded by the CD3E gene which resides on chromosome 11.

The redirected lysis of target cells via the recruitment of T cells by a T-cell engaging molecule which binds to CD3 on the T cell and to a target protein (e.g. cancer cell antigen) on the target cell (e.g. tumor cell) generally involves cytolytic synapse formation and delivery of perforin and granzymes. The engaged T cells are capable of serial target cell lysis and are not affected by immune escape mechanisms interfering with peptide antigen processing and presentation, or clonal T cell differentiation; see, for example, WO 2007/042261.

In certain embodiments, the second binding domain of the bispecific T-cell engaging molecules used in the methods of the invention specifically binds to CD3 on the surface of a T cell, more preferably to human CD3 on the surface of a T cell. In some embodiments, the second binding domain of the bispecific T-cell engaging molecules specifically binds to CD3 epsilon, preferably human CD3 epsilon, e.g. human CD3 epsilon on the surface of a T cell. An exemplary amino acid sequence for the extracellular domain of human CD3 epsilon is set forth in SEQ ID NO: 61.

Examples of anti-CD3 antibodies or anti-CD3 binding domains from which the second binding domain of the bispecific T-cell engaging molecules used in the methods of the invention can be constructed or derived are described in WO 2007/042261, WO 2008/119567, WO 2017/053856, WO 2017/201493, WO 2017/223111, WO 2018/052503, and WO 2019/224717, all of which are hereby incorporated by reference in their entireties. In certain embodiments, the second domain of the bispecific T-cell engaging molecules used in the methods of the invention specifically binds to an epitope in the extracellular domain of human CD3 epsilon (e.g. an epitope within the polypeptide comprising the sequence of SEQ ID NO: 61). For instance, in some embodiments, the anti-CD3 binding domains of the bispecific T-cell engaging molecules suitable for use in the methods of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 82, 83 and 84, respectively, and CDRH1, CDRH2, and CDRH3 have the sequence of SEQ ID NOs: 62, 63 and 64, respectively;
(b) CDRL1, CDRL2, and CDRL3 have the sequence of SEQ ID NOs: 82, 83 and 84, respectively, and CDRH1, CDRH2, and CDRH3 have the sequence of SEQ ID NOs: 65, 66 and 67, respectively;
(c) CDRL1, CDRL2, and CDRL3 have the sequence of SEQ ID NOs: 82, 83 and 84, respectively, and CDRH1, CDRH2, and CDRH3 have the sequence of SEQ ID NOs: 68, 69 and 70, respectively;
(d) CDRL1, CDRL2, and CDRL3 have the sequence of SEQ ID NOs: 82, 83 and 84, respectively, and CDRH1, CDRH2, and CDRH3 have the sequence of SEQ ID NOs: 71, 69 and 72, respectively;
(e) CDRL1, CDRL2, and CDRL3 have the sequence of SEQ ID NOs: 85, 86 and 84, respectively, and CDRH1, CDRH2, and CDRH3 have the sequence of SEQ ID NOs: 74, 75 and 77, respectively;
(f) CDRL1, CDRL2, and CDRL3 have the sequence of SEQ ID NOs: 82, 83 and 84, respectively, and CDRH1, CDRH2, and CDRH3 have the sequence of SEQ ID NOs: 65, 63 and 73, respectively;
(g) CDRL1, CDRL2, and CDRL3 have the sequence of SEQ ID NOs: 85, 86 and 84, respectively, and CDRH1, CDRH2, and CDRH3 have the sequence of SEQ ID NOs: 78, 79 and 80, respectively;
(h) CDRL1, CDRL2, and CDRL3 have the sequence of SEQ ID NOs: 82, 83 and 84, respectively, and CDRH1, CDRH2, and CDRH3 have the sequence of SEQ ID NOs: 74, 75 and 76, respectively;
(i) CDRL1, CDRL2, and CDRL3 have the sequence of SEQ ID NOs: 87, 83 and 88, respectively, and CDRH1, CDRH2, and CDRH3 have the sequence of SEQ ID NOs: 68, 69 and 81, respectively; or
(j) CDRL1, CDRL2, and CDRL3 have the sequence of SEQ ID NOs: 87, 83 and 88, respectively, and CDRH1, CDRH2, and CDRH3 have the sequence of SEQ ID NOs: 65, 66 and 67, respectively. In a preferred embodiment, the anti-CD3 binding domain of the bispecific T-cell engaging molecules used in the methods of the invention comprises (i) a light chain variable region comprising a CDRL1 having the sequence of SEQ ID NO: 87, a CDRL2 having the sequence of SEQ ID NO: 83, and a CDRL3 having the sequence of SEQ ID NO: 88, and (ii) a heavy chain variable region comprising a CDRH1 having the sequence of SEQ ID NO: 65, a CDRH2 having the sequence of SEQ ID NO: 66, and a CDRH3 having the sequence of SEQ ID NO: 67.

The anti-CD3 binding domain of the bispecific T-cell engaging molecules according to the invention may comprise a light chain variable region comprising a sequence selected from SEQ ID NOs: 98-100 and/or a heavy chain variable region comprising a sequence selected from SEQ ID NOs: 89-97, and binding fragments, derivatives, and variants of these light chain and heavy chain variable regions. Each of the light chain variable regions set forth in SEQ ID NOs: 98-100 may be combined with any of the heavy chain variable regions set forth in SEQ ID NOs: 89-97 to form an anti-CD3 binding domain of the bispecific T-cell engaging molecules according to the invention. In certain embodiments, the anti-CD3 binding domains of the bispecific T-cell engaging molecules according to the invention comprise a light chain variable region comprising the sequence of SEQ ID NO: 98 and a heavy chain variable region comprising the sequence of SEQ ID NO: 89. In some embodiments, the anti-CD3 binding domains of the bispecific T-cell engaging molecules according to the invention comprise a light chain variable region comprising the sequence of SEQ ID NO: 98 and a heavy chain variable region comprising the sequence of SEQ ID NO: 90. In other embodiments, the anti-CD3 binding domains of the bispecific T-cell engaging molecules according to the invention comprise a light chain variable region comprising the sequence of SEQ ID NO: 98 and a heavy chain variable region comprising the sequence of SEQ ID NO: 91. In still other embodiments, the anti-CD3 binding domains of the bispecific T-cell engaging molecules according to the invention comprise a light chain variable region comprising the sequence of SEQ ID NO: 98 and a heavy chain variable region comprising the sequence of SEQ ID NO: 92. In some embodiments, the anti-CD3 binding domains of the bispecific T-cell engaging molecules according to the invention comprise a light chain variable region comprising the sequence of SEQ ID NO: 99 and a heavy chain variable region comprising the sequence of SEQ ID NO: 95.

In certain embodiments, the anti-CD3 binding domains of the bispecific T-cell engaging molecules according to the invention comprise a light chain variable region comprising the sequence of SEQ ID NO: 98 and a heavy chain variable region comprising the sequence of SEQ ID NO: 93. In one embodiment, the anti-CD3 binding domains of the bispecific T-cell engaging molecules according to the invention comprise a light chain variable region comprising the sequence of SEQ ID NO: 99 and a heavy chain variable region comprising the sequence of SEQ ID NO: 96. In another embodiment, the anti-CD3 binding domains of the bispecific T-cell engaging molecules according to the invention comprise a light chain variable region comprising the sequence of SEQ ID NO: 98 and a heavy chain variable region comprising the sequence of SEQ ID NO: 94. In a preferred embodiment, the anti-CD3 binding domains of the bispecific T-cell engaging molecules according to the invention comprise a light chain variable region comprising the sequence of SEQ ID NO: 100 and a heavy chain variable region comprising the sequence of SEQ ID NO: 90. In another preferred embodiment, the anti-CD3 binding domains of the bispecific T-cell engaging molecules according to the invention comprise a light chain variable region comprising the sequence of SEQ ID NO: 100 and a heavy chain variable region comprising the sequence of SEQ ID NO: 97.

In some embodiments, the anti-CD3 binding domains of the bispecific T-cell engaging molecules according to the invention comprise a light chain variable region comprising a sequence of contiguous amino acids that differs from the sequence of a light chain variable region set forth in SEQ ID NOs: 98-100 at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some anti-CD3 binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 98 to 100.

In one embodiment, the anti-CD3 binding domains of the bispecific T-cell engaging molecules according to the invention comprise a light chain variable region comprising a sequence that is at least 90% identical to a sequence selected from SEQ ID NOs: 98-100. In another embodiment, the anti-CD3 binding domains of the bispecific T-cell engaging molecules according to the invention comprise a light chain variable region comprising a sequence that is at least 95% identical to a sequence selected from SEQ ID NOs: 98-100. In yet another embodiment, the anti-CD3 binding domains of the bispecific T-cell engaging molecules according to the invention comprise a light chain variable region comprising a sequence selected from SEQ ID NOs: 98-100.

In these and other embodiments, the anti-CD3 binding domains of the bispecific T-cell engaging molecules according to the invention comprise a heavy chain variable region comprising a sequence of contiguous amino acids that differs from the sequence of a heavy chain variable region set forth in SEQ ID NOs: 89-97 at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some anti-CD3 binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 89 to 97.

In one embodiment, the anti-CD3 binding domains of the bispecific T-cell engaging molecules according to the invention comprise a heavy chain variable region comprising a sequence that is at least 90% identical to a sequence selected from SEQ ID NOs: 89-97. In another embodiment, the anti-CD3 binding domains of the bispecific T-cell engaging molecules according to the invention comprise a heavy chain variable region comprising a sequence that is at least 95% identical to a sequence selected from SEQ ID NOs: 89-97. In yet another embodiment, the anti-CD3 binding domains of the bispecific T-cell engaging molecules according to the invention comprise a heavy chain variable region comprising a sequence selected from SEQ ID NOs: 89-97.

According to certain embodiments, one or more of the binding domains of the bispecific T-cell engaging molecule used in the methods of the invention, are in the format of an scFv. In an scFv, the VH region and the VL region are arranged in the order VH-VL or VL-VH (from N- to C-terminus). It is envisaged that the VH and the VL regions of the first and/or the second binding domain are connected via a linker, preferably a peptide linker. In one embodiment of the first and/or second binding domain, the VH-region is positioned N-terminally of the linker, and the VL-region is positioned C-terminally of the linker. The linkers are preferably peptide linkers, more preferably short peptide linkers. Examples of suitable linkers include, but are not limited to, linkers comprising the sequences set forth in SEQ ID NOs: 111 to 124.

In the present context, a “short” linker has between 2 and 50 amino acids, preferably between 3 and 35, between 4 and 30, between 5 and 25, between 6 and 20 or between 6 and 17 amino acids. The linker between two variable regions of one binding domain may have a different length (e.g. may be longer) than the linker between the two binding domains. For example, the linker between two variable regions of one or both binding domains may have a length between 8 and 16 amino acids, preferably between 10 and 15, and the linker between the two binding domains may have a length between 3 and 10 amino acids, preferably between 5 and 8. It is further envisaged that the peptide linkers are glycine/serine linkers, such as those depicted in SEQ ID NOs: 112-116 and 118-124. In one embodiment, the anti-cancer cell antigen binding domain and/or the anti-CD3 binding domain of the bispecific T-cell engaging molecule according to the invention is an scFv comprising, from N-terminus to C-terminus, a VH region-peptide linker—VL region, where the peptide linker comprises a glycine-serine linker, such as the linker set forth in SEQ ID NO: 119. In another embodiment, the anti-cancer cell antigen binding domain and/or the anti-CD3 binding domain of the bispecific T-cell engaging molecule according to the invention is an scFv comprising, from N-terminus to C-terminus, a VL region-peptide linker—VH region, where the peptide linker comprises a glycine-serine linker, such as the linker set forth in SEQ ID NO: 119. In related embodiments, the peptide linker between the anti-cancer cell antigen binding domain and anti-CD3 binding domain (e.g. scFv domains) is the linker set forth in SEQ ID NO: 112 or SEQ ID NO: 115. In certain embodiments, the anti-cancer cell antigen binding domain of the bispecific T-cell engaging molecules is an scFv domain and comprises a sequence selected from SEQ ID NO: 9, SEQ ID NO: 19, SEQ ID NO: 29, SEQ ID NO: 39, SEQ ID NO: 49, SEQ ID NO: 59, SEQ ID NO: 158, SEQ ID NO: 159, and SEQ ID NO: 170. In these and other embodiments, the anti-CD3 binding domain of the bispecific T-cell engaging molecules is an scFv domain and comprises a sequence selected from SEQ ID NOs: 101-110.

In certain embodiments, the bispecific T-cell engaging molecules suitable for use in the methods of the invention comprise a first binding domain that specifically binds to a human target cancer cell antigen and has an amino acid sequence selected from any one of SEQ ID NO: 9, SEQ ID NO: 19, SEQ ID NO: 29, SEQ ID NO: 39, SEQ ID NO: 49, SEQ ID NO: 59, SEQ ID NO: 158, SEQ ID NO: 159, and SEQ ID NO: 170, and a second binding domain that specifically binds to human CD3 and has an amino acid sequence selected from any one of SEQ ID NOs: 101-110. In a preferred embodiment, the first binding domain (e.g. anti-cancer cell antigen binding domain) of the bispecific T-cell engaging molecules comprises the amino acid sequence of SEQ ID NO: 49 and the second binding domain (e.g. the anti-CD3 binding domain) of the bispecific T-cell engaging molecules comprises the amino acid sequence of SEQ ID NO: 110. In another preferred embodiment, the first binding domain (e.g. anti-cancer cell antigen binding domain) of the bispecific T-cell engaging molecules comprises the amino acid sequence of SEQ ID NO: 59 and the second binding domain (e.g. the anti-CD3 binding domain) of the bispecific T-cell engaging molecules comprises the amino acid sequence of SEQ ID NO: 110.

The bispecific T-cell engaging molecules suitable for use in the methods of the invention can comprise any of the anti-cancer cell antigen scFv binding domains set forth in SEQ ID NO: 9, SEQ ID NO: 19, SEQ ID NO: 29, SEQ ID NO: 39, SEQ ID NO: 49, SEQ ID NO: 59, SEQ ID NO: 158, SEQ ID NO: 159, and SEQ ID NO: 170 in combination with any of the anti-CD3 scFv binding domains set forth in SEQ ID NOs: 101-110. For instance, in some embodiments, the bispecific T-cell engaging molecules comprise an anti-cancer cell antigen scFv binding domain set forth in SEQ ID NO: 9, SEQ ID NO: 19, SEQ ID NO: 29, SEQ ID NO: 39, SEQ ID NO: 49, SEQ ID NO: 59, SEQ ID NO: 158, SEQ ID NO: 159, or SEQ ID NO: 170 and an anti-CD3 scFv binding domain set forth in SEQ ID NOs: 101-110, wherein the anti-cancer cell antigen scFv binding domain is connected to the anti-CD3 scFv binding domain through a peptide linker, such as the peptide linkers described herein. In certain embodiments, the bispecific T-cell engaging molecule comprises, in amino to carboxyl order, an anti-cancer cell antigen scFv binding domain, a peptide linker, and an anti-CD3 scFv binding domain. In some such embodiments, the peptide linker comprises the sequence of SEQ ID NO: 112 or SEQ ID NO: 115.

The bispecific T-cell engaging molecules suitable for use in the methods of the invention preferably comprise additional domains, which, e.g., can modulate the pharmacokinetic profile of the molecule. For instance, the bispecific T-cell engaging molecules may further comprise a domain or moiety that increases the elimination half-life of the molecule. The elimination half-life refers to the time it takes for the concentration of a drug in the plasma or the total amount in the body to be reduced by 50%. Thus, after one half-life, the concentration of the drug in the body will be half of the starting dose. Preferably, the bispecific T-cell engaging molecules comprise a half-life extension moiety that provides a half-life for the molecule of greater than 24 hours, greater than 48 hours, greater than 72 hours, greater than 5 days, greater than 7 days, greater than 10 days, greater than 14 days, or greater than 21 days. Accordingly, the bispecific T-cell engaging molecules suitable for use in the methods of the invention may have a half-life of about 2 days to about 21 days, about 3 days to about 14 days, about 5 days to about 15 days, about 3 days to about 7 days, or about 2 days to about 5 days. Examples of half-life extension moieties that can be incorporated into the bispecific T-cell engaging molecules used in the methods of the invention can include, but are not limited to, an immunoglobulin Fc domain, a domain derived from serum albumin (e.g. human serum albumin), or an albumin-binding domain (e.g. comprising human albumin binding peptides), peptides that bind to the neonatal Fc receptor (FcRn), and polyethylene glycol polymers. Examples of domains derived from human serum albumin or variants thereof that can be incorporated into the bispecific T-cell engaging molecules are described, for example, in WO 2011/051489, WO 2012/059486, WO 2013/075066, WO 2013/135896, and WO 2014/072481, all of which are hereby incorporated by reference in their entireties. In some embodiments, the half-life extension moiety incorporated into the bispecific T-cell engaging molecules used in the methods of the invention is an albumin-binding domain, such as a domain comprising an albumin-binding peptide or an antibody fragment (e.g. single domain antibodies or scFv domains) that specifically binds to serum albumin. Examples of albumin-binding domains that may be incorporated into the bispecific T-cell engaging molecules suitable for use in the methods of the invention are described in, for example, WO 2013/128027, WO 2014/140358, and WO 2017201488, all of which are hereby incorporated by reference in their entireties.

In certain embodiments, the bispecific T-cell engaging molecules used in the methods of the invention comprise an immunoglobulin Fc domain. The immunoglobulin Fc domain may comprise one or more Fc monomers. Each “Fc monomer” typically comprises at least a CH2 domain and a CH3 domain from an immunoglobulin molecule. The Fc monomer may comprise the CH2 and CH3 domains from an IgG1, IgG2, IgG3, or IgG4 immunoglobulin. As an example, the CH2 domain comprises amino acids 231 to 340 of an IgG1 immunoglobulin and the CH3 domain comprises amino acids 341 to 446 of an IgG1 immunoglobulin, where the amino acid numbering is according to the EU numbering system described in Edelman et al., Proc. Natl. Acad. USA, Vol. 63: 78-85 (1969) and Kabat et al., Sequences of Proteins of Immunological Interest, 5th Ed. Public Health Service, National Institutes of Health Publication No. 91-3242, Bethesda, MD (1991). The boundaries of the CH2 and CH3 domains may vary slightly from one IgG isoform to another, but the CH2 and CH3 domains in IgG2, IgG3, and IgG4 can be ascertained by alignment with the CH2 and CH3 domains in IgG1.

In some embodiments, the Fc monomer may comprise an immunoglobulin hinge region or portion thereof. The immunoglobulin hinge region is typically the region defined by amino acids 216 to 231 (according to the EU numbering system) of IgG immunoglobulins. In certain embodiments, the Fc monomer comprises a hinge region from an IgG1 immunoglobulin or a portion thereof. In some such embodiments, the IgG1 hinge region comprises the amino acid sequence DKTHTCPPCP (SEQ ID NO: 125) or EPKSCDKTHTCPPCP (SEQ ID NO: 126). In other embodiments, the Fc monomer comprises an IgG2 hinge region having the sequence ERKCCVECPPCP (SEQ ID NO: 127), an IgG3 hinge region having the sequence ELKTPLDTTHTCPRCP (SEQ ID NO: 128), EPKSCDTPPPCPRCP (SEQ ID NO: 129), or ELKTPLGDTTHTCPRCP (SEQ ID NO: 130), or an IgG4 hinge region having the sequence ESKYGPPCPSCP (SEQ ID NO: 131). In certain embodiments, the Fc monomer comprises, in amino to carboxyl order, an immunoglobulin hinge region, an immunoglobulin CH2 domain, and an immunoglobulin CH3 domain.

In certain embodiments, the bispecific T-cell engaging molecules comprise an Fc domain having one Fc monomer. In alternative embodiments, the bispecific T-cell engaging molecules comprise an Fc domain having two or more Fc monomers. For instance, in one embodiment, the bispecific T-cell engaging molecules used in the methods of the invention comprise an Fc domain having two Fc monomers. The two Fc monomers can be present on separate polypeptide chains and associate to form a dimer, e.g. via non-covalent interactions and/or disulfide bonds (e.g. between cysteine residues in the hinge regions of Fc monomers). In another embodiment, the two Fc monomers are fused to each other via a peptide linker, preferably a linker sufficient in length to allow the Fc monomers to associate and form an intra-chain dimer. The fusion of two Fc monomers to form a single polypeptide chain is referred to herein as a single-chain Fc domain (scFc domain) and is described in more detail below.

The peptide linker, by which the Fc monomers are fused to each other to form a single-chain Fc domain, preferably comprises at least 25 amino acid residues (e.g. 25, 26, 27, 28, 29, 30 or more). More preferably, this peptide linker comprises at least 30 amino acid residues (e.g. 30, 31, 32, 33, 34, 35 or more). In some embodiments, the linker comprises up to 40 amino acid residues, more preferably up to 35 amino acid residues, and even more preferably exactly 30 amino acid residues. In certain embodiments, the peptide linker comprises glycine-serine residues, for example repeats of the amino acid sequence Gly-Gly-Gly-Gly-Ser (SEQ ID NO: 112). In such embodiments, the peptide linker comprises (Gly₄Ser)ₓ, where x is an integer of 5 or greater (e.g. 6, 7 or 8). Preferably the integer is 6 or 7, more preferably the integer is 6. In one particular embodiment, the peptide linker used to connect the two Fc monomers to form a single-chain Fe domain comprises the sequence of SEQ ID NO: 122.

The Fc monomer may contain one or more amino acid substitutions relative to the native CH2 or CH3 immunoglobulin amino acid sequences, e.g. to modulate effector function, alter glycosylation, or enhance stability. For instance, in one embodiment, the glycosylation site in the CH2 domain at amino acid position 297 according to EU numbering is removed by substituting a different amino acid for the asparagine residue at this position. A N297G substitution is preferred in some embodiments. Stability-enhancing mutations include the substitution of one or more amino acids in the CH2 and/or CH3 domains with cysteine residues to promote disulfide bond formation. Preferably, specific pairs of residues are substituted with cysteine such that they preferentially form a disulfide bond with each other, thus limiting or preventing disulfide bond scrambling. Preferred pairs include, but are not limited to, A287C and L306C, V259C and L306C, R292C and V302C, and V323C and I332C, with the amino acid positions numbered according to the EU numbering system. In one particular embodiment, the Fc monomer(s) incorporated into the Fc domain of the bispecific T-cell engaging molecules comprises N297G, R292C, and V302C substitutions, with the amino acid positions numbered according to the EU numbering system.

In certain embodiments, the bispecific T-cell engaging molecules used in the methods of the invention comprise an Fc domain, which is a single-chain Fc domain. Accordingly, in certain such embodiments, the Fc domain comprises two Fc monomers, each monomer comprising an immunoglobulin hinge region, an immunoglobulin CH2 domain, and an immunoglobulin CH3 domain, wherein the two Fc monomers are fused to each other via a peptide linker as described herein. Exemplary amino acid sequences for the Fc monomers are set forth in SEQ ID NOs: 132-139 and exemplary amino acid sequences for the single-chain Fc (scFc) domains are set forth in SEQ ID NOs: 140-148. In some embodiments, each of the Fc monomers of the Fc domain has an amino acid sequence that is at least 90% identical to a sequence selected from SEQ ID NOs: 132-139. In other embodiments, each of the Fc monomers of the Fc domain has an amino acid sequence selected from SEQ ID NOs: 132-139. In a preferred embodiment, each of the Fc monomers of the Fc domain comprises the amino acid sequence of SEQ ID NO: 132. In another preferred embodiment, each of the Fc monomers of the Fc domain comprises the amino acid sequence of SEQ ID NO: 133.

The Fe domain of the bispecific T-cell engaging molecules used in the methods of the invention can comprise the sequences of any of the scFc domains set forth in SEQ ID NOs: 140-148 or a variant of these scFc domains. In one embodiment, the bispecific T-cell engaging molecules according to the invention comprise an Fc domain comprising an amino acid sequence that is at least 90% identical to a sequence selected from SEQ ID NOs: 140-148. In another embodiment, the bispecific T-cell engaging molecules according to the invention comprise an Fc domain comprising an amino acid sequence selected from SEQ ID NOs: 140-148. In a preferred embodiment, the bispecific T-cell engaging molecules according to the invention comprise an Fc domain comprising the amino acid sequence of SEQ ID NO: 140. In another preferred embodiment, the bispecific T-cell engaging molecules according to the invention comprise an Fc domain comprising the amino acid sequence of SEQ ID NO: 141. In yet another preferred embodiment, the bispecific T-cell engaging molecules according to the invention comprise an Fc domain comprising the amino acid sequence of SEQ ID NO: 148.

In certain embodiments, the bispecific T-cell engaging molecules used in the methods of the invention comprise, in an amino to carboxyl order:
(i) a first domain that specifically binds to a target cancer cell antigen (e.g. a human cancer cell antigen) comprising a first immunoglobulin heavy chain variable region (VH1) and a first immunoglobulin light chain variable region (VL1);
(ii) a second domain that specifically binds to CD3 (e.g. human CD3) comprising a second immunoglobulin heavy chain variable region (VH2) and a second immunoglobulin light chain variable region (VL2); and
(iii) an Fc domain comprising two Fc monomers.

In some embodiments, the bispecific T-cell engaging molecules comprise, in amino to carboxyl order:
(i) a first domain that specifically binds to a target cancer cell antigen comprising a VH1 comprising a CDRH1, a CDRH2, and a CDRH3, and a VL1 comprising a CDRL1, a CDRL2, and a CDRL3, wherein:
   (a) CDRH1, CDRH2 and CDRH3 have the sequence of SEQ ID NOs: 1, 2, and 3, respectively, and CDRL1, CDRL2, and CDRL3 have the sequence of SEQ ID NOs: 5, 6, and 7, respectively;
   (b) CDRH1, CDRH2 and CDRH3 have the sequence of SEQ ID NOs: 11, 12, and 13, respectively, and CDRL1, CDRL2, and CDRL3 have the sequence of SEQ ID NOs: 15, 16, and 17, respectively;
   (c) CDRH1, CDRH2 and CDRH3 have the sequence of SEQ ID NOs: 21, 22, and 23, respectively, and CDRL1, CDRL2, and CDRL3 have the sequence of SEQ ID NOs: 25, 26, and 27, respectively;
   (d) CDRH1, CDRH2 and CDRH3 have the sequence of SEQ ID NOs: 31, 32, and 33, respectively, and CDRL1, CDRL2, and CDRL3 have the sequence of SEQ ID NOs: 35, 36, and 37, respectively;
   (e) CDRH1, CDRH2 and CDRH3 have the sequence of SEQ ID NOs: 41, 42, and 43, respectively, and CDRL1, CDRL2, and CDRL3 have the sequence of SEQ ID NOs: 45, 46, and 47, respectively;
   (f) CDRH1, CDRH2 and CDRH3 have the sequence of SEQ ID NOs: 51, 52, and 53, respectively, and CDRL1, CDRL2, and CDRL3 have the sequence of SEQ ID NOs: 55, 56, and 57, respectively;
   (g) CDRH1, CDRH2 and CDRH3 have the sequence of SEQ ID NOs: 149, 150, and 151, respectively, and CDRL1, CDRL2, and CDRL3 have the sequence of SEQ ID NOs: 154, 155, and 156, respectively; or
   (h) CDRH1, CDRH2 and CDRH3 have the sequence of SEQ ID NOs: 162, 163, and 164, respectively, and CDRL1, CDRL2, and CDRL3 have the sequence of SEQ ID NOs: 166, 167, and 168, respectively;
(ii) a second domain that specifically binds to human CD3 comprising a VH2 comprising a CDRH1 having the sequence of SEQ ID NO: 65, a CDRH2 having the sequence of SEQ ID NO: 66, and a CDRH3 having the sequence of SEQ ID NO: 67, and a VL2 comprising a CDRL1 having the sequence of SEQ ID NO: 87, a CDRL2 having the sequence of SEQ ID NO: 83, and a CDRL3 having the sequence of SEQ ID NO: 88; and
(iii) an Fc domain comprising two Fc monomers, each monomer comprising an immunoglobulin hinge region, a CH2 domain, and a CH3 domain, wherein said two monomers are fused to each other via a peptide linker.

In related embodiments, the bispecific T-cell engaging molecules comprise, in amino to carboxyl order:
(i) a first domain that specifically binds to a target cancer cell antigen comprising a VH1 and a VL1, wherein:
   (a) VH1 comprises the sequence of SEQ ID NO: 4 and VL1 comprises the sequence of SEQ ID NO: 8;
   (b) VH1 comprises the sequence of SEQ ID NO: 14 and VL1 comprises the sequence of SEQ ID NO: 18;
   (c) VH1 comprises the sequence of SEQ ID NO: 24 and VL1 comprises the sequence of SEQ ID NO: 28;
   (d) VH1 comprises the sequence of SEQ ID NO: 34 and VL1 comprises the sequence of SEQ ID NO: 38;
   (e) VH1 comprises the sequence of SEQ ID NO: 44 and VL1 comprises the sequence of SEQ ID NO: 48;
   (f) VH1 comprises the sequence of SEQ ID NO: 54 and VL1 comprises the sequence of SEQ ID NO: 58;
   (g) VH1 comprises the sequence of SEQ ID NO: 152 and VL1 comprises the sequence of SEQ ID NO: 157;
   (h) VH1 comprises the sequence of SEQ ID NO: 153 and VL1 comprises the sequence of SEQ ID NO: 157; or
   (i) VH1 comprises the sequence of SEQ ID NO: 165 and VL1 comprises the sequence of SEQ ID NO: 169;
(ii) a second domain that specifically binds to human CD3 comprising a VH2 comprising the sequence of SEQ ID NO: 90 and a VL2 comprising the sequence of SEQ ID NO: 100; and
(iii) an Fc domain comprising two Fc monomers, each monomer comprising an immunoglobulin hinge region, a CH2 domain, and a CH3 domain, wherein said two monomers are fused to each other via a peptide linker.

In certain embodiments, peptide linkers, such as those described herein, connect the first domain to the second domain and/or the second domain to the Fc domain. Accordingly, in some embodiments, the bispecific T-cell engaging molecule according to the invention comprises, in amino to carboxyl order:
(i) a first domain that specifically binds to a target cancer cell antigen (e.g. a human cancer cell antigen);
(ii) a first peptide linker having an amino acid sequence selected from SEQ ID NOs: 112, 115, 118, and 119;
(iii) a second domain that specifically binds to CD3 (e.g. human CD3);
(iv) a second peptide linker having an amino acid sequence selected from SEQ ID NOs: 111-115, 118, and 119;
(v) a first Fc monomer;
(vi) a third peptide linker having an amino acid sequence selected from SEQ ID NOs: 121-124; and
(vii) a second Fc monomer.

In other embodiments, the bispecific T-cell engaging molecule according to the invention comprises, in amino to carboxyl order:
(i) a first domain (e.g. anti-cancer cell antigen binding domain) having an amino acid sequence selected from SEQ ID NO: 9, SEQ ID NO: 19, SEQ ID NO: 29, SEQ ID NO: 39, SEQ ID NO: 49, SEQ ID NO: 59, SEQ ID NO: 158, SEQ ID NO: 159, and SEQ ID NO: 170;
(ii) a first peptide linker having an amino acid sequence selected from SEQ ID NOs: 112, 115, 118, and 119;
(iii) a second domain (e.g. anti-CD3 binding domain) having an amino acid sequence selected from SEQ ID NOs: 101-110;
(iv) a second peptide linker having an amino acid sequence selected from SEQ ID NOs: SEQ ID NOs: 111-115, 118, and 119;
(v) a first Fc monomer having an amino acid sequence selected from SEQ ID NOs: 132-139;
(vi) a third peptide linker having an amino acid sequence selected from SEQ ID NOs: SEQ ID NOs: 121-124; and
(vii) a second Fc monomer having an amino acid sequence selected from SEQ ID NOs: 132-139.

In some embodiments, the bispecific T-cell engaging molecule according to the invention comprises, in amino to carboxyl order:
(i) a first domain (e.g. anti-cancer cell antigen binding domain) having an amino acid sequence selected from SEQ ID NO: 9, SEQ ID NO: 19, SEQ ID NO: 29, SEQ ID NO: 39, SEQ ID NO: 49, SEQ ID NO: 59, SEQ ID NO: 158, SEQ ID NO: 159, and SEQ ID NO: 170;
(ii) a first peptide linker having the amino acid sequence of SEQ ID NO: 112 or SEQ ID NO: 115;
(iii) a second domain (e.g. anti-CD3 binding domain) having the amino acid sequence of SEQ ID NO: 110;
(iv) a second peptide linker having the amino acid sequence of SEQ ID NO: 111 or SEQ ID NO: 112;
(v) a first Fc monomer having the amino acid sequence of SEQ ID NO: 132;
(vi) a third peptide linker having the amino acid sequence of SEQ ID NO: 122 or SEQ ID NO: 123; and
(vii) a second Fc monomer having the amino acid sequence of SEQ ID NO: 132.

In certain embodiments, the bispecific T-cell engaging molecules used in the methods of the invention are single chain polypeptides or single chain fusion proteins. As used herein, a “single chain polypeptide” or “single chain fusion protein” refers to a molecule consisting of only one polypeptide chain, i.e. all of the domains in the bispecific T-cell engaging molecule are linked together, optionally via peptide linkers, to form a single polypeptide chain. One example of such a single chain polypeptide or single chain fusion protein in the context of the present invention is a single chain polypeptide comprising, in an amino to carboxyl order, an anti-cancer cell antigen scFv domain, a first peptide linker, an anti-CD3 scFv domain, a second peptide linker, and an scFc domain. Exemplary bispecific single chain polypeptides or single chain fusion proteins that can be used in the methods of the invention are set forth in SEQ ID NO: 10, SEQ ID NO: 20, SEQ ID NO: 30, SEQ ID NO: 40, SEQ ID NO: 50, SEQ ID NO: 60, SEQ ID NO: 160, SEQ ID NO: 161, and SEQ ID NO: 171. Other bispecific single chain polypeptides or single chain fusion proteins suitable for use in the methods of the invention are described in WO 2017/021362, WO 2017/021349, WO 2017/134134, WO 2017/134140, WO 2017/134158, WO 2019/133961, and WO 2020/025792, all of which are hereby incorporated by reference in their entireties.

In some embodiments, the bispecific T-cell engaging molecule administered to a patient according to the methods of the invention comprises an amino acid sequence selected from SEQ ID NO: 10, SEQ ID NO: 20, SEQ ID NO: 30, SEQ ID NO: 40, SEQ ID NO: 50, SEQ ID NO: 60, SEQ ID NO: 160, SEQ ID NO: 161, and SEQ ID NO: 171 or a variant of one of these sequences. For example, the bispecific T-cell engaging molecule employed in the methods of the invention may comprise an amino acid sequence that is at least 90%, 91%, 92%, 93%, 94%, 95%, 96%, 97%, 98% or 99% identical to any of SEQ ID NO: 10, SEQ ID NO: 20, SEQ ID NO: 30, SEQ ID NO: 40, SEQ ID NO: 50, SEQ ID NO: 60, SEQ ID NO: 160, SEQ ID NO: 161, or SEQ ID NO: 171. In some such embodiments, the sequence variability occurs in the peptide linker regions and/or the single-chain Fc domain.

In one embodiment, the patient to be treated according to the methods of the invention is diagnosed with or has leukemia or lymphoma, such as diffuse large B-cell lymphoma, Burkitt lymphoma, follicular lymphoma, Non-Hodgkin lymphoma, or acute lymphoblastic leukemia, and the anti-cancer cell antigen binding domain of the bispecific T-cell engaging molecule specifically binds to CD19. Any of the bispecific T-cell engaging molecules comprising an anti-CD19 binding domain described herein can be administered to such a patient according to the methods of the invention. In certain embodiments, the bispecific T-cell engaging molecule administered according to the methods of the invention to a patient diagnosed with or having leukemia or lymphoma is a single chain polypeptide comprising the sequence of SEQ ID NO: 10.

In another embodiment, the patient to be treated according to the methods of the invention is diagnosed with myeloid leukemia, particularly acute myeloid leukemia, and the anti-cancer cell antigen binding domain of the bispecific T-cell engaging molecule specifically binds to CD33 or FLT3. Any of the bispecific T-cell engaging molecules comprising an anti-CD33 binding domain or an anti-FLT3 binding domain described herein can be administered to such a patient according to the methods of the invention. In certain embodiments, the bispecific T-cell engaging molecule administered according to the methods of the invention to a patient diagnosed with or having myeloid leukemia is a single chain polypeptide comprising the sequence of SEQ ID NO: 20. In other embodiments, the bispecific T-cell engaging molecule administered according to the methods of the invention to a patient diagnosed with or having myeloid leukemia is a single chain polypeptide comprising the sequence of SEQ ID NO: 30.

In yet another embodiment, the patient to be treated according to the methods of the invention is diagnosed with or has a DLL3-expressing cancer, such as small-cell lung cancer, neuroendocrine prostate cancer, melanoma, or glioblastoma, and the anti-cancer cell antigen binding domain of the bispecific T-cell engaging molecule specifically binds to DLL3. Any of the bispecific T-cell engaging molecules comprising an anti-DLL3 binding domain described herein can be administered to such a patient according to the methods of the invention. In certain embodiments, the bispecific T-cell engaging molecule administered according to the methods of the invention to a patient diagnosed with or having a DLL3-expressing cancer (e.g. small-cell lung cancer) is a single chain polypeptide comprising the sequence of SEQ ID NO: 40.

In certain embodiments, the patient to be treated according to the methods of the invention is diagnosed with or has multiple myeloma, and the anti-cancer cell antigen binding domain of the bispecific T-cell engaging molecule specifically binds to BCMA. Any of the bispecific T-cell engaging molecules comprising an anti-BCMA binding domain described herein can be administered to such a patient according to the methods of the invention. In certain embodiments, the bispecific T-cell engaging molecule administered according to the methods of the invention to a patient diagnosed with or having multiple myeloma is a single chain polypeptide comprising the sequence of SEQ ID NO: 50.

In certain other embodiments, the patient to be treated according to the methods of the invention is diagnosed with or has a PSMA-expressing cancer, such as prostate cancer, non-small cell lung cancer, small cell lung cancer, renal cell carcinoma, hepatocellular carcinoma, bladder cancer, testicular cancer, colon cancer, glioblastoma, breast cancer, ovarian cancer, endometrial cancer, or melanoma, and the anti-cancer cell antigen binding domain of the bispecific T-cell engaging molecule specifically binds to PSMA. Any of the bispecific T-cell engaging molecules comprising an anti-PSMA binding domain described herein can be administered to such a patient according to the methods of the invention. In certain embodiments, the bispecific T-cell engaging molecule administered according to the methods of the invention to a patient diagnosed with or having a PSMA-expressing cancer (e.g. prostate cancer) is a single chain polypeptide comprising the sequence of SEQ ID NO: 60.

In some embodiments, the patient to be treated according to the methods of the invention is diagnosed with a CLDN18.2-expressing cancer, such as colorectal cancer, pancreatic cancer, ovarian cancer, lung cancer, and gastrointestinal cancer, particularly gastric cancer, esophageal cancer, and gastroesophageal junction cancer, and the anti-cancer cell antigen binding domain of the bispecific T-cell engaging molecule specifically binds to CLDN18.2. Any of the bispecific T-cell engaging molecules comprising an anti-CLDN18.2 binding domain described herein can be administered to such a patient according to the methods of the invention. In certain embodiments, the bispecific T-cell engaging molecule administered according to the methods of the invention to a patient diagnosed with or having a CLDN18.2-expressing cancer (e.g. gastrointestinal cancer) is a single chain polypeptide comprising the sequence of SEQ ID NO: 160. In other embodiments, the bispecific T-cell engaging molecule administered according to the methods of the invention to a patient diagnosed with or having a CLDN18.2-expressing cancer (e.g. gastrointestinal cancer) is a single chain polypeptide comprising the sequence of SEQ ID NO: 161.

In other embodiments, the patient to be treated according to the methods of the invention is diagnosed with a MUC17-expressing cancer, such as colorectal cancer, pancreatic cancer, and gastrointestinal cancer, particularly gastric cancer and gastroesophageal junction cancer, and the anti-cancer cell antigen binding domain of the bispecific T-cell engaging molecule specifically binds to MUC17. Any of the bispecific T-cell engaging molecules comprising an anti-MUC17 binding domain described herein can be administered to such a patient according to the methods of the invention. In certain embodiments, the bispecific T-cell engaging molecule administered according to the methods of the invention to a patient diagnosed with or having a MUC17-expressing cancer (e.g. gastrointestinal cancer) is a single chain polypeptide comprising the sequence of SEQ ID NO: 171.

The bispecific T-cell engaging molecules for use in the methods of the invention may be prepared by any of a number of conventional techniques. For example, the bispecific T-cell engaging molecules described herein may be produced by recombinant expression systems, using any technique known in the art. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988).

Bispecific T-cell engaging molecules or components thereof (e.g. Fv fragments, Fc monomers) can be expressed in hybridoma cell lines or in cell lines other than hybridomas. Expression vectors or constructs encoding the bispecific T-cell engaging molecules can be used to transform a mammalian, insect or microbial host cell. The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant nucleic acid molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

Recombinant expression vectors or constructs will typically comprise a nucleic acid molecule encoding a polypeptide comprising one or more of the following: one or more CDRs provided herein; a light chain constant region; a light chain variable region; a heavy chain constant region (e.g., CH1, CH2 and/or CH3); a heavy chain variable region; hinge region, Fc domain, and/or another scaffold portion of an antibody specifically binding to a cancer cell antigen or anti-CD3 antibody. These nucleic acid sequences are inserted into an appropriate expression vector using standard ligation techniques. In embodiments in which the bispecific T-cell engaging molecule is a single chain polypeptide or single chain fusion protein, the nucleic acid comprised in the recombinant expression vector will typically encode the full-length single chain polypeptide (e.g. full-length single chain fusion protein). The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964, which is hereby incorporated by reference). Suitable expression vectors can be purchased, for example, from Invitrogen Life Technologies or BD Biosciences (formerly “Clontech”). Other useful vectors for cloning and expressing the antibody constructs and fragments include those described in Bianchi and McGrew, 2003, Biotech. Biotechnol. Bioeng. 84:439-44, which is hereby incorporated by reference. Additional suitable expression vectors are discussed, for example, in Methods Enzymol., vol. 185 (D. V. Goeddel, ed.), 1990, New York: Academic Press.

Typically, expression vectors used in any of the host cells to produce a bispecific T-cell engaging molecule will contain sequences for cloning and expression of exogenous nucleotide sequences encoding the bispecific T-cell engaging molecule or components thereof. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector may contain a “tag”-encoding sequence, i.e., an oligonucleotide molecule located at the 5′ or 3′ end of the bispecific T-cell engaging molecule coding sequence; the oligonucleotide sequence encodes polyHis (such as hexaHis), or another “tag” such as FLAG® tag, HA (hemaglutinin influenza virus), or myc, for which commercially available antibodies exist. This tag is typically fused to the polypeptide upon expression of the polypeptide and can serve as a means for affinity purification or detection of the bispecific T-cell engaging molecule from the host cell. Affinity purification can be accomplished, for example, by column chromatography using antibodies against the tag as an affinity matrix. Optionally, the tag can subsequently be removed from the purified T-cell engaging molecule by various means such as using certain peptidases for cleavage.

Expression and cloning vectors will typically contain a promoter that is recognized by the host cell and operably linked to the nucleic acid molecule encoding a bispecific T-cell engaging molecule. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system. A large number of promoters, recognized by a variety of potential host cells, are well known to those of skill in the art. For example, suitable promoters for use with mammalian host cells include those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). A suitable promoter is operably linked to the polynucleotide encoding e.g., a bispecific T-cell engaging molecule or component thereof, by removing the promoter from the source nucleic acid by restriction enzyme digestion and inserting the desired promoter sequence into the vector.

The expression vectors for recombinant production of the bispecific T-cell engaging molecules described herein may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art. The expression vectors can be introduced into host cells to thereby produce the bispecific T-cell engaging molecules encoded by the nucleic acids present in the vectors.

After the vector has been constructed and one or more nucleic acid molecules encoding the bispecific T-cell engaging molecule or component thereof has been inserted into the proper site(s) of the vector or vectors, the completed vector(s) may be inserted into a suitable host cell for amplification and/or polypeptide expression. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated polynucleotide or nucleic acid encoding a bispecific T-cell engaging molecule, preferably operably linked to at least one expression control sequence (e.g. promoter or enhancer), is a “recombinant host cell.”

The transformation of an expression vector for a polypeptide into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

A host cell, when cultured under appropriate conditions, synthesizes a bispecific T-cell engaging molecule that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. Suitable host cells include, but are not limited to, prokaryotic cells (e.g. E. coli, B. subtilis), yeast cells (Saccharmoyces cerevisiae, Pichia pastoris), and mammalian cells (e.g. Chinese hamster ovary (CHO), human embryonic kidney (HEK)). CHO cells are preferred host cells in some embodiments for expressing the bispecific T-cell engaging molecules.

Host cells are transformed or transfected with the above-described expression vectors for production of the T-cell engaging molecules and are cultured in conventional nutrient media modified as appropriate for inducing promoters, selecting transformants, or amplifying the genes encoding the desired sequences. The host cells used to produce the antibody constructs may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinary skilled artisan.

Upon culturing the host cells, the T-cell engaging molecule can be produced intracellularly, in the periplasmic space, or directly secreted into the medium. If the T-cell engaging molecule is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the T-cell engaging molecule is secreted into the culture medium, the T-cell engaging molecule can be separated from host cells through centrifugation or microfiltration, and optionally, subsequently concentrated through ultrafiltration. The bispecific T-cell engaging molecules can be further purified or partially purified using, for example, one or more chromatography steps, such as affinity chromatography (e.g. protein A, protein L, or protein G affinity chromatography), cation exchange chromatography, anion exchange chromatography, hydroxyapatite chromatography, hydrophobic interaction chromatography, or mixed mode chromatography.

Administration of the bispecific T-cell engaging molecules according to the methods of the invention is for the treatment of cancer in a patient in need thereof. The term “treatment” or “treat” as used herein refers to the application or administration of the bispecific T-cell engaging molecule to a patient who has or is diagnosed with cancer, has a symptom of cancer, is at risk of developing cancer, or has a predisposition to cancer for the purpose of curing, healing, alleviating, relieving, altering, ameliorating, or improving the cancer, one or more symptoms of the cancer, the risk of developing the cancer, or predisposition toward the cancer. The term “treatment” encompasses any improvement of the disease in the patient, including the slowing or stopping of the progression of cancer in the patient, a decrease in the number or severity of the symptoms of cancer, or an increase in frequency or duration of periods where the patient is free from the symptoms of cancer. The term “patient” includes human patients.

The term “cancer” refers to various conditions caused by the abnormal, uncontrolled growth of cells and includes neoplasms, primary tumors, secondary tumors and other metastatic lesions. Cancer can be detected in a number of ways including, but not limited to, the presence of a tumor in a tissue as detected by clinical or radiological means, detection of cancerous or abnormal cells in a biological sample (e.g. tissue biopsy), detection of a biomarker indicative of a cancer or a pre-cancerous condition, or detection of a genotype indicative of cancer or the risk of developing cancer. The term “cancer” encompasses various cancerous conditions regardless of stage, grade, invasiveness, aggressiveness, or tissue type. Cancers that may be treated according to the methods of the invention include, but are not limited to, leukemia (e.g. myeloid leukemia, chronic lymphocytic leukemia, chronic myelogenous leukemia, acute lymphoblastic leukemia), lymphoma (e.g. diffuse large B-cell lymphoma, Burkitt lymphoma, Non-Hodgkin lymphoma, follicular lymphoma), multiple myeloma, lung cancer (e.g. small-cell lung cancer (SCLC), non-small cell lung cancer (NSCLC)), glioma, glioblastoma, melanoma, prostate cancer (e.g. castration-resistant prostate cancer, neuroendocrine prostate cancer), pancreatic cancer, breast cancer, bone cancer, cervical cancer, colon cancer, colorectal cancer, endometrial cancer, head and neck cancer, liver cancer, ovarian cancer, gastric cancer, gastroesophageal junction cancer, testicular cancer, thyroid cancer, adrenal cancer, renal cancer, bladder cancer, uterine cancer, esophageal cancer, urothelial cancer, carcinoma, and sarcoma, and metastatic cancer derived from any of the foregoing.

In certain embodiments, the bispecific T-cell engaging molecule specifically binds to PSMA and CD3 and is administered according to the methods of the invention to a patient having or diagnosed with a PSMA-expressing cancer, such as prostate cancer, non-small cell lung cancer, small cell lung cancer, renal cell carcinoma, hepatocellular carcinoma, bladder cancer, testicular cancer, colon cancer, glioblastoma, breast cancer, ovarian cancer, endometrial cancer, and melanoma. In some embodiments, the PSMA-expressing cancer is prostate cancer. The prostate cancer may be castration-resistant prostate cancer (prostate cancer that is resistant to androgen deprivation therapy). In these and other embodiments, the prostate cancer is metastatic prostate cancer, particularly metastatic castration-resistant prostate cancer.

In embodiments in which a PSMA×CD3 bispecific T-cell engaging molecule (e.g. a single chain polypeptide comprising the sequence of SEQ ID NO: 60) is administered to patient in need of treatment for prostate cancer or other PSMA-expressing cancer, the methods comprise administering to the patient an initiation cycle comprising: administering a priming dose of about g to about 300 μg of the PSMA×CD3 bispecific T-cell engaging molecule by continuous intravenous infusion over a period of about 2 days or about 3 days; and administering a therapeutic dose of about 90 μg to about 1800 μg of the PSMA×CD3 bispecific T-cell engaging molecule by a bolus intravenous infusion, wherein the therapeutic dose is administered about 5 days or about 6 days after administration of the priming dose. In some embodiments, the methods comprise administering to the patient an initiation cycle comprising: administering a priming dose of about 30 μg to about 150 μg of the PSMA×CD3 bispecific T-cell engaging molecule by continuous intravenous infusion over a period of about 3 days; and administering a therapeutic dose of about 300 μg to about 600 μg of the PSMA×CD3 bispecific T-cell engaging molecule by a bolus intravenous infusion, wherein the therapeutic dose is administered about 5 days after administration of the priming dose. In other embodiments, the methods comprise administering to the patient an initiation cycle comprising: administering a priming dose of about 50 μg to about 250 μg of the PSMA×CD3 bispecific T-cell engaging molecule by continuous intravenous infusion over a period of about 5 days; and administering a therapeutic dose of about 300 μg to about 900 μg of the PSMA×CD3 bispecific T-cell engaging molecule by a bolus intravenous infusion, wherein the therapeutic dose is administered about 3 days after administration of the priming dose. In any of the foregoing embodiments, the methods may further comprise administering to the patient a maintenance cycle of the PSMA×CD3 bispecific T-cell engaging molecule, wherein the maintenance cycle comprises administering the therapeutic dose of the PSMA×CD3 bispecific T-cell engaging molecule by a bolus intravenous infusion once every 14 days.

In one particular embodiment, the method comprises administering to the patient in need of treatment for prostate cancer or other PSMA-expressing cancer an initiation cycle comprising: administering a priming dose of about 90 μg of the PSMA×CD3 bispecific T-cell engaging molecule by continuous intravenous infusion over a period of about 3 days (e.g. 30 μg per day for 3 days); and administering a therapeutic dose of about 300 μg of the PSMA×CD3 bispecific T-cell engaging molecule by a bolus intravenous infusion, wherein the therapeutic dose is administered about 5 days after administration of the priming dose. In some embodiments, the therapeutic dose (e.g. 300 μg) is subsequently administered once every 14 days for the duration of the initiation cycle. Thus, according to this dosage regimen, for an initiation cycle having a duration of 28 days, a patient would be administered the 90 μg priming dose of the PSMA×CD3 bispecific T-cell engaging molecule by continuous intravenous infusion over days 1 to 3 of the cycle (e.g. at a constant rate of 30 μg per day for 3 days) and administered the 300 μg therapeutic dose of the PSMA×CD3 bispecific T-cell engaging molecule by a bolus intravenous infusion on days 8 and 22 of the cycle.

In another particular embodiment, the method comprises administering to the patient in need of treatment for prostate cancer or other PSMA-expressing cancer an initiation cycle comprising: administering a priming dose of about 150 μg of the PSMA×CD3 bispecific T-cell engaging molecule by continuous intravenous infusion over a period of about 3 days (e.g. 50 μg per day for 3 days); and administering a therapeutic dose of about 300 μg of the PSMA×CD3 bispecific T-cell engaging molecule by a bolus intravenous infusion, wherein the therapeutic dose is administered about 5 days after administration of the priming dose. In such embodiments, the therapeutic dose (e.g. 300 μg) may be subsequently administered once every 14 days for the duration of the initiation cycle. Thus, according to this dosage regimen, for an initiation cycle having a duration of 28 days, a patient would be administered the 150 μg priming dose of the PSMA×CD3 bispecific T-cell engaging molecule by continuous intravenous infusion over days 1 to 3 of the cycle (e.g. at a constant rate of 50 μg per day for 3 days) and administered the 300 μg therapeutic dose of the PSMA×CD3 bispecific T-cell engaging molecule by a bolus intravenous infusion on days 8 and 22 of the cycle.

In another embodiment, the method comprises administering to the patient in need of treatment for prostate cancer or other PSMA-expressing cancer an initiation cycle comprising: administering a priming dose of about 150 μg of the PSMA×CD3 bispecific T-cell engaging molecule by continuous intravenous infusion over a period of about 5 days (e.g. 30 μg per day for 5 days); and administering a therapeutic dose of about 300 μg of the PSMA×CD3 bispecific T-cell engaging molecule by a bolus intravenous infusion, wherein the therapeutic dose is administered about 3 days after administration of the priming dose. In such embodiments, the therapeutic dose (e.g. 300 μg) may be subsequently administered once every 14 days for the duration of the initiation cycle. Thus, according to this dosage regimen, for an initiation cycle having a duration of 28 days, a patient would be administered the 150 μg priming dose of the PSMA×CD3 bispecific T-cell engaging molecule by continuous intravenous infusion over days 1 to 5 of the cycle (e.g. at a constant rate of 30 μg per day for 5 days) and administered the 300 μg therapeutic dose of the PSMA×CD3 bispecific T-cell engaging molecule by a bolus intravenous infusion on days 8 and 22 of the cycle.

In any of the foregoing embodiments in which a PSMA×CD3 bispecific T-cell engaging molecule is administered to a patient, the methods may further comprise administering a maintenance cycle comprising administering the therapeutic dose (e.g. 300 μg) of the PSMA×CD3 bispecific T-cell engaging molecule by a bolus intravenous infusion once every 14 days, for example on days 1 and 15 of the maintenance cycle. Depending on the duration of the initiation cycle, there may be a treatment-free period between the completion of the initiation cycle and the start of the maintenance cycle to maintain the biweekly dosing frequency of the therapeutic dose once the therapeutic dose is reached in the initiation cycle. One such exemplary dosing schedule may comprise administration of the priming dose (e.g. 90 μg or 150 μg) of the PSMA×CD3 bispecific T-cell engaging molecule by continuous intravenous infusion over days 1 to 3 and administration of the therapeutic dose (e.g. 300 μg) by a bolus intravenous infusion on days 8 and 22 of a 28-day initiation cycle, followed by a treatment-free period of 7 days, followed by administration of the therapeutic dose (e.g. 300 μg) of the PSMA×CD3 bispecific T-cell engaging molecule by a bolus intravenous infusion on day 1 and day 15 of a 28-day maintenance cycle. Thus, according to this dosing regimen, for the 56-day period encompassing both the 28-day initiation cycle and the 28-day maintenance cycle and starting with the first dose of the initiation cycle, the patient would be administered the PSMA×CD3 bispecific T-cell engaging molecule on each of days 1 to 3, day 8, day 22, day 36, and day 50. Another exemplary dosing schedule may comprise administration of the priming dose (e.g. 150 μg) of the PSMA×CD3 bispecific T-cell engaging molecule by continuous intravenous infusion over days 1 to 5 and administration of the therapeutic dose (e.g. 300 μg) by a bolus intravenous infusion on days 8 and 22 of a 28-day initiation cycle, followed by a treatment-free period of 7 days, followed by administration of the therapeutic dose (e.g. 300 μg) of the PSMA×CD3 bispecific T-cell engaging molecule by a bolus intravenous infusion on day 1 and day 15 of a 28-day maintenance cycle. Thus, according to this dosing regimen, for the 56-day period encompassing both the 28-day initiation cycle and the 28-day maintenance cycle and starting with the first dose of the initiation cycle, the patient would be administered the PSMA×CD3 bispecific T-cell engaging molecule on each of days 1 to 5, day 8, day 22, day 36, and day 50.

In certain embodiments, the bispecific T-cell engaging molecule specifically binds to BCMA and CD3 and is administered according to the methods of the invention to a patient having or diagnosed with a BCMA-positive cancer, such as multiple myeloma, heavy chain multiple myeloma, light chain multiple myeloma, extramedullary myeloma (extramedullary plasmacytoma, extramedullary multiple myeloma), plasmacytoma, plasma cell leukemia, Waldenström's macroglobulinemia (lymphoplasmacytic lymphoma), and smoldering myeloma (smoldering multiple myeloma). In some embodiments, the BCMA-positive cancer is multiple myeloma. The multiple myeloma may be refractory and/or relapsed multiple myeloma.

In some embodiments in which a BCMA×CD3 bispecific T-cell engaging molecule (e.g. a single chain polypeptide comprising the sequence of SEQ ID NO: 50) is administered to patient in need of treatment for multiple myeloma or other BCMA-positive cancer, the methods comprise administering to the patient an initiation cycle comprising: administering a priming dose of about 8,400 μg to about 16,100 μg of the BCMA×CD3 bispecific T-cell engaging molecule by continuous intravenous infusion over a period of about 7 days; and administering a therapeutic dose of about 12,000 μg to about 19,500 μg of the BCMA×CD3 bispecific T-cell engaging molecule by a bolus intravenous infusion, wherein the therapeutic dose is administered about 1 day (e.g. next day) after administration of the priming dose. The priming doses of about 8,400 μg to about 16,100 μg are total doses to be administered by the completion of the infusion period and can be translated into 7 individual doses of, e.g., from about 1,200 μg/day to about 2,300 μg/day administered on each of days 1 to 7 of the initiation cycle. In other embodiments, the methods comprise administering to the patient an initiation cycle comprising: administering a priming dose of about 4,600 μg to about 9,200 μg of the BCMA×CD3 bispecific T-cell engaging molecule by continuous intravenous infusion over a period of about 2 days; and administering a therapeutic dose of about 12,000 μg to about 19,500 μg of the BCMA×CD3 bispecific T-cell engaging molecule by a bolus intravenous infusion, wherein the therapeutic dose is administered about 6 days after administration of the priming dose. The priming doses of about 4,600 μg to about 9,200 μg are total doses to be administered by the completion of the infusion period and can be translated into 2 individual doses of, e.g., from about 2,300 μg/day to about 4,600 μg/day administered on each of days 1 and 2 of the initiation cycle. In some such embodiments, the initiation cycle may further comprise administering a boost dose of about 800 ag to about 1,600 ag of the BCMA×CD3 bispecific T-cell engaging molecule by a bolus intravenous infusion about one day (e.g. next day) after the priming dose and about five days before the therapeutic dose. In any of the foregoing embodiments, the methods may further comprise administering to the patient a maintenance cycle of the BCMA×CD3 bispecific T-cell engaging molecule, wherein the maintenance cycle comprises administering the therapeutic dose of the BCMA×CD3 bispecific T-cell engaging molecule by a bolus intravenous infusion once every 7 days.

In one particular embodiment, the method comprises administering to the patient in need of treatment for multiple myeloma or other BCMA-positive cancer an initiation cycle comprising: administering a priming dose of about 8,400 μg of the BCMA×CD3 bispecific T-cell engaging molecule by continuous intravenous infusion over a period of about 7 days (e.g. 1,200 μg per day for 7 days); and administering a therapeutic dose of about 12,000 μg to about 19,500 μg of the BCMA×CD3 bispecific T-cell engaging molecule by a bolus intravenous infusion, wherein the therapeutic dose is administered about 1 day (e.g. the next day) after administration of the priming dose. In another embodiment, the method comprises administering an initiation cycle comprising: administering a priming dose of about 16,100 μg of the BCMA×CD3 bispecific T-cell engaging molecule by continuous intravenous infusion over a period of about 7 days (e.g. 2,300 μg per day for 7 days); and administering a therapeutic dose of about 12,000 μg to about 19,500 μg of the BCMA×CD3 bispecific T-cell engaging molecule by a bolus intravenous infusion, wherein the therapeutic dose is administered about 1 day (e.g. the next day) after administration of the priming dose. In any of the foregoing embodiments, the therapeutic dose may be subsequently administered once every 7 days for the duration of the initiation cycle. Thus, according to such dosage regimens, for an initiation cycle having a duration of 28 days, a patient would be administered the priming dose (e.g. 8,400 μg or 16,100 μg) of the BCMA×CD3 bispecific T-cell engaging molecule by continuous intravenous infusion over days 1 to 7 of the cycle (e.g. at a constant rate of 1,200 μg per day for 7 days for the 8,400 μg priming dose or 2,300 μg per day for 7 days for the 16,100 μg priming dose) and administered the therapeutic dose of the BCMA×CD3 bispecific T-cell engaging molecule by a bolus intravenous infusion on days 8, 15, and 22 of the cycle.

In another particular embodiment, the method comprises administering to the patient in need of treatment for multiple myeloma or other BCMA-positive cancer an initiation cycle comprising: administering a priming dose of about 4,600 μg of the BCMA×CD3 bispecific T-cell engaging molecule by continuous intravenous infusion over a period of about 2 days (e.g. 2,300 μg per day for 2 days); administering a boost dose of about 800 μg of the BCMA×CD3 bispecific T-cell engaging molecule by a bolus intravenous infusion, and administering a therapeutic dose of about 12,000 μg to about 19,500 μg of the BCMA×CD3 bispecific T-cell engaging molecule by a bolus intravenous infusion, wherein the therapeutic dose is administered about 6 days after administration of the priming dose and the boost dose is administered about 1 day (e.g. next day) after the priming dose and about 5 days before the therapeutic dose. In another embodiment, the method comprises administering an initiation cycle comprising: administering a priming dose of about 9,200 μg of the BCMA×CD3 bispecific T-cell engaging molecule by continuous intravenous infusion over a period of about 2 days (e.g. 4,600 μg per day for 2 days); administering a boost dose of about 1,600 μg of the BCMA×CD3 bispecific T-cell engaging molecule by a bolus intravenous infusion, and administering a therapeutic dose of about 12,000 μg to about 19,500 μg of the BCMA×CD3 bispecific T-cell engaging molecule by a bolus intravenous infusion, wherein the therapeutic dose is administered about 6 days after administration of the priming dose and the boost dose is administered about 1 day (e.g. next day) after the priming dose and about 5 days before the therapeutic dose. In any of the foregoing embodiments, the therapeutic dose may be subsequently administered once every 7 days for the duration of the initiation cycle. Thus, according to the dosage regimens in these embodiments, for an initiation cycle having a duration of 28 days, a patient would be administered the priming dose (e.g. 4,600 μg or 9,200 μg) of the BCMA×CD3 bispecific T-cell engaging molecule by continuous intravenous infusion over days 1 to 2 of the cycle (e.g. at a constant rate of 2,300 μg per day for 2 days for the 4,600 μg priming dose or 4,600 μg per day for 2 days for the 9,200 μg priming dose), administered a boost dose (e.g. 800 μg or 1,600 μg) of the BCMA×CD3 bispecific T-cell engaging molecule by a bolus intravenous infusion on day 3 of the cycle, and administered the therapeutic dose of the BCMA×CD3 bispecific T-cell engaging molecule by a bolus intravenous infusion on days 8, 15, and 22 of the cycle.

In any of the foregoing embodiments in which a BCMA×CD3 bispecific T-cell engaging molecule is administered to a patient, the methods may further comprise administering a maintenance cycle comprising administering the therapeutic dose of the BCMA×CD3 bispecific T-cell engaging molecule by a bolus intravenous infusion once every 7 days, for example on days 1, 8, 15, and 22 of the maintenance cycle. Depending on the duration of the initiation cycle, there may be no treatment-free period between the completion of the initiation cycle and the start of the maintenance cycle in order to maintain the weekly dosing frequency of the therapeutic dose once the therapeutic dose is reached in the initiation cycle. Accordingly, in certain embodiments, the maintenance cycle is administered the following day after completing the initiation cycle. One such exemplary dosing schedule may comprise administration of the priming dose of the BCMA×CD3 bispecific T-cell engaging molecule by continuous intravenous infusion over days 1 to 7 and administration of the therapeutic dose by a bolus intravenous infusion on days 8, 15, and 22 of a 28-day initiation cycle, followed by administration of the therapeutic dose of the BCMA×CD3 bispecific T-cell engaging molecule by a bolus intravenous infusion on days 1, 8, 15, and 22 of a 28-day maintenance cycle. Thus, according to this dosing regimen, for the 56-day period encompassing both the 28-day initiation cycle and the 28-day maintenance cycle and starting with the first dose of the initiation cycle, the patient would be administered the BCMA×CD3 bispecific T-cell engaging molecule on each of days 1 to 7, day 8, day, 15, day 22, day 29, day 36, day 43, and day 50.

In certain embodiments of the methods of the invention, one or more premedications can be administered to the patient prior to the administration of a first dose of a bispecific T-cell engaging molecule in the initiation cycle. In some embodiments, the premedication is administered to the patient prior to administration of each dose of the bispecific T-cell engaging molecule in the initiation cycle. The premedication may also be administered to the patient prior to administration of one or more doses of the bispecific T-cell engaging molecule in one or more maintenance cycles. In some embodiments, the premedication is only administered to the patient prior to administration of one or more doses during the initiation cycle and is not administered to the patient prior to administration of any dose of the bispecific T-cell engaging molecule in a subsequent treatment cycle (e.g. a maintenance cycle). In alternative embodiments, the premedication is administered to the patient prior to administration of one or more doses during the initiation cycle but is administered to the patient at a lower dose (e.g. 50% of the premedication dose employed in the initiation cycle) prior to administration of a dose of the bispecific T-cell engaging molecule in a subsequent treatment cycle (e.g. a maintenance cycle). It is envisaged that “prior to”, in this specific context, means within 72 hours, 48 hours, 36, hours, 24 hours, 18 hours, 16 hours, 12 hours, 6 hours, 5 hours, 4 hours, or 3 hours, and preferably within 120, 90, 60 or 30 minutes before the start of administration of the bispecific T-cell engaging molecule. Depending on the type of premedication used and the route by which it is administered, the premedication may e.g. be administered 30-120 or 30-60 minutes prior to start of administration of the bispecific T-cell engaging molecule. The premedication may be administered e.g. to prevent or reduce severity of infusion-related reactions and/or to prevent or reduce severity of cytokine release syndrome or its symptoms. In certain embodiments, no premedication is administered prior to any dose of the bispecific T-cell engaging molecule in the initiation cycle or is administered at lower doses than is typically necessary to reduce infusion reactions or CRS symptoms. Without being bound by theory, it is believed that administration of the first dose of the bispecific T-cell engaging molecule in the initiation cycle by a continuous infusion according to the dosing regimens described herein reduces CRS events such that premedication may no longer be necessary.

In some embodiments in which premedication is administered, the premedication is an antihistamine. The antihistamine can be administered orally or intravenously and can be administered at a dose equivalent to diphenhydramine 50 mg i.v. Suitable antihistamines that can be administered as a premedication include, but are not limited to, antihistamines of oral, parenteral or rectal route such as: azatadine (maximum dose e.g. 4 mg/day), brompheniramine (maximum dose e.g. 30 mg/day), cetirizine (maximum dose e.g. 15 mg/day), chlorpheniramine (maximum dose e.g. 30 mg/day), clemastine (maximum dose e.g. 10 mg/day), cyproheptadine (maximum dose e.g. 15 mg/day), desloratadine (maximum dose e.g. 7 mg/day), dexchlorpheniramine (maximum dose e.g. 15 mg/day), diphenhydramine (maximum dose e.g. 350 mg/per day), doxylamine (maximum dose e.g. 180 mg/day), fexofenadine (maximum dose e.g. 200 mg/day), loratadine (maximum dose e.g. 15 mg/day), and phenindamine (maximum dose e.g. 180 mg/day).

In other embodiments in which premedication is administered, the premedication is a glucocorticoid. Glucocorticoids are a class of corticosteroids, which are a class of steroid hormones. Glucocorticoids are corticosteroids that bind to the glucocorticoid receptor. A less common synonym is glucocorticosteroid. Cortisol (known as hydrocortisone when used as a medication) is the most important human glucocorticoid. A variety of synthetic glucocorticoids, some far more potent than cortisol, have been created for therapeutic use. Cortisol is the standard of comparison for glucocorticoid potency. One example for commonly prescribed replacement steroid equivalents may be prednisone (5 mg)=cortisone (25 mg)=dexamethasone (0.75 mg)=hydrocortisone (20 mg)=methylprednisolone (4 mg). These doses indicate the equivalent pharmacologic dose of systemic glucocorticoids. The glucocorticoid can be administered orally or intravenously and can be administered at a dose equivalent to 4-20 mg dexamethasone i.v. (the equivalence referring to the glucocorticoid potency). The dose of glucocorticoid can be the same at each administration (i.e. at each time the glucocorticoid premedication is administered). Alternatively, the dose of glucocorticoid can be reduced in subsequent administrations, e.g. by 50% of the previous dose, if there are no or minimal signs of infusion reactions and/or CRS symptoms following the previous administration of the bispecific T-cell engaging molecule. In certain embodiments, glucocorticoids are only administered as premedications during the initiation cycle and are not administered in subsequent treatment cycles (e.g. maintenance cycles).

Examples of glucocorticoids to be used as a premedication include, but are not limited to, cortisone, hydrocortisone, prednisone, prednisolone, methylprednisolone, dexamethasone, betamethasone, beclomethasone, budesonide, triamcinolone, cloprednol, deflazacort, fluocortolone, cortivazol, paramethasone, fluticasone, fluticasone propionate, triamcinolone acetonide, as well as combinations and/or pharmaceutically acceptable derivatives thereof. The different glucocorticoids may be used alone or in combination. Dexamethasone, prednisone and prednisolone are preferred glucocorticoids for use as a premedication according to the methods of the invention. In certain embodiments of the methods of the invention, the glucocorticoid administered to the patient prior to administration of one or more (or all) doses of the bispecific T-cell engaging molecule during the initiation cycle and/or maintenance cycle is dexamethasone. Dexamethasone can be administered at a dose of about 4-20 mg, 6-18 mg, 8-16 mg, about 16 mg, or about 8 mg at each administration.

In certain embodiments in which a premedication is administered, the premedication can be an IL-6 receptor antagonist, such as tocilizumab. Tocilizumab has been reported to effectively reduce or reverse symptoms of CRS induced by T cell-engaging therapies. See, e.g., Maude et al., Cancer J., Vol. 20:119-122, 2014. Tocilizumab can be administered at a dose of about 1 mg/kg to about 20 mg/kg body weight, about 8 mg/kg to about 12 mg/kg body weight, or about 4 mg/kg to about 8 mg/kg body weight. Tocilizumab can be administered about 1 hour to about 2 hours prior to each dose of the bispecific T-cell engaging molecule in the initiation cycle and/or one or more maintenance cycles. Additionally or alternatively, tocilizumab can be administered immediately after each dose of the bispecific T-cell engaging molecule in the initiation cycle and/or one or more maintenance cycles. Other antagonists of IL-6/IL-6 receptor signaling, such as siltuximab, olokizumab, clazakizumab, sarilumab, and sirukumab, can be used as a premedication according to the methods of the invention to reduce the occurrence or severity of CRS.

In certain other embodiments in which a premedication is administered, the premedication is a tumor necrosis factor alpha (TNF-alpha) antagonist. CRS symptoms have been previously reported to be mediated in part by release of TNF-alpha (Lee et al., Blood, Vol. 124:188-195, 2014; Grupp et al., N Engl J Med., Vol. 368:1509-1518, 2013). Recent studies have suggested that treatment with TNF-alpha antagonists prior to administration of immunotherapy agents may mitigate CRS symptoms (Li et al., Sci Transl Med., Vol. 11(508), 2019; Lee et al., 2014, supra; Grupp et al., 2013, supra). Accordingly, in certain embodiments, the methods of the invention further comprise administering to the patient a TNF-alpha antagonist prior to administration of each dose of the bispecific T-cell engaging molecule during the initiation cycle and/or one or more maintenance cycles. Examples of TNF-alpha antagonists that can be used as a premedication include, but are not limited to, etanercept, infliximab, adalimumab, certolizumab pegol, and golimumab. In particular embodiments of the methods of the invention, the TNF-alpha antagonist administered to the patient prior to administration of one or more (or all) doses of the bispecific T-cell engaging molecule during the initiation cycle and/or maintenance cycle is etanercept. Etanercept can be administered at a dose of about 10 mg to 100 mg, about 25 mg to about 75 mg, about 40 mg to about 60 mg, or about 50 mg at each administration and can be administered subcutaneously or intravenously. In some embodiments of the methods of the invention, etanercept is administered to the patient prior to the administration of each dose of the bispecific T-cell engaging molecule during the initiation cycle. In some such embodiments, etanercept is subcutaneously administered to the patient at a dose of about 50 mg about 2 days prior to administration of each dose of the bispecific T-cell engaging molecule during the initiation cycle. In other such embodiments, etanercept is subcutaneously administered to the patient at a dose of about 50 mg about 1 day prior to administration of each dose of the bispecific T-cell engaging molecule during the initiation cycle.

A patient may be treated according to the methods of the invention for a set treatment period. A “treatment period” begins upon administration of a first dose of a bispecific T-cell engaging molecule in an initiation cycle and ends upon administration of a final dose of a bispecific T-cell engaging molecule in a maintenance cycle. The treatment period may be from about 3 months to about 36 months, from about 12 months to about 24 months, or from about 6 months to about 12 months. For instance, the treatment period may be about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, about 33 months, or about 36 months. In some embodiments, the treatment period is about 6 months. In other embodiments, the treatment period is about 9 months. In yet other embodiments, the treatment period is about 12 months. The treatment period can be adjusted for each patient depending on the patient's response to treatment. In one particular embodiment, the patient is treated according to the methods of the invention until the patient achieves a complete response or until evidence of the particular cancer is otherwise undetectable in the patient.

The bispecific T-cell engaging molecule is generally administered to the patient in a pharmaceutical composition, which can include pharmaceutically-acceptable carriers, excipients, or diluents. “Pharmaceutically-acceptable” refers to molecules, compounds, and compositions that are non-toxic to human recipients at the dosages and concentrations employed and/or do not produce allergic or adverse reactions when administered to humans. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company. Pharmaceutical compositions comprising the bispecific T-cell engaging molecules to be administered according to the methods of the invention include, but are not limited to, liquid, frozen, and lyophilized compositions.

If the pharmaceutical composition has been lyophilized, the lyophilized material is reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

In some embodiments, the selection of carriers and excipients for incorporation into the pharmaceutical compositions influences the physical state, stability, rate of in vivo release and rate of in vivo clearance of the bispecific T-cell engaging molecules. In certain embodiments, the primary vehicle or carrier in a pharmaceutical composition may be either aqueous or non-aqueous in nature. For example, a suitable vehicle or carrier may be water for injection, physiological saline solution, possibly supplemented with other materials or excipients common in compositions for parenteral administration.

In the methods described herein, the bispecific T-cell engaging molecule (e.g. a pharmaceutical composition comprising the bispecific T-cell engaging molecule) is administered to the patient parenterally. Parenteral administration refers to administration of the molecule by routes other than through the gastrointestinal tract and can include intraperitoneal, intramuscular, intravenous, intraarterial, intradermal, subcutaneous, intracerebral, intracerebroventricular, and intrathecal administration. In preferred embodiments, administration of the bispecific T-cell engaging molecule according to the methods of the invention is intravenous. In other preferred embodiments, administration of the bispecific T-cell engaging molecule according to the methods of the invention is subcutaneous. In certain embodiments of the methods of the invention, a priming dose of the bispecific T-cell engaging molecule is administered by a continuous intravenous infusion and administration of boost doses and/or therapeutic doses of the bispecific T-cell engaging molecule are administered by a bolus intravenous infusion. In certain other embodiments of the methods of the invention, a priming dose of the bispecific T-cell engaging molecule is administered by a continuous intravenous infusion and administration of boost doses and/or therapeutic doses of the bispecific T-cell engaging molecule are administered by a subcutaneous injection.

Parenteral, subcutaneous, or intravenous administration can be performed by injection (e.g. using a needle and a syringe) or by infusion (e.g. via a catheter and a pump system). It is envisaged that in some embodiments the administration according to the present invention is via intravenous injection or via intravenous infusion. Usually, an intravenous (IV) infusion is administered via a line, a port or a catheter (small, flexible tube), such as a central venous access or a central venous catheter (CVC), which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

In certain embodiments, the pharmaceutical compositions comprise an effective amount of the bispecific T-cell engaging molecule and one or more excipients. An effective amount can be a therapeutic dose, or it may be a smaller amount, such as a priming dose or boost dose. Excipients can be used for a wide variety of purposes, such as adjusting physical, chemical, or biological properties of formulations, such as adjustment of viscosity, and/or to stabilize such formulations against degradation and spoilage e.g. due to stresses that occur during manufacturing, shipping, storage, pre-use preparation, and administration.

In some embodiments, the pharmaceutical composition comprising an effective amount of a bispecific T-cell engaging molecule to be administered to a patient according to the methods of the invention comprises a buffer. Buffers are used to maintain the composition at physiological pH or at a slightly lower pH, typically within a pH range from about 4.0 to about 6.5. Suitable buffers include, but are not limited to, glutamate, aspartate, acetate, Tris, citrate, histidine, succinate, and phosphate buffers. In certain embodiments, the pharmaceutical composition administered according to the methods described herein comprises a glutamate buffer, particularly L-glutamate buffer. Pharmaceutical compositions comprising a glutamate buffer can have a pH of about 4.0 to about 5.5, a pH of about 4.0 to about 4.4, or a pH of about 4.2 to about 4.8.

The pharmaceutical composition comprising an effective amount of a bispecific T-cell engaging molecule may further comprise a surfactant. The term “surfactant” as used herein refers to a substance that functions to reduce the surface tension of a liquid in which it is dissolved. Surfactants can be included in pharmaceutical compositions for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants that may be incorporated into the pharmaceutical compositions used in the methods of the invention include both non-ionic and ionic surfactants. Suitable non-ionic surfactants include, but are not limited to, alkyl poly (ethylene oxide), alkyl polyglucosides, such as octyl glucoside and decyl maltoside, fatty alcohols, such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Suitable ionic surfactants include, for example, anionic, cationic and zwitterionic surfactants. Anionic surfactants include, but are not limited to, sulfonate-based or carboxylate-based surfactants such as soaps, fatty acid salts, sodium dodecyl sulfate (SDS), ammonium lauryl sulfate and other alkyl sulfate salts. Cationic surfactants include, but are not limited to, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate. In certain embodiments, the pharmaceutical compositions administered according to the methods described herein comprise a non-ionic surfactant. In one embodiment, the non-ionic surfactant is polysorbate 20. In another embodiment, the non-ionic surfactant is polysorbate 80.

In certain embodiments, the pharmaceutical composition comprising an effective amount of a bispecific T-cell engaging molecule further comprises a stabilizing agent. As used herein, the term “stabilizing agent” refers to an excipient that stabilizes the native conformation of the polypeptide or T-cell engaging molecule and/or prevents or reduces the physical or chemical degradation of the polypeptide or T-cell engaging molecule. Suitable stabilizing agents include, but are not limited to, polyols (e.g. sorbitol, glycerol, mannitol, xylitol, maltitol, lactitol, erythritol and threitol), sugars (e.g., fructose, glucose, glyceraldehyde, lactose, arabinose, mannose, xylose, ribose, rhamnose, galactose maltose, sucrose, trehalose, sorbose, sucralose, melezitose and raffinose), and amino acids (e.g., glycine, methionine, proline, lysine, arginine, histidine, or glutamic acid). In some embodiments, the pharmaceutical composition comprises a sugar as a stabilizing agent. In these and other embodiments, the sugar is sucrose.

Exemplary pharmaceutical compositions comprising bispecific T-cell engaging molecules are described in WO 2018/141910, which is hereby incorporated by reference in its entirety. In certain embodiments, a pharmaceutical composition useful for the treatment of cancer according to the methods described herein comprises about 0.5 mg/ml to about 2 mg/ml of a bispecific T-cell engaging molecule, about 5 mM to about 20 mM L-glutamic acid, about 0.005% to about 0.015% weight/volume (w/v) polysorbate (e.g. polysorbate 20 or polysorbate 80), and about 7% to about 12% (w/v) sucrose. In other embodiments, the pharmaceutical composition comprises about 0.5 mg/ml to about 1.5 mg/ml of a bispecific T-cell engaging molecule, about 8 mM to about 12 mM L-glutamic acid, about 0.008% to about 0.012% (w/v) polysorbate (e.g. polysorbate 20 or polysorbate 80), and about 8% to about 10% (w/v) sucrose. The pH of these compositions is in the range of about 4.0 to about 4.4 (e.g., pH of about 4.0, about 4.1, about 4.2, about 4.3, or about 4.4).

Any of the pharmaceutical compositions comprising the bispecific T-cell engaging molecules described herein can be lyophilized and reconstituted with, e.g. sterile water for injection, prior to administration to the patient. Reconstitution volumes will depend on the protein content following lyophilization and the desired concentration of the bispecific T-cell engaging molecule in the reconstituted solution, but may be from about 0.5 ml to about 5 ml. The solution following reconstitution can be further diluted with a diluent (e.g. saline and/or intravenous solution stabilizer (IVSS)) prior to administration to the patient as appropriate in order to administer the doses described herein according to the methods of the invention.

Any of the bispecific T-cell engaging molecules described herein can be incorporated into any of the pharmaceutical compositions described above and administered to a patient according to the methods described herein. In a preferred embodiment, the PSMA×CD3 bispecific T-cell engaging molecule administered according to the methods of the invention for the treatment of prostate cancer or other PSMA-expressing cancer comprises the amino acid sequence of SEQ ID NO: 60. In another preferred embodiment, the BCMA×CD3 bispecific T-cell engaging molecule administered according to the methods of the invention for the treatment of multiple myeloma or other BCMA-positive cancer comprises the amino acid sequence of SEQ ID NO: 50.

The present invention also includes kits for treating cancer in a patient in need thereof. In one embodiment, the kit comprises a pharmaceutical composition of a bispecific T-cell engaging molecule described herein and packaging material that provides instructions regarding the use of the pharmaceutical compositions. The pharmaceutical composition of the kit may be present in a container, such as a vial. The pharmaceutical composition may be provided as a solution, suspension, gel, emulsion, solid, crystal, or as a dehydrated or lyophilized powder. In embodiments in which the pharmaceutical composition is provided as a lyophilized powder, the kit may also comprise diluents (e.g. sterile water for injection, saline, phosphate-buffer saline, formulation buffer) necessary to reconstitute the pharmaceutical composition as well as instructions for preparing the composition for administration. In certain embodiments, the kits may further comprise one or more vials of intravenous solution stabilizer (IVSS) and instructions for using the IVSS for pre-treatment of IV bags prior to dilution of the pharmaceutical composition for delivery to the patient. IVSS does not contain an active pharmaceutical ingredient and is typically a buffered, preservative-free solution. In one embodiment, IVSS comprises citric acid (e.g. 20-30 mM), lysine hydrochloride (e.g. 1-3 M), and polysorbate 80 (0.05%-0.15% (w/v)) at pH 7.0. In a particular embodiment, IVSS comprises 25 mM citric acid, 1.25 M lysine hydrochloride, and 0.1% (w/v) polysorbate 80 at pH 7.0.

The following examples, including the experiments conducted and the results achieved, are provided for illustrative purposes only and are not to be construed as limiting the scope of the appended claims.

### EXAMPLES

### Example 1. Comparison of the Safety and Efficacy of Cycle 1 Priming Dose Regimens for a PSMA×CD3 Bispecific T-Cell Engaging Molecule

Bispecific T-cell engaging molecules are designed to direct T lymphocyte effector cells towards target cancer cells. The proximity of the T-cell to the target cancer cell induced by the bispecific T-cell engaging molecule triggers T-cell activation resulting in the T-cell-mediated cytotoxicity of the target cancer cell. T-cell activation mediated by bispecific T-cell engaging molecules not only induces the directed release of cytotoxic proteins to target cancer cells, but also results in a production of inflammatory cytokines, such as interferon gamma (IFN-γ), tumor necrosis factor (TNF), interleukin-2 (IL-2), and interleukin-6 (IL-6) by the T cells. The production of these inflammatory cytokines can lead to cytokine release syndrome (CRS), an adverse side effect associated with treatment with a bispecific T-cell engaging molecule.

AMG 160 is a half-life extended (HLE) BiTE® (bispecific T-cell engager) molecule that binds both prostate-specific membrane antigen (PSMA) and CD3 and comprises a single chain IgG Fc domain. The amino acid sequence of AMG 160 is set forth in SEQ ID NO: 60. Data from initial cohorts in the dose exploration portion of a phase 1 study of AMG 160 in adult patients with metastatic castration-resistant prostate cancer (mCRPC) showed that when AMG 160 was administered as a short-term (e.g. approximately 60 min) intravenous (IV) infusion once every two weeks (Q2W) in a 28-day cycle, the degree of CRS exhibited by the patients appeared to be correlated with peak serum levels (e.g. Cₘₐₓ) of AMG 160 as well as serum IL-6 levels measured about six hours after the administration of the first dose. As a mitigation strategy to reduce CRS during cycle 1, the cycle 1 dosing schedule in the phase 1 study was modified to either: (i) a dosing schedule including one, two, or three-step doses of AMG 160 administered at a weekly interval until the target dose was reached or (ii) a dosing schedule involving administration of the first dose by a continuous IV infusion over the course of 2 to 3 days followed by short IV infusions of the target dose every two weeks. Without being bound by theory, it is believed that administration of the first dose (i.e. priming dose) of AMG 160 by a continuous IV infusion over 2 to 3 days will decrease Cₘₐₓ and delay Tₘₐₓ of AMG 160 while maintaining cumulative exposures during the first dosing interval such that one or more of the following occurs: the frequency and severity of CRS events are decreased, T-cell-mediated cytokine release is downregulated while maintaining T-cell cytotoxic potential, and/or efficacious doses of AMG 160 are delivered as early as possible in cycle 1.

After signing informed consent, patients entered the screening period (up to 28 days), during which eligibility of the patients was assessed. Eligible patients had mCRPC refractory to prior novel hormonal therapy and 1 to 2 taxane regimens and evidence of progressive disease. Specifically, patients were enrolled in the study if they met all of the following key inclusion criteria:
histologically or cytologically confirmed mCRPC who were refractory to a novel anti-androgen therapy (e.g., abiraterone, enzalutamide, darolutamide, and/or apalutamide) and had failed at least 1 (but not more than 2) taxane regimens (or who were deemed medically unsuitable to be treated with a taxane regimen or actively refused treatment with a taxane regimen);
had undergone bilateral orchiectomy or were on continuous androgen-deprivation therapy (ADT) with a gonadotropin-releasing hormone (GnRH) agonist or antagonist;
had a total serum testosterone level ≤50 ng/dL or 1.7 nmol/L; and
had evidence of progressive disease, defined by one or more of the following Prostate Cancer Working Group 3 (PCWG3; Scher et al., J. Clin, Oncol, Vol. 34:1402-1418, 2016) criteria:
   prostate-specific antigen (PSA) level≥1 ng/mL that has increased on at least 2 successive occasions at least 1 week apart
   nodal or visceral progression as defined by Response Evaluation Criteria in Solid Tumors (RECIST) 1.1 with PCGW3 modifications
   appearance of 2 or more new lesions in bone scan

Patients were excluded from the study if they: (i) had active autoimmune disease requiring immunosuppressive therapy; (ii) received a prior PSMA-targeted therapy with the exception of a PSMA radioligand therapy, or (iii) had CNS metastases, leptomeningeal disease, or spinal cord compression.

AMG 160 was administered as a short IV infusion (approximately 60 minutes) every two weeks (Q2W)(e.g. on days 1 and 15) after target dose was reached in a 28-day cycle at target doses ranging from 0.003 to 0.9 mg. The date of the first dose of AMG 160 was defined as day 1 in the cycle. Two different cycle 1 priming dose strategies were implemented to reduce the incidence and/or severity of CRS. The first cycle 1 priming dose strategy was a step-dosing strategy and included single-step, two-step, and three-step dosing schedules in cycle 1. Single-step dosing involved a run-in dose (e.g. a priming dose) of AMG 160 administered on cycle 1 day 1 followed by administration of the target dose of AMG 160 on days 8 and 22 of a 28-day cycle (plus a 7-day infusion-free interval before start of cycle 2). A two-step dosing schedule entailed administration of a run-in dose (e.g. a first priming dose) of AMG 160 on cycle 1 day 1 followed by administration of a higher run-in dose (e.g. a second priming dose) of AMG 160 on cycle 1 day 8, and then administration of the target dose of AMG 160 on cycle 1 day 15 of a 28-day cycle. A three-step dosing schedule involved administration of a run-in dose (e.g. a first priming dose) of AMG 160 on cycle 1 day 1 followed by administration of a higher run-in dose (e.g. a second priming dose) of AMG 160 on cycle 1 day 8 followed by administration of another higher run-in dose (e.g. a third priming dose) of AMG 160 on cycle 1 day 15, and then administration of the target dose of AMG 160 on cycle 1 day 22 of a 28-day cycle (plus a 7-day infusion-free interval before start of cycle 2).

The second cycle 1 priming dose strategy (cIV priming; also referred to herein as extended IV priming or eIV priming) involved a run-in dose (e.g. priming dose) administered via a 2-day or 3-day continuous IV infusion of AMG 160 on cycle 1 days 1 to 2 or cycle 1 days 1 to 3 followed by administration of the target dose of AMG 160 by short-term IV infusion (approx. 60 min infusion) on cycle 1 day 8 and day 22 of a 28-day cycle (plus a 7-day infusion-free interval before start of cycle 2). Relative to a short-term IV infusion (e.g. 60 min infusion) of a particular priming dose, a 3-day continuous IV infusion of the same priming dose was projected to lower the peak serum exposures (Cₘₐₓ) of AMG 160 by approximately 40% and to delay the Tₘₐₓ (i.e. time to Cₘₐₓ) to reduce the incidence or severity of CRS and downregulate cytokine release by T cells. The priming dose was administered at a constant rate over the indicated period of days (e.g. over 2 or 3 days). For example, for a priming dose of 0.03 mg administered over 3 days, the priming dose was infused continuously at a constant rate to deliver 0.01 mg/day for 3 days. Similarly, for a priming dose of 0.30 mg administered over 3 days, the priming dose was infused continuously at a constant rate to deliver 0.10 mg/day for 3 days.

After cycle 1, cycle 2 and all subsequent cycles entailed the administration of the target dose as a short IV infusion (e.g. approx. 60 min) of AMG 160 on days 1 and 15 of the 28-day cycle. Table 1 below summarizes the different dosing cohorts. For cohorts that were dosed according to a no-step dosing regimen or a two-step dosing regimen, cycle 2 was initiated immediately following the 28-day cycle 1—that is, study day 29 was day 1 of cycle 2. For cohorts dosed according to a single-step or three-step dosing regimen or dosed according to a cIV priming dosing regimen, cycle 2 was initiated 7 days after the 28-day cycle 1—i.e. study day 36 was day 1 of cycle 2. All patients were pre-treated with 8 mg PO dexamethasone 6-16 hours prior to all doses of AMG 160 in cycle 1. Additionally, dexamethasone 8 mg IV was administered within 1 hour prior to all doses of AMG 160 in cycle 1. Patients received treatment cycles of AMG 160 until disease progression or unacceptable toxicities.

Anti-tumor activity of AMG 160 was evaluated by several measures, including objective response per RECIST 1.1 criteria with PCWG3 modifications, PSA response, circulating tumor cells (CTC) response, radiographic response as measured by ⁶⁸Gallium (⁶⁸Ga)-PSMA-11 positron emission tomography (PET)/computed tomography (CT) and ¹⁸F-fluorodeoxyglucose (FDG) PET/CT scans, progression-free survival (radiographic and PSA), and overall survival. CT/magnetic resonance imaging (MRI) scans were performed at baseline and every 8 weeks for the first 6 months of treatment and then every 12 weeks thereafter. Tumor burden assessments were performed based on RECIST 1.1 with PCWG3 modifications (see Eisenhauer et al., European Journal of Cancer, Vol. 45: 228-247, 2009; Scher et al., J. Clin, Oncol, Vol. 34:1402-1418, 2016). To confirm disease progression (PD), a second MRI/CT scan was performed 4-6 weeks after the first detection of radiographical progression. Responses (partial response (PR) and complete response (CR)) were confirmed by a repeat consecutive assessment at least 4 weeks after the first detection of radiographical response.

PSA30/50/70/90 responses were defined as 3000, 5000, 7000, and 90% reduction, respectively, in serum PSA levels from baseline. CTC response was defined as CTC0 (reduction of CTCs >0 to 0) or CTC conversion (≥5 CTCs/7.5 mL blood to ≤4 CTCs/7.5 mL blood) measured in whole blood. ⁶⁸Ga-PSMA-11 PET/CT scans were performed at baseline to assess PSMA-positive tumor burden and every 12 weeks during treatment for response assessment. To identify PSMA-negative disease burden, ¹⁸F-FDG PET/CT scans were performed at baseline and every 12 weeks during treatment for response assessment during the dose expansion phase.

TABLE 1
Summary of AMG 160 Dosing Regimen Cohorts

| | Study day¹ | D1 | D8 | D15 | D22 | D29 | D36 | D43 | D50 |
|---|---|---|---|---|---|---|---|---|---|
| No | Cohort 1 | 0.003 | | 0.003 | | 0.003 | | 0.003 | |
| Step- | (n = 3) | mg | | mg | | mg | | mg | |
| Dose | Cohort 2 | 0.01 | | 0.01 | | 0.01 | | 0.01 | |
| | (n = 4) | mg | | mg | | mg | | mg | |
| | Cohort 3a | 0.03 | | 0.03 | | 0.03 | | 0.03 | |
| | (n= 1) | mg | | mg | | mg | | mg | |
| Single | Cohort 3b | 0.01 | 0.03 | | 0.03 | | 0.03 | | 0.03 |
| Step- | (n = 3) | mg | mg | | mg | | mg | | mg |
| Dose | Cohort 4 | 0.01 | 0.09 | | 0.09 | | 0.09 | | 0.09 |
| | (n = 6) | mg | mg | | mg | | mg | | mg |
| Two | Cohort 5 | 0.01 | 0.09 | 0.30 | | 0.30 | | 0.30 | |
| Step- | (n = 4) | mg | mg | mg | | mg | | mg | |
| Dose | Cohort 6a | 0.01 | 0.09 | 0.90 | | 0.90 | | 0.90 | |
| | (n = 4) | mg | mg | mg | | mg | | mg | |
| | Cohort 6b | 0.03 | 0.09 | 0.90 | | 0.90 | | 0.90 | |
| | (n = 4) | mg | mg | mg | | mg | | mg | |
| Three | Cohort 6c | 0.01 | 0.03 | 0.09 | 0.90 | | 0.90 | | 0.90 |
| Step- | (n = 4) | mg | mg | mg | mg | | mg | | mg |
| Dose | | | | | | | | | |
| | | D1-3 | D8 | D15 | D22 | D29 | D36 | D43 | D50 |
| cIV | cIV cohort 1 | 0.03 | 0.09 | | 0.09 | | 0.09 | | 0.09 |
| priming | (n = 2) | mg | mg | | mg | | mg | | mg |
| | cIV cohort 2a | 0.09 | 0.30 | | 0.30 | | 0.30 | | 0.30 |
| | (n = 3) | mg | mg | | mg | | mg | | mg |
| | cIV cohort 3a | 0.30 | 0.90 | | 0.90 | | 0.90 | | 0.90 |
| | (n = 1) | mg | mg | | mg | | mg | | mg |
| | cIV cohort 4² | 0.15 | 0.60 | | 0.60 | | 0.60 | | 0.60 |
| | | mg | mg | | mg | | mg | | mg |
| | cIV cohort 5² | 0.15 | 0.30 | | 0.30 | | 0.30 | | 0.30 |
| | | mg | mg | | mg | | mg | | mg |
| | | D1-2 | D8 | D15 | D22 | D29 | D36 | D43 | D50 |
| | cIV cohort 2b | 0.09 | 0.30 | | 0.30 | | 0.30 | | 0.30 |
| | (n = 4) | mg | mg | | mg | | mg | | mg |
| | cIV cohort 3b | 0.09 | 0.90 | | 0.90 | | 0.90 | | 0.90 |
| | (n = 2) | mg | mg | | mg | | mg | | mg |

¹Measured from day 1 (D1), which was the first day the patient received the first dose of AMG 160
²Cohort enrolling

At the time of data analysis, 43 patients had received ≥1 dose of AMG 160 monotherapy at 6 target dose levels up to 0.9 mg, and 19 patients (44.2%) remained on treatment. Six patients received treatment for ≥6 months. Of the 43 men enrolled in the study, most (79.1%) were white. Mean age of patients was 66.0 years (range: 49 to 78 years) with baseline Eastern Cooperative Oncology Group (ECOG) status score of 0 or 1. Patients received a median of 4 prior lines of therapy (range: 1-9) with twenty-six subjects (60.5%) having received ≥4 prior lines of therapy.

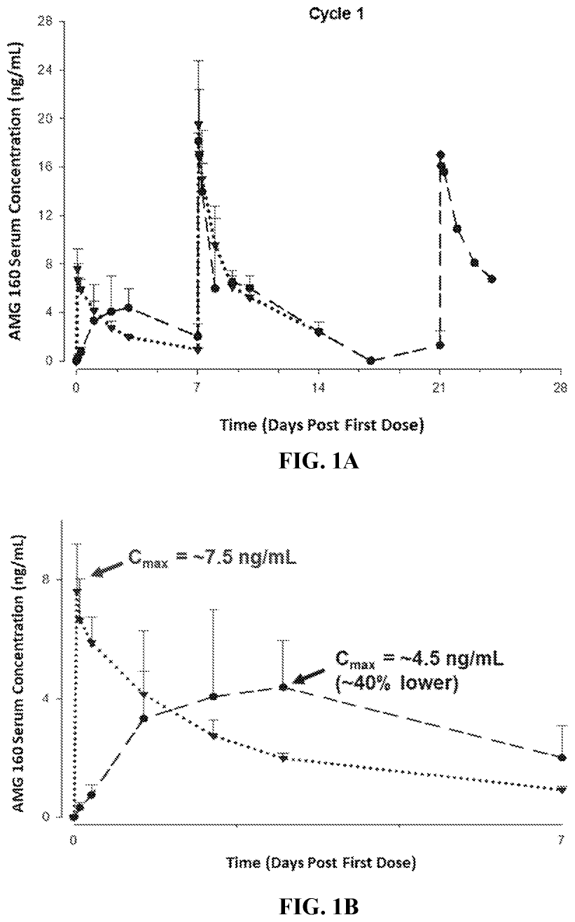

Preliminary serum pharmacokinetic (PK) profiles of AMG 160 for the first 14 days of cycle 1 were compared between patients with mCRPC in cohort 6b (two-step dose cohort) and cIV cohort 1. In cohort 6b, patients received a short-term IV infusion of AMG 160 at a dose of 0.03 mg on day 1 followed by a 0.09 mg dose on day 8 of cycle 1. In cIV cohort 1, patients received the same 0.03 mg first dose as patients in cohort 6b but administered over 3 days at a constant rate (e.g. 0.01 mg/day for 3 days) and the same 0.09 mg dose administered by short-term IV infusion at day 8 of cycle 1. Thus, comparison of serum PK profiles of these two cohorts allows for a direct comparison of the difference in serum exposure of AMG 160 for the same priming doses administered by the two different infusion approaches during the first week. As shown in FIGS. 1A and 1, when the 0.03 mg dose is given as a continuous IV infusion over 3 days rather than as a 60-minute infusion, the peak serum concentration (Cₘₐₓ) for the same dose of AMG 160 is about 40% lower (4.48 ng/mL vs. 7.49 ng/mL) and occurs about 72 hours after the start of infusion rather than about 1 hour after the start of infusion.

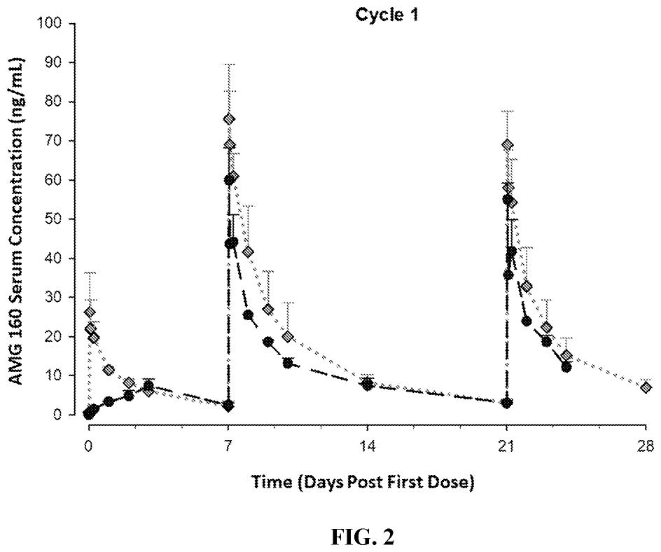

Patients in cohort 5 and patients in cIV cohort 2a both received a target dose of 0.3 mg of AMG 160. Patients in cohort 5 were escalated to this target dose by administering two dose steps of 0.01 mg and 0.09 mg on days 1 and 8, respectively, until receiving the 0.3 mg target dose on day 15. See Table 1. In contrast, patients in cIV cohort 2a received the 0.3 mg target dose on day 8 following administration of a first dose (e.g. priming dose) of 0.09 mg over days 1 to 3 as a continuous IV infusion (Table 1). Patients in both cohorts subsequently received the 0.3 mg target dose once every 14 days. The preliminary serum PK profiles for these two dosing cohorts are shown in FIG. 2. For comparison purposes, the AMG 160 serum concentrations for cohort 5 are shown starting with the administration of the second step dose of 0.09 mg and adjusted to start at day 0 in the graph. Similar to the comparison between dosing cohorts 6b and cIV cohort 1, administration of the same dose, in this case 0.09 mg, by cIV infusion over 3 days produces a reduced Cₘₐₓ as compared to the same dose administered by a 1-hr infusion (FIG. 2). In addition, a similar serum exposure is attained upon administration of the 0.3 mg target dose; however, the target dose is able to be administered 1 week earlier when the first dose is administered by continuous IV.

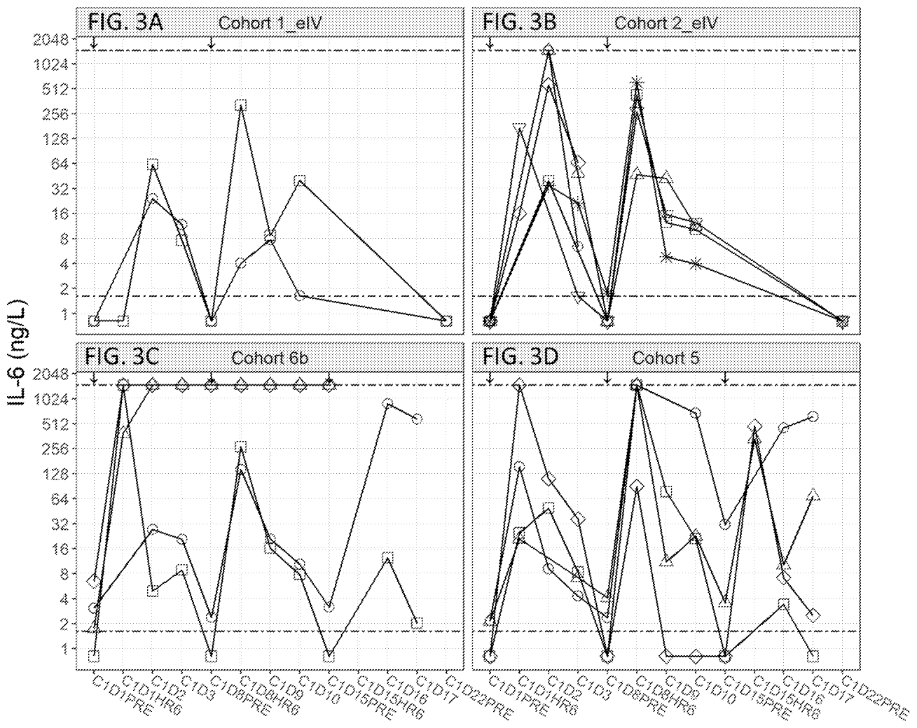

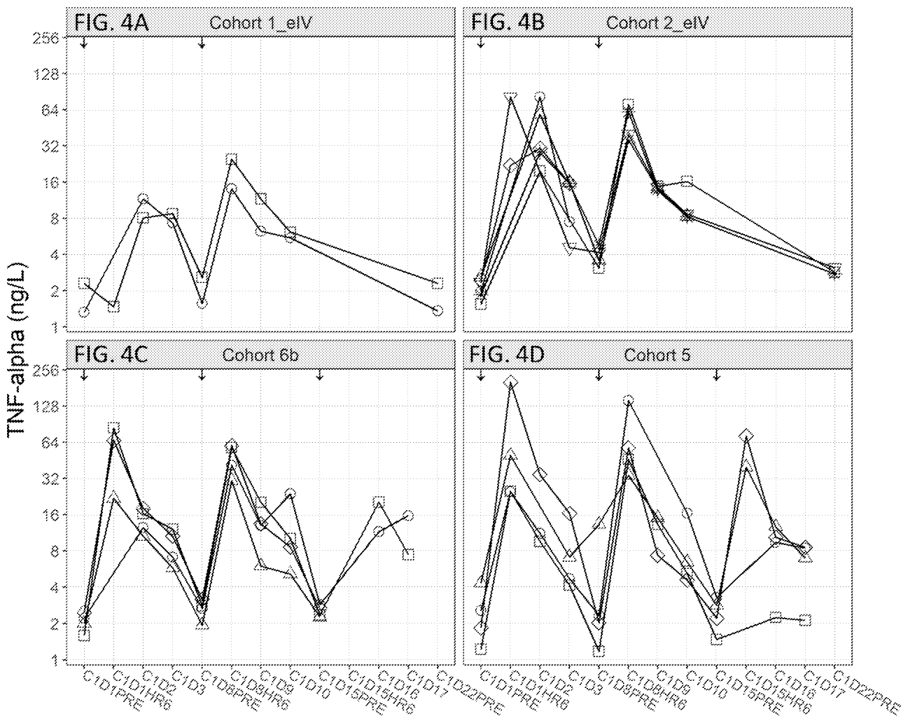

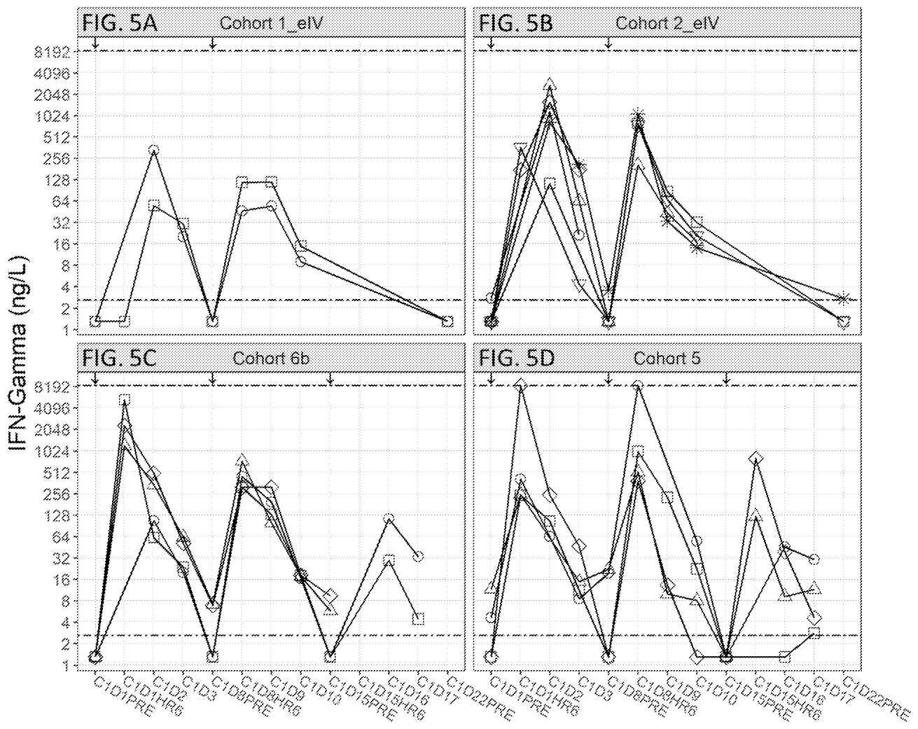

Serum levels of IL-6 (FIG. 3), TNF-alpha (FIG. 4), and IFN-gamma (FIG. 5) at various time points through the first 21 days of cycle 1 were compared between patients in cIV cohorts 1 and 2 and step-dosing cohorts 5 and 6b. When patients received the 0.03 mg priming dose of AMG 160 as a continuous IV infusion over 3 days as in cIV cohort 1, initial peak IL-6 levels were reduced as compared to when patients received the 0.03 mg priming dose as a 60-minute infusion as in cohort 6b (compare FIG. 3A to FIG. 3C). Also, IL-6 release was delayed from 6 hours to 24 hours in patients receiving the priming dose by a continuous IV infusion as compared to patients receiving the priming dose by a 60-min IV infusion. Similar results were observed for TNF-alpha and IFN-gamma levels; the initial peak levels of these two cytokines were reduced and delayed in patients receiving the 0.03 mg priming dose by a continuous IV infusion over 3 days as compared to the levels of these cytokines in patients receiving the 0.03 mg priming dose as a 60-minute IV infusion (compare FIG. 4A to FIG. 4C for TNF-alpha and FIG. 5A to FIG. 5C for IFN-gamma).

A comparison of patients in cIV cohorts 2a and 2b (combined as cohort 2 eIV in FIGS. 3B, 4B, and 5B), who received a 0.09 mg dose by continuous infusion over 2 to 3 days as the first AMG 160 dose, to patients in cohort 5, who received a first priming dose of 0.01 mg of AMG 160 on day 1 as a 60-minute infusion, shows that the continuous infusion of an initial 0.09 mg dose induced a similar release of IL-6, TNF-alpha, and IFN-gamma as patients who received a 9-fold lower dose of 0.01 mg as a short-term IV infusion (compare FIG. 3B to FIG. 3D for IL-6, FIG. 4B to FIG. 4D for TNF-alpha, and FIG. 5B to FIG. 5D for IFN-gamma). As was observed for patients in cIV cohort 1, the release of cytokines was delayed from 6 hours to 24 hours in some patients when the first dose of AMG 160 was administered by continuous infusion over 2 to 3 days. See FIGS. 3B, 4B, and 5B.

Treatment-emergent adverse events were reported for 41 subjects (95.3%) at the time of data analysis. There were no grade 5 events and no events resulting in treatment discontinuation. Three reversible dose-limiting toxicities occurred: grade 3 rash (n=2) and grade 3 GI hemorrhage (n=1). The most common adverse event was CRS, which presented with fever, transient transaminitis, hypotension, nausea/vomiting, and/or diarrhea, and occurred in 39 patients (any grade). CRS events were graded according to the Lee criteria as described in Lee et al., Blood, Vol. 124: 188-195, 2014. CRS was reversible and occurred primarily in cycles 1 and 2. Twenty-six patients (60.5%) had grade 2 CRS as worst grade and eleven patients (25.6%) had grade 3 CRS as worst grade. There were no grade 4 or 5 CRS events. Six out of thirty patients (20.0%) assessed at the time of data analysis developed anti-drug antibodies affecting exposure of AMG 160 between cycles 1 and 10. No adverse events clearly associated with the anti-drug antibodies were observed.

Table 2 below summarizes the safety and efficacy profile for the two-step, three-step, and cIV priming cohorts. Generally, the cIV priming cohorts exhibited an improved safety profile as compared to the cohorts receiving a step dosing regimen. For example, comparison of the two-step dosing cohort 6b to cIV cohort 1 reveals that administration of the same first dose (e.g. priming dose) of AMG 160 by a continuous IV infusion over 3 days rather than as a 60-min infusion avoided the occurrence of dose-limiting toxicities, serious adverse events, and dose reductions as well as reducing the number of grade 2 and grade 3 CRS events. Comparison of cohort 5 (two-step dose cohort) to cIV cohort 2a, in both of which patients receive a target dose of 0.3 mg, shows that administration of the priming dose of AMG 160 by a continuous IV infusion over 3 days eliminated the occurrence of serious adverse events and grade 3 CRS events. Similarly, comparison of cIV cohorts 3a and 3b, in which patients received a priming dose of AMG 160 administered by continuous IV infusion over 2 days or 3 days and then received a target dose of 0.9 mg, to any of cohorts 6a to 6c, in which patients are escalated to a target dose of 0.9 mg employing two or three dosing steps, shows that administration of the priming dose by continuous infusion over days reduces the number of serious adverse events as well as the number and severity of CRS events. As shown by a comparison of the safety measures between cohorts 2a and 2b, fewer dose reductions, serious adverse events, and grade 3 CRS events were observed if the same priming dose (e.g. 0.09 mg) was continuously infused over 3 days rather than 2 days.

TABLE 2
Summary of Safety and Efficacy of Step-Dosing and cIV Priming Cohorts¹

| | Cohort | Safety Measures | Efficacy Measures |
|---|---|---|---|
| Step-Dosing | Cohort 5 (n = 4) | No DLTs | 1 PSA30; 1 CTC0 |
| Regimens | two-step; | 4 SAEs | 1 PSA50 |
| | 0.3 mg TD | Grade 2 CRS (n = 3) | 1 SD |
| | | Grade 3 CRS (n = 2) | |
| | Cohort 6a (n = 4) | 1 DLT | 1 PSA50 |
| | two-step; 0.9 mg TD | 1 SAE | (unconfirmed) |
| | | 3 dose reductions | 1 PSA70 |
| | | Grade 2 CRS (n = 3) | 1 CTC0 |
| | | Grade 3 CRS (n = 4) | 1 SD |
| | Cohort 6b (n = 4) | 1 DLT | 1 PSA30 |
| | two-step; 0.9 mg TD | 5 SAEs | (unconfirmed) |
| | | 4 dose reductions | |
| | | Grade 2 CRS (n = 6) | |
| | | Grade 3 CRS (n = 4) | |
| | Cohort 6c (n = 4) | No DLTs | 1 PSA30 |
| | three-step; 0.9 mg TD | 4 SAEs | 1 PSA50 |
| | | No dose reductions | (unconfirmed) |
| | | Grade 2 CRS (n = 10) | 1 SD |
| | | No Grade 3 CRS | |
| cIV Priming | cIV cohort 1 (n = 2) | No DLTs | 1 PSA70 |
| Regimens | 0.09 mg TD | No SAEs | |
| | | No dose reductions | |
| | | No Grade 2 CRS | |
| | | Grade 3 CRS (n = 1; | |
| | | transaminitis only) | |
| | cIV cohort 2a (n = 3) | No DLTs | 2 PSA70 (1 |
| | 0.3 mg TD | No SAEs | unconfirmed) |
| | | No dose reductions | 1 PR (unconfirmed) |
| | | Grade 2 CRS (n = 4) | 2 SD |
| | | No Grade 3 CRS | |
| | cIV cohort 2b (n = 4) | No DLTs | 2 PSA70 |
| | 0.3 mg TD | 1 SAE | (unconfirmed) |
| | | 2 dose reductions | 2 PSA measurements |
| | | Grade 2 CRS (n = 4) | pending |
| | | Grade 3 CRS (n = 2) | Scans pending |
| | cIV cohort 3a and 3b | 1 DLT | 1 PSA70 |
| | (n = 3) | No SAEs | (unconfirmed) |
| | 0.9 mg TD | 2 dose reductions | 1 PSA90 |
| | | Grade 2 CRS (n = 3) | (unconfirmed) |
| | | Grade 3 CRS (n = 1) | Scans pending |

¹TD = target dose; DLT = dose limiting toxicity; SAE = serious adverse event; PR = partial response; SD = stable disease; CRS = cytokine release syndrome events

At the time of data analysis, preliminary evidence of efficacy and clinical benefit of AMVG 160 were observed in some patients. RECIST 1.1 responses among patients with measurable disease included 3 partial responses (PR; at target doses of 0.03 mg, 0.09 mg, and 0.3 mg in cohorts 3, 4, and cIV cohort 2a, respectively), 8 stable disease (SD), and 5 progressive disease (PD). PSA reductions occurred in 24 of 35 evaluable patients (68.6%). Evaluable patients included those who had received ≥1 dose of AMG 160 and had measurable baseline PSA levels. PSA reductions >50% as a best response occurred in 12 out of 35 (34.3%) evaluable patients. Overall, 8 patients out of 29 patients with 2 postbaseline PSA results (27.6%) had confirmed PSA responses: 1 PSA90 (0.09 mg target dose), 2 PSA70 (target doses of 0.09 mg and 0.9 mg), 2 PSA50 (target doses of 0.03 mg and 0.3 mg), and 3 PSA30 (target doses of 0.03 mg, 0.3 mg, and 0.9 mg). An additional 4 patients out of 35 (11.4%) patients with measurable PSA levels at baseline had unconfirmed PSA responses at the time of data analysis: 1 PSA70 (0.3 mg target dose), 2 PSA50 (0.9 mg target dose), and 1 PSA30 (0.9 mg target dose). Three patients out of 13 patients with baseline CTC >0 and postbaseline CTC assessment (23.1%) had a CTC0 response. Following this initial data cut, 4 more PSA70 responses and 1 PSA90 response (all unconfirmed) as well as 2 SD responses in RECIST 1.1 measurable patients were reported for the cIV priming cohorts. These responses as well as the other efficacy measures for the cIV priming cohorts and the two-step and three-step dosing cohorts are summarized in Table 2 above. Comparison of the efficacy results reported to date from the step-dosing cohorts to those from the cIV priming cohorts in which the same target dose was administered shows that patients in the cIV priming cohorts have an improved response with AMG 160. Specifically, patients escalated to a target dose of 0.3 mg from a priming dose administered by continuous IV infusion over 2-3 days (cIV cohorts 2a and 2b) had 4 PSA70 responses out of 5 patients with PSA measurements and 1 PR and 2 SD in patients with RECIST 1.1 measurable disease, whereas patients escalated to a target dose of 0.3 mg via two step doses of 0.01 mg and 0.09 mg (cohort 5) had 1 PSA30/CTC0 response in one patient and 1 PSA50 response/SD response in a second patient out of four patients in the cohort. The improved efficacy observed with cIV priming may be in part due to the ability to dose patients with the target dose earlier in cycle 1 than with step dosing due to the improved tolerability profile (e.g. reduction in CRS and adverse events) achieved with cIV priming.

To evaluate the effect of a longer infusion period for the priming dose, a separate cohort of patients (n=4) received a priming dose of 0.15 mg of AMG 160 by continuous IV infusion over 5 days (i.e. days 1 to 5 of cycle 1; 0.03 mg/day for 5 days) followed by a 0.3 mg target dose administered by short-term IV infusion (approx. 60 min) on day 8 and day 22 in cycle 1. Patients received the 0.3 mg target dose by short-term IV infusion on days 1 and 15 of cycle 2 and all other subsequent cycles. Out of the four patients enrolled in this cohort to date, 1 patient had a grade 3 CRS event, 2 patients had grade 2 CRS events, and 1 patient had a grade 1 CRS event as worst grade. Of the three patients evaluable at the time of data analysis, there was 1 patient that had a PSA90 response with stable disease by RECIST 1.1.

### Dose Expansion

AMG 160 was administered in a dose expansion cohort according to the same cIV dosing regimen as for cIV cohort 2a described above (see Table 1). Specifically, patients enrolled in the dose expansion cohort received a first dose (e.g. priming dose) of 0.09 mg by continuous IV infusion over days 1 to 3 (e.g. 0.03 mg/day for 3 days) followed by a 0.3 mg target dose administered by short-term IV infusion (approx. 60 min) on day 8 and every two weeks thereafter in cycle 1. Patients received the 0.3 mg target dose by short-term IV infusion on days 1 and 15 of cycle 2 and all other subsequent cycles.

As of the data cutoff date, 43 patients were enrolled in the dose expansion cohort and 40 patients received at least 1 dose of AMG 160. Enrolled patients had a median of four prior lines of therapy with twenty-four patients (60.0%) having received ≥4 prior lines of therapy. Patients also had an ECOG status score of 0 or 1 at baseline (i.e. prior to receiving AMG 160). Of the 43 enrolled patients, 18 (41.9%) discontinued treatment due to disease progression (13 patients), subject request (2 patients), adverse events (2 patients), or other reasons (1 patient).

In the dose expansion cohort at the time of the data cutoff date, adverse events considered by the site investigator to be related to the investigational product were reported for 38 patients (95%) with no treatment-related grade 5 events. Treatment-related adverse events reported for ≥20% patients were CRS (37 patients, 92.5%); nausea (19 patients, 47.5%); diarrhea (16 patients, 40%); dry mouth (15 patients, 37.5%); vomiting and fatigue (13 patients, 32.5% each); pyrexia (12 patients, 30%); decreased appetite (10 patients, 25%); rash (11 patients, 27.5%); dysgeusia (9 patients, 22.5%); and rash maculo-papular (8 patients, 20%). The most commonly reported grade 3 treatment-related adverse event was CRS (6 patients, 15%). Serious adverse events were reported for 22 patients (55%). The most commonly reported serious adverse events by system organ class were immune system disorders (12 patients, 30%). Serious adverse events by preferred term reported for ≥2 patients were CRS (12 patients, 30%), and general physical health deterioration (2 patients, 5%), and pain (2 patients, 5%). Twenty patients (50%) had serious adverse events considered by the site investigator to be related to AMG 160. Of these, 1 patient (2.5%) had grade 4 serious adverse events (CRS and acute kidney injury), and 12 patients (30%) had CTCAE grade 3 serious adverse events (CRS, AST increased, platelet count decreased, vomiting, anemia, disseminated intravascular coagulation, general physical health deterioration, deafness, and infection). Two patients (6.5%) in the dose expansion cohort at the time of the data cut had dose limiting toxicities, which included 1 subject with grade 3 serious event of AST increase that resolved in 3 days and another subject who had a grade 4 serious event of acute kidney injury (>7 days duration) which led to discontinuation.

Thirty-seven patients (92.5%) had grade 1 to 4 CRS as worst grade (there were no grade 5 CRS events). One patient (2.5%) had a grade 4 CRS event, 6 patients (15%) had grade 3 CRS events, 27 patients (67.5%) had grade 2 CRS events, and 29 patients (72.5%) had grade 1 CRS events as worst grade. The most commonly reported CRS symptoms in ≥20% of patients included fever, nausea, hypotension, elevated liver enzymes (aspartate aminotransferase (AST), alanine aminotransferase (ALT), and gamma-glutamyl transferase (GGT)), vomiting, and diarrhea, fatigue, tachycardia, rigors, elevated alkaline phosphatase (ALP), hypoxia, and anorexia. CRS was most severe with 1^{st} and 2^{nd} doses and was reversible and manageable with standard treatment approaches (e.g., tocilizumab, corticosteroids, and vasopressors). As compared to step-dosing cohorts 5, 6a, 6b, and 6c (see Table 1 above), there were fewer dose reductions and grade 3 CRS events in the dose expansion cohort using a cIV priming regimen (data not shown), indicating that the cIV priming approach improves the tolerability profile for AMG 160.

As of the date of the data cut off, preliminary evidence of efficacy and clinical benefit of AMG 160 in the dose expansion cohort were observed in some patients. In terms of PSA reductions, 88% of patients experienced at least some level of PSA decline. Of the 34 evaluable patients, 12 (35.3%) had a confirmed PSA reduction ≥30%, 9 (26.5%) had a confirmed PSA reduction ≥50%, 7 (20.6%) had a confirmed PSA reduction ≥70%, and 3 (8.8%) had a confirmed PSA reduction ≥90%. Sixteen patients out of the 40 patients who received at least one dose of AMG 160 at the time of data analysis had RECIST-measurable disease. RECIST 1.1 responses among the 12 patients (75%) with postbaseline response assessments included 6 patients (37.5%) with stable disease, 3 patients (18.8%) with unconfirmed partial responses, and 3 patients (18.8%) with unconfirmed progressive disease. Gallium PSMA-11 response (50% SUVₘₐₓ decrease) was reported for 4 patients (12.9%). Most of the AMG 160-treated patients had reduction in LDH levels, a marker of tumor burden (97.5% patients), and ALP levels, an indicator of bone disease (95% patients), with ≥50% reduction in LDH and ALP levels reported in 27.5% and 17.5% patients, respectively.

The results described in this Example demonstrate that administration of the first dose of AMG 160 in cycle 1 (i.e. the priming dose) by a continuous IV infusion over 2 to 3 days reduces the peak serum concentration (Cₘₐₓ) of AMG160 and delays the time to Cₘₐₓ by 2-3 days as compared to administration of the priming dose by a short IV infusion (e.g. 60 min infusion). This PK profile was associated with reduced initial IL-6, TNF-alpha, and IFN-gamma release in some patients. Patients receiving the AMG 160 priming dose by a 2—to 3-day continuous infusion exhibited a reduced number of serious adverse events, dose reductions, and grade 2 and 3 CRS events as compared to patients receiving a step-dosing regimen of AMG 160 in which each of the step doses was administered by a 60-min IV infusion. Patients in cIV priming cohorts also exhibited better efficacy responses in terms of PSA reductions and RECIST measurable responses than patients receiving the same target dose administered by a step-dosing regimen.

### Example 2. Cycle 1 Priming Dose Regimens for a BCMA×CD3 Bispecific T-Cell Engaging Molecule in Patients with Multiple Myeloma

AMG 701 is an HLE BiTE® molecule that binds both B-cell maturation antigen (BCMA) and CD3 and comprises a single chain IgG Fc domain. The amino acid sequence of AMG 701 is set forth in SEQ ID NO: 50. This study is a phase 1 open-label, dose-exploration study to evaluate the safety, tolerability, and efficacy of AMG 701 in patients who have relapsed/refractory multiple myeloma.

After signing informed consent, patients enter the screening period (up to 21 days), during which eligibility of the patients is assessed. Eligible patients are patients ≥18 years of age who have multiple myeloma relapsed after and/or refractory to established and available therapies with known clinical benefit, including a proteasome inhibitor, an immunomodulatory drug, and a CD38-directed antibody. Key patient inclusion criteria include:
Multiple myeloma meeting the following criteria:
   Pathologically-documented diagnosis of multiple myeloma that is relapsed or is refractory as defined by the following:
      Relapsed after ≥3 lines of prior therapy that must include a proteasome inhibitor (PI), an immunomodulatory drug (IMiD), and a CD38-directed antibody in combination in the same line or separate lines of treatment; or
      Refractory to PI, IMiD and CD38-directed antibody
      Refractory multiple myeloma is defined as disease that is nonresponsive (i.e. failure to achieve a minimal response) while on primary or salvage therapy or progresses within 60 days of last therapy.
      Relapsed multiple myeloma is defined as previously treated multiple myeloma that progresses and requires the initiation of salvage therapy but does not meet the criteria for refractory multiple myeloma.
   Measurable disease, defined by 1 or more of the following at time of screening:
      a serum M protein ≥0.5 g/dL measured by serum protein electrophoresis
      urinary M protein excretion ≥200 mg/24 hours
      involved serum free light chains (sFLC) measurement >10 mg/dL, provided that the sFLC ratio is abnormal as per International Myeloma Working Group (IMWG) response criteria
Eastern Cooperative Oncology Group (ECOG) Performance Status of ≤2
Life expectancy of at least 3 months as per investigator's judgment at time of screening
Hematological function without transfusion support as follows:
   absolute neutrophil count (ANC) ≥1.0×10⁹/L (without growth factor support)
   platelet count ≥50×10⁹/L (without transfusions within 7 days from screening assessment)
   hemoglobin ≥8.0 g/dL (transfusions permitted no later than 48 hours before screening)
Renal function as defined by a calculated or measured creatinine clearance ≥30 mL/min using the Cockcroft-Gault equation or via 24-hour urine collection with plasma and urine creatinine concentrations; and
Hepatic function as follows:
   aspartate aminotransferase (AST) and alanine aminotransferase (ALT)<3×upper limit of normal (ULN)
   total bilirubin (TBIL)<1.5×ULN (unless considered due to Gilbert's syndrome)

The first dose (e.g. priming dose) of AMG 701 is administered as a continuous IV infusion over the course of 2 or 7 days during the first week of cycle 1 followed by weekly short-term IV infusions (e.g. 60-minute IV infusions) of the target dose of AMG 701 beginning on day 8 of the cycle. AMG 701 is administered in 28-day cycles and the date of the first dose of AMG 701 is defined as day 1 in the cycle. Administration of AMG 701 by continuous IV infusion during the first week of cycle 1 is designed to achieve efficacious exposure levels of AMG 701 as early as possible in cycle 1 and within the ranges of those previously observed when AMG 701 was administered on a weekly dosing interval. Without being bound by theory, these continuous IV priming dosing regimens are believed, based on PK simulations, to achieve the serum free AMG 701 projected efficacious exposures within 2 to 4 days, but more importantly they are also predicted to avoid any rapid increase in free AMG 701 serum exposures as seen with short-term 60-minute IV infusions with a sharp increase in free AMG 701 serum concentrations, e.g. a peak serum concentration (Cₘₐₓ) within 1 hour of infusion start. A slow ramp up in free AMG 701 concentrations and delaying the time to Cₘₐₓ is believed to reduce the risk of CRS. These cIV priming dosing regimens are believed to enable optimal T cell engagement of target cells during week 1, without rapid increases in serum concentrations of free AMG 701, which have been associated with induction of Grade 2 and higher CRS following initial cycle 1 doses of AMG 701 administered by 60-min IV infusions.

In two cohorts, patients receive a first dose (e.g. a priming dose) of AMG 701 administered by continuous infusion over a period of 2 days (cycle 1 days 1-2), followed by a short-term IV infusion (e.g. 60-min infusion) of a boost dose on cycle 1 day 3, followed by administration of the target dose as a short-term IV infusion on cycle 1 day 8, 15, and 22 of the 28-day cycle. In two other cohorts, patients receive a first dose (e.g. a priming dose) of AMG 701 administered by continuous infusion over a period of 7 days (cycle 1 days 1-7) followed by administration of the target dose as a short-term IV infusion on cycle 1 day 8, 15, and 22 of the 28-day cycle. After cycle 1, cycle 2 and all subsequent cycles entail the administration of the target dose as a short-term IV infusion (e.g. approx. 60 min) of AMG 701 on days 1, 8, 15, and 22 of the 28-day cycle. The priming dose of AMG 701 is administered at a constant rate over the indicated period of days (e.g. over 2 or 7 days). For example, for a priming dose of 8.4 mg administered over 7 days, the priming dose is infused continuously at a constant rate to deliver 1.2 mg/day for 7 days. Similarly, for a priming dose of 4.6 mg administered over 2 days, the priming dose is infused continuously at a constant rate to deliver 2.3 mg/day for 2 days.

Patients are dosed in each of four cohorts as follows:
Cohort 1: priming dose of 8.4 mg administered by continuous IV infusion over 7 days (e.g. 1.2 mg/day for 7 days) on cycle 1 day 1 to day 7 followed by administration of a target dose of 12 mg as a short-term IV infusion (e.g. 60-minute IV infusion) on cycle 1 day 8, 15, and 22
Cohort 2A: priming dose of 16.1 mg administered by continuous IV infusion over 7 days (e.g. 2.3 mg/day for 7 days) on cycle 1 day 1 to day 7 followed by administration of a target dose of 12 mg to 18 mg as a short-term IV infusion (e.g. 60-minute IV infusion) on cycle 1 day 8, 15, and 22
Cohort 2B: priming dose of 4.6 mg administered by continuous IV infusion over 2 days (e.g. 2.3 mg/day for 2 days) on cycle 1 day 1 to day 2, followed by administration of a boost dose of 0.8 mg as a short-term IV infusion (e.g. 60-minute IV infusion) on cycle 1 day 3, followed by administration of a target dose of 12 mg to 18 mg as a short-term IV infusion (e.g. 60-minute IV infusion) on cycle 1 day 8, 15, and 22
Cohort 3: priming dose of 9.2 mg administered by continuous IV infusion over 2 days (e.g. 4.6 mg/day for 2 days) on cycle 1 day 1 to day 2, followed by administration of a boost dose of 1.6 mg as a short-term IV infusion (e.g. 60-minute IV infusion) on cycle 1 day 3, followed by administration of a target dose of 12 mg to 18 mg as a short-term IV infusion (e.g. 60-minute IV infusion) on cycle 1 day 8, 15, and 22

Cohort 2A and/or Cohort 2B is selectively opened only after review of all available safety, PK, and pharmacodynamic (PD) data from Cohort 1. Cohort 3 is only opened after review of all available safety, PK, and PD data from Cohorts 2A and/or 2B. Each cohort enrolls from 4 to 7 eligible patients. Prior to the start of AMG 701 infusions in cycle 1, unless contraindicated in the patient, a glucocorticoid at an equivalent dose to 50 mg prednisone, 40 mg methylprednisone, or 8 mg dexamethasone is intravenously administered to the patient within 1 hour of administration of each dose of AMG 701 in cycle 1. Prior to the first dose of AMG 701 in cycle 2, if CRS >grade 1 occurs with administration of the preceding dose, 8 mg dexamethasone or equivalent dose of glucocorticoid is administered intravenously to the patient within 1 hour of the first dose of AMG 701 in cycle 2. Otherwise, 4 mg dexamethasone (equivalent to 25 mg prednisone or 20 mg methylprednisone) is administered intravenously to the patient within 1 hour of the first dose of AMG 701 in cycle 2.

Efficacy of AMG 701 is evaluated by the overall response according to IMWG response criteria (see Kumar et al., Lancet Oncol., Vol. 17: e328-346, 2016) and best overall response in each response category: stringent complete response (sCR), complete response (CR), very good partial response (VGPR), and partial response (PR). The IMWG response criteria for each category of response are as follows:
Complete response (CR):
   Negative M protein immunofixation on the serum and urine,
   Disappearance of any soft tissue plasmacytomas, and
   <5% plasma cells in bone marrow (BM) aspirates
   In patients with baseline measurable disease only by sFLC, a normal FLC ratio is required
Stringent complete response (sCR):
   CR as defined above,
   Normal FLC ratio,
   Absence of clonal cells in BM biopsy by immunohistochemistry (κ/λ ratio ≤4:1 or ≥1:2 for κ and λ patients, respectively, after counting ≥100 plasma cells)
Very good partial response (VGPR):
   Serum and urine M-protein detectable by immunofixation but not on electrophoresis or ≥90% reduction in serum M-protein plus urine M-protein level <100 mg/24 hrs
   In patients with baseline measurable disease only by sFLC, a ≥90% decrease in the difference between involved and uninvolved FLC levels is required in place of the M-protein criteria
   In patients achieving a VGPR by other criteria, a soft tissue plasmacytoma must decrease by more than 90% in the sum of the products of the maximal perpendicular diameters of measured lesions (SPD) compared with baseline
Partial response (PR):
   ≥50% reduction of serum M-protein and reduction in 24-hour urinary M-protein by ≥90% or to <200 mg/24 hrs
   In patients with baseline measurable disease only by sFLC, a ≥50% decrease in the difference between involved and uninvolved FLC levels is required in place of the M-protein criteria
   If serum and urine M-protein are not measurable, and serum free light assay is also not measurable, ≥50% reduction in plasma cells is required in place of M-protein, provided baseline BM plasma cell percentage was ≥30%
   If present at baseline, a ≥50% reduction in the size (SPD) of soft tissue plasmacytomas is also required

Adverse event and serious adverse event as well as disease-related event assessments are made throughout the study and are evaluated and recorded in the source documents. The severity of all events is graded according to CTCAE, version 4.0. However, CRS is graded according to the Lee criteria described in Lee et al., Blood, Vol. 124: 188-195, 2014. Briefly, the CRS grading that is used in this study is described in Table 3 below:

TABLE 3
Grading of Cytokine Release Syndrome

| CRS | |
|---|---|
| Grade | Description of Severity |
| 1 | Symptoms are not life-threatening and may include fever, |
| | nausea, fatigue, and require symptomatic treatment only |
| | Includes no higher than Grade 2 transaminitis and no higher than |
| | Grade 1 organ toxicity (including CRS-associated neurotoxicity |
| | events) per CTCAE criteria |
| 2 | Symptoms require and respond to moderate intervention: |
| | Oxygen requirement <40%, or |
| | Hypotension responsive to fluids or low dose of 1 |
| | vasopressor, or |
| | Grade 2 organ toxicity (including CRS-associated |
| | neurotoxicity events) or grade 3 transaminitis per CTCAE |
| | criteria |
| 3 | Symptoms require and respond to aggressive intervention |
| | Oxygen requirement ≥40%, or |
| | Hypotension requiring high dose or multiple vasopressors, or |
| | Grade 3 organ toxicity (including CRS-associated |
| | neurotoxicity events) or grade 4 transaminitis per CTCAE |
| | criteria |
| 4 | Life-threatening symptoms |
| | Requirement for ventilator support or |
| | Grade 4 organ toxicity (excluding transaminitis) per CTCAE |
| | criteria |

Four patients were enrolled in cohort 1 and received a priming dose of 8.4 mg of AMG 701 administered by continuous IV infusion over 7 days (e.g. 1.2 mg/day for 7 days) on cycle 1 day 1 to day 7 followed by administration of a target dose of 12 mg as a short-term IV infusion (e.g. 60-minute IV infusion) on cycle 1 day 8, 15, and 22. In cycle 2 and subsequent cycles, AMG 701 was administered at a target dose of 12 mg by short-term IV infusion on a weekly basis. Of the 4 patients enrolled in the cohort, 1 patient had a confirmed CR and remains on treatment in cycle 11 and 1 patient had a confirmed VGPR at cycle 3 but progressed at cycle 6. The remaining 2 patients did not complete cycle 1 due to adverse events. Two of the 4 patients in the cohort had grade 1 CRS events, whereas the other 2 patients had grade 2 CRS events.

### Example 3. Comparison of Cycle 1 Priming Dose Regimens for a CLDN18.2×CD3 Bispecific T-Cell Engaging Molecule

AMG 910 is an HLE BiTE® molecule that binds both claudin (CLDN)_{18.2}, an isoform of the cellular tight junction protein CLDN 18, and CD3 and comprises a single chain IgG Fc domain. The amino acid sequence of AMG 910 is set forth in SEQ ID NO: 160. AMG 910 is designed to redirect T cells toward CLDN18.2-expressing cells and kill them via T cell-mediated cytotoxicity. AMG 910 is currently under clinical investigation for treatment in adult subjects with metastatic or locally advanced unresectable gastric adenocarcinoma or gastroesophageal junction (GEJ) adenocarcinoma positive for CLDN18.2. This study is a phase 1 open label, dose exploration study to evaluate the safety, tolerability, pharmacokinetics, and pharmacodynamic effects of AMG 910 in patients with CLDN18.2+ gastric adenocarcinoma.

Patients with histologically or cytologically confirmed metastatic or locally advanced unresectable gastric adenocarcinoma or GEJ adenocarcinoma positive for CLDN18.2 who are refractory to or have relapsed after two or more prior lines of standard systemic therapy that included a platinum, a fluoropyrimidine, either a taxane or irinotecan, and an approved vascular endothelial growth factor receptor (VEGFR) antibody/tyrosine kinase inhibitor (TKI) were enrolled in the study. The dose exploration was conducted in 2 stages: single-patient cohorts followed by multiple patient cohorts (3 to 4 patients per cohort). AMG 910 was administered to patients in 28-day cycles and the date of the first dose of AMG 910 was defined as day 1 in the cycle.

For the single patient enrolled in cohort 1, a CRS grade 2 and abdominal pain grade 2 observation triggered switch from the single patient cohort to multiple patient cohorts. In the first multiple patient cohort, a target dose of AMG 910 was administered by a short-term IV infusion (e.g. approx. 60 min infusion) on each of days 1, 3, 8, 15, and 22 of cycle 1. In cycle 2 and all subsequent cycles, the target dose was administered weekly as a short-term IV infusion, i.e. on days 1, 8, 15, and 22 of each 28-day cycle. Of the 6 patients enrolled in this first multiple patient cohort 1, 5 were evaluable for dose-limiting toxicities (DLT). Two DLTs (grade 3 transaminitis and grade 3 atrial fibrillation) were observed in 2 of the 5 patients. The DLT of grade 3 atrial fibrillation was reported in the setting of grade 3 CRS. In addition, another patient experienced a grade 2 CRS event.

Cohort 1b, a cIV priming regimen, with the same target dose as cohort 1 was enrolled with 4 patients. In cohort 1b, the first dose (e.g. priming dose) of AMG 910 was administered as a continuous IV infusion over the course of 4 days (96 hours) starting on cycle 1 day 1 followed by administration of the target dose of AMG 910 by short-term IV infusion (approx. 60 min infusion) on each of days 8, 15, and 22 of cycle 1. The priming dose, which was the sum of the doses given on days 1 and 3 in the dosing regimen in cohort 1 (i.e. twice the target dose), was administered at a constant rate over the four-day period. In cycle 2 and all subsequent cycles, the target dose was administered weekly as a short-term IV infusion, i.e. on days 1, 8, 15, and 22 of each 28-day cycle. All 4 patients enrolled in cohort 1b completed dosing until day 8, at which time 1 patient discontinued treatment and the remaining 3 patients continued on to complete cycle 1 dosing. Two of the four patients developed grade 1 CRS only. No treatment-related grade 3 toxicity was reported for patients in cohort 1b during cycle 1 dosing. These results show that use of a cIV priming dosing approach in cycle 1 week 1 enabled the administration of a target dose of AMG 910 without eliciting grade 2 or higher CRS events or dose limiting toxicities and patients were better able to tolerate AMG 910 in general as compared to administration of the same target dose by short term IV infusions in cycle 1 week 1.

### Example 4. Continuous IV Priming Regimen for a Multispecific T-Cell Engaging Molecule

To evaluate whether a cIV priming regimen also reduces adverse events for other types of T-cell engaging molecules, a multispecific T-cell engaging molecule that binds two cancer cell antigens (cadherin 3 (CDH3) and mesothelin (MSLN)) and CD3 on T cells was administered to male cynomolgus monkeys according to two different dosing regimens. The CDH3×MSLN T-cell engaging molecule (CDH3×MSLN TCE) comprises a scFv domain binding to human CDH3, a scFv domain binding to human MSLN, two scFv domains binding to human CD3, and a single chain IgG Fc domain. The CDH3×MSLN TCE molecule was administered to monkeys in the following four different treatment groups:
Group 1 (n=2): 1000 μg/kg administered by slow intravenous injection (over approx. 2 minutes) on each of study days 1, 2, 3, 4, 5, 6, 7, 8, and 15 (daily dosing; dose level 1)
Group 2 (n=1): 5000 μg/kg administered by slow intravenous injection (over approx. 2 minutes) on each of study days 1, 2, 3, 4, 5, 6, 7, 8, and 15 (daily dosing; dose level 2)
Group 3 (n=2): 7000 μg/kg administered as a continuous IV infusion over the course of 7 days (i.e. from study days 1 to 7; 1000 μg/kg/day) and 1000 μg/kg administered by slow intravenous injection (over approx. 2 minutes) on each of study days 8 and 15 (cIV priming; dose level 1)
Group 4 (n=1): 35000 μg/kg administered as a continuous IV infusion over the course of 7 days (i.e. from study days 1 to 7; 5000 μg/kg/day) and 5000 μg/kg administered by slow intravenous injection (over approx. 2 minutes) on each of study days 8 and 15 (cIV priming; dose level 2)

Comparable serum exposures of CDH3×MSLN TCE were observed for animals between groups 1 and 3 (1000 μg/kg dose level) and between groups 2 and 4 (5000 μg/kg dose level), indicating that the pharmacokinetic profile for the molecule was similar between the two different dosing approaches (data not shown). Interestingly, fewer clinical signs of side effects were observed for animals that received CDH3×MSLN TCE using the cIV priming dosing regimen as compared to the daily dosing regimen (Table 4).

TABLE 4
Clinical Signs of CDH3 × MSLN TCE in Cynomolgus Monkeys

| 1000 μg/kg | | 5000 μg/kg | |
|---|---|---|---|
| | Group 3 | | Group 4 |
| Group 1* | (cIV | Group 2* | (cIV |
| (daily dosing) | priming) | (daily dosing) | priming) |
| Erected fur, hindpaw | No clinical | Erected fur, | No clinical |
| Skin flaking | signs | hindpaw | signs |
| Red skin | | Red skin | |
| Decreased activity | | Decreased activity | |
| Tremors | | Tremors | |
| Abnormal gait | | Reduced appetite | |
| Reduced appetite | | | |

*Clinical signs shown are from one animal in each of groups 1 and 2

Following daily dosing with CDH3×MSLN TCE, slightly erected fur was observed on hind paw for one animal in group 1 (1000 μg/kg/dose) and the one animal in group 2 (5000 μg/kg/dose) 2 hours post-dose on Day 1. On Day 2, the animal in group 1 presented with a transient abnormal gait 2 hours post-dose and a slight decreased activity associated with slight generalized tremors 4 hours post-dose. Similar clinical signs were also observed for the animal in group 2. On Day 3, red colored skin and red spots were noted pre-dose and up to 4 hours post-dose for both these animals in groups 1 and 2. On Day 4, slight desquamation and/or dry skin were observed on mouth, forepaws, hindpaws and scrotum of the animal in group 1, up to Day 8, and the animal in group 2 presented with slight red staining of inguinal fur up to Day 7. In addition, a transient reduced food consumption was noted for the cage the affected animal of group 1 was housed in, which was associated with a transient body weight loss for this animal only.

In contrast, no clinical signs were observed for animals in groups 3 and 4 who received a cIV priming regimen of the same doses of CDH3×MSLN TCE. One out of 2 animals in group 3 showed a moderate treatment-related decrease in body temperature on Day 1, 2 hours following start of infusion. This decrease was transient and the values returned close to baseline within 4 hours.

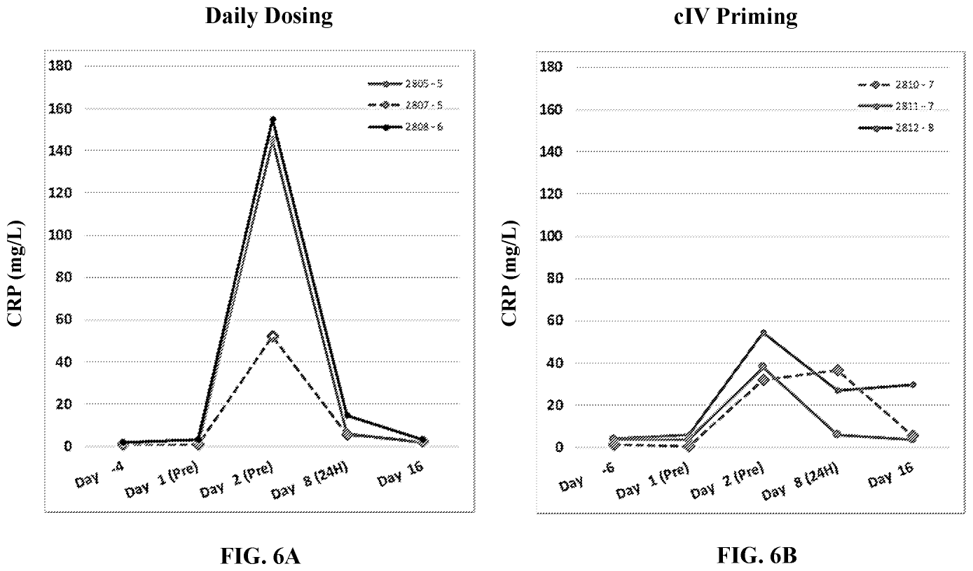

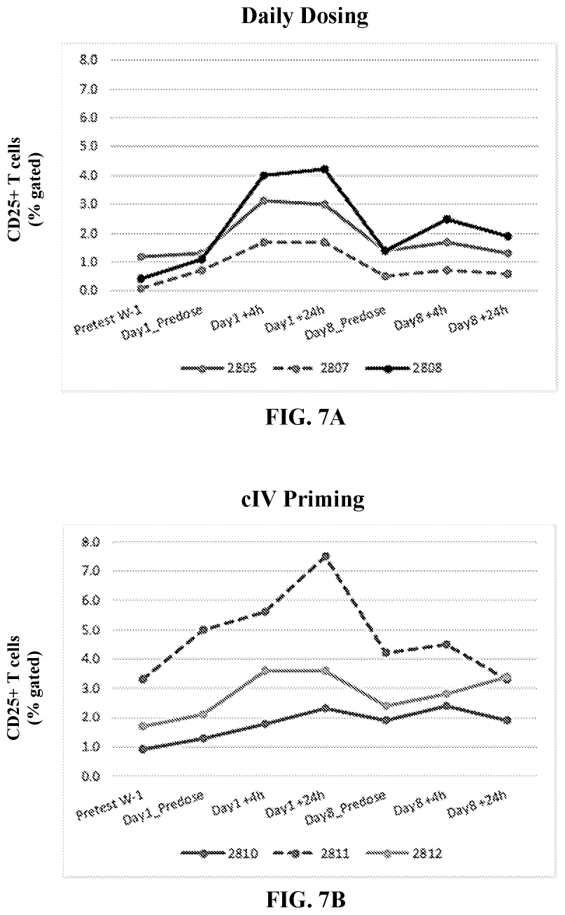

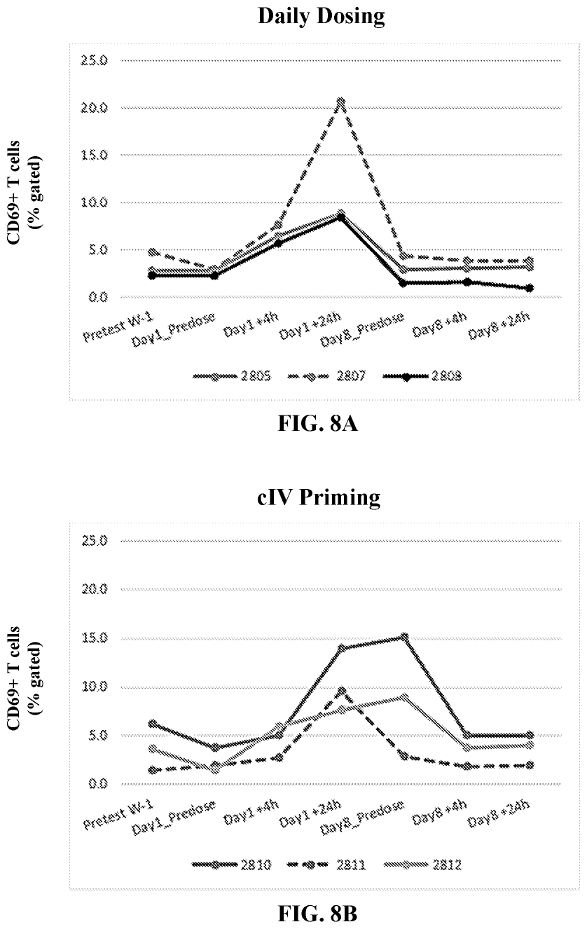

Indicators of the acute phase of the innate immune response were observed in all four groups, including (but not limited to): minimum to moderate increases in C-reactive protein (CRP) on day 2 (FIGS. 6A and 6B) and minimum to mild decreases in albumin and cholesterol on days 2 and 9 and persisting in individual animals on day 16 (data not shown). Values for CRP were considerably higher in the daily dosing groups (groups 1 and 2; FIG. 6A) as compared to the cIV priming groups (groups 3 and 4; FIG. 6B) at equivalent dose levels, suggesting a reduced level of inflammation. An increase in numbers of activated T cells, both CD25+ and CD69+ T-cell populations, were observed in all four groups indicative of the T-cell engaging activity of this molecule (FIGS. 7A, 7B, 8A, and 8B).

The results of this study show that administration of a multispecific T-cell engaging molecule using a cIV priming regimen, in which the first dose of the molecule is administered by a continuous IV infusion over the course of several days, induces fewer side effects as compared to administration of the molecule by daily slow IV injections, but produces comparable levels of T-cell activation.

### Example 5. Cycle 1 Priming Dose Regimen for a MUC17×CD3 Bispecific T-Cell Engaging Molecule in Patients with Gastrointestinal Cancer

AMG 199 is an HILE BiTE® molecule that binds both Mucin 17 (MUC17) and CD3 and comprises a single chain IgG Fc domain. The amino acid sequence of AMG 199 is set forth in SEQ ID NO: 171. This study is a phase 1 open-label, dose-exploration study to evaluate the safety, tolerability, and anti-tumor activity of AMG 199 in patients who have MUC17-positive gastric cancer or gastroesophageal junction cancer. Patients with histologically or cytologically confirmed metastatic or locally advanced unresectable gastric adenocarcinoma or gastroesophageal junction (GEJ) adenocarcinoma positive for MUC17 who are refractory to or have relapsed after two or more prior lines of standard systemic therapy that included a platinum, a fluoropyrimidine, either a taxane or irinotecan, and an approved vascular endothelial growth factor receptor (VEGFR) antibody/tyrosine kinase inhibitor (TKI) are enrolled in the study. AMG 199 is administered to patients in 28-day cycles and the date of the first dose of AMG 199 is defined as day 1 in the cycle. The following two dosing regimens are evaluated in separate cohorts of patients:
Dosing regimen #1: A target dose of AMG 199 is administered by a short-term IV infusion (e.g. approx. 60 min infusion) on each of days 1, 3, 8, 15, and 22 of cycle 1. In cycle 2 and all subsequent cycles, the target dose is administered weekly as a short-term IV infusion, i.e. on days 1, 8, 15, and 22 of each 28-day cycle.
Dosing regimen #2 (cIV priming): The first dose (e.g. priming dose) of AMG 199 is administered as a continuous IV infusion over the course of 4 days (96 hours) starting on cycle 1 day 1 followed by administration of the target dose of AMG 199 by short-term IV infusion (approx. 60 min infusion) on each of days 8, 15, and 22 of cycle 1. The priming dose, which is the sum of the doses given on days 1 and 3 in dosing regimen #1 (i.e. twice the target dose), is administered at a constant rate over the four-day period. In cycle 2 and all subsequent cycles, the target dose is administered weekly as a short-term IV infusion, i.e. on days 1, 8, 15, and 22 of each 28-day cycle.

Anti-tumor activity of AMG 199 is assessed by objective response per Response Evaluation Criteria in Solid Tumors (RECIST) 1.1 and iRECIST. Adverse event and serious adverse event as well as disease-related event assessments are made throughout the study and are evaluated according to CTCAE version 5.0. However, CRS is graded according to the Lee criteria described in Lee et al., Blood, Vol. 124: 188-195, 2014 (see, e.g., Table 3 above), and tumor lysis syndrome (TLS) is graded according to the Cairo Bishop criteria referenced in Coiffier et al., Journal of Clinical Oncology, Vol. 26: 2767-2778, 2008.

Administration of AMG 199 according to the cIV priming regimen is expected to induce a lower incidence of and/or reduced severity of CRS events in patients as compared to administration according to dosing regimen #1. Use of the cIV priming regimen is also expected to enable administration of a greater target dose than dosing regimen #1, which may enhance the anti-tumor efficacy of AMG 199.

### Example 6. Cycle 1 Priming Dose Regimen for a DLL3×CD3 Bispecific T-Cell Engaging Molecule in Patients with Small Cell Lung Cancer

AMG 757 is an HLE BiTE® molecule that binds both delta like ligand 3 (DLL3) and CD3 and comprises a single chain IgG Fc domain. The amino acid sequence of AMG 757 is set forth in SEQ ID NO: 40. This study is a phase 1 open-label, dose-exploration study to evaluate the safety, tolerability, and anti-tumor activity of AMG 757 in patients who have relapsed/refractory small cell lung cancer (SCLC).

Patients ≥18 years of age with histologically or cytologically confirmed SCLC who have progressed or recurred following at least one platinum-based regimen are enrolled in the study. AMG 757 is administered to patients in 28-day cycles and the date of the first dose of AMG 757 is defined as day 1 in the cycle. The first dose (e.g. priming dose) of AMG 757 is administered as a continuous IV infusion over the course of 3 days (72 hours) starting on cycle 1 day 1 followed by administration of the target dose of AMG 757 by short-term IV infusion (approx. 60 min infusion) on each of days 8 and 15 of cycle 1. The priming dose is about 30% to about 35% of the target dose and is administered at a constant rate over the three-day period. In cycle 2 and all subsequent cycles, the target dose is administered biweekly as a short-term IV infusion, i.e. on days 1 and 15 of each 28-day cycle. All patients are pre-treated with 8 mg PO dexamethasone 6-16 hours prior to all doses of AMG 757 in cycle 1. Additionally, dexamethasone 8 mg IV was administered within 1 hour prior to all doses of AMG 757 in cycle 1.

Anti-tumor activity of AMG 757 is assessed by contrast-enhanced MRI/CT and determining an objective response per modified Response Evaluation Criteria in Solid Tumors (RECIST) 1.1. Adverse event and serious adverse event as well as disease-related event assessments are made throughout the study and are evaluated according to CTCAE version 4.0, except that CRS is graded according to the Lee criteria described in Lee et al., Blood, Vol. 124: 188-195, 2014 (see, e.g., Table 3 above).

It is hypothesized that administration of the first dose (e.g. a priming dose) of AMG 757 by continuous intravenous infusion over a 72-hour period may reduce the intensity and/or frequency of the symptoms associated with CRS relative to the same total dose of AMG 757 when infused over a 60-minute duration. It is additionally hypothesized that such a cIV priming approach may help achieve higher cumulative average serum exposures of AMG 757 during the first week of treatment, relative to a step dosing paradigm, which may lead to enhanced pharmacodynamic activity.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize or be able to ascertain using no more than routine experimentation, many equivalents to the specific embodiments of the invention described herein. Such equivalents are intended to be encompassed by the following claims.

TABLE 5
Sequence Listing

| SEQ | | |
|---|---|---|
| ID | | |
| NO: | Description | Amino Acid Sequence |
| 1 | Anti-CD19 CDRH1 | SYGMH |
| 2 | Anti-CD19 CDRH2 | VISYEGSNKYYAESVKG |
| 3 | Anti-CD19 CDRH3 | DRGTIFGNYGLEV |
| 4 | Anti-CD19 VH | QVQLVESGGGVVQPGRSLRLSCAASGFTFSSYGMHWVRQAP |
| | | GKCLEWVAVISYEGSNKYYAESVKGRFTISRDNSKNTLYLQM |
| | | NSLRDEDTAVYYCARDRGTIFGNYGLEVWGQGTTVTVSS |
| 5 | Anti-CD19 CDRL1 | RSSQSLLHKNAFNYLD |
| 6 | Anti-CD19 CDRL2 | LGSNRAS |
| 7 | Anti-CD19 CDRL3 | MQALQTPFT |
| 8 | Anti-CD19 VL | DIVMTQSPLSLPVISGEPASISCRSSQSLLHKNAFNYLDWYLQ |
| | | KPGQSPQLLIYLGSNRASGVPDRFSGSGSGTDFTLKISRVEAED |
| | | VGVYYCMQALQTPFTFGCGTKVDIK |
| 9 | Anti-CD19 scFv | DIVMTQSPLSLPVISGEPASISCRSSQSLLHKNAFNYLDWYLQ |
| | | KPGQSPQLLIYLGSNRASGVPDRFSGSGSGTDFTLKISRVEAED |
| | | VGVYYCMQALQTPFTFGCGTKVDIKGGGGSGGGGSGGGGSQ |
| | | VQLVESGGGVVQPGRSLRLSCAASGFTFSSYGMHWVRQAPG |
| | | KCLEWVAVISYEGSNKYYAESVKGRFTISRDNSKNTLYLQMN |
| | | SLRDEDTAVYYCARDRGTIFGNYGLEVWGQGTTVTVSS |
| 10 | CD19 x CD3 scFc | DIVMTQSPLSLPVISGEPASISCRSSQSLLHKNAFNYLDWYLQ |
| | | KPGQSPQLLIYLGSNRASGVPDRFSGSGSGTDFTLKISRVEAED |
| | | VGVYYCMQALQTPFTFGCGTKVDIKGGGGSGGGGSGGGGSQ |
| | | VQLVESGGGVVQPGRSLRLSCAASGFTFSSYGMHWVRQAPG |
| | | KCLEWVAVISYEGSNKYYAESVKGRFTISRDNSKNTLYLQMN |
| | | SLRDEDTAVYYCARDRGTIFGNYGLEVWGQGTTVTVSSGGG |
| | | GSEVLVESGGGLVQPGGSLKLSCAASGFTFNKYAMNWVRQ |
| | | APGKGLEWVARIRSKYNNYATYYADSVKDRFTISRDDSKNT |
| | | AYLQMNNLKTEDTAVYYCVRHGNFGNSYISYWAYWGQGTL |
| | | VTVSSGGGGSGGGGSGGGGSQTVVTQEPSLTVSPGGTVTLTC |
| | | GSSTGAVTSGNYPNWVQQKPGQAPRGLIGGTKFLAPGTPARF |
| | | SGSLLGGKAALTLSGVQPEDEAEYYCVLWYSNRWVFGGGTK |
| | | LTVLGGGGDKTHTCPPCPAPELLGGPSVFLFPPKPKDTLMISR |
| | | TPEVTCVVVDVSHEDPEVKFNWYVDGVEVHNAKTKPCEEQY |
| | | GSTYRCVSVLTVLHQDWLNGKEYKCKVSNKALPAPIEKTISK |
| | | AKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGKGGGGSGGGGSGGGGS |
| | | GGGGSGGGGSGGGGSDKTHTCPPCPAPELLGGPSVFLFPPKPK |
| | | DTLMISRTPEVTCVVVDVSHEDPEVKFNWYVDGVEVHNAKT |
| | | KPCEEQYGSTYRCVSVLTVLHQDWLNGKEYKCKVSNKALPA |
| | | PIEKTISKAKGQPREPQVYTLPPSREEMTKNQVSLTCLVKGFY |
| | | PSDIAVEWESNGQPENNYKTTPPVLDSDGSFFLYSKLTVDKSR |
| | | WQQGNVFSCSVMHEALHNHYTQKSLSLSPGK |
| 11 | Anti-CD33 CDRH1 | NYGMN |
| 12 | Anti-CD33 CDRH2 | WINTYTGEPTYADKFQG |
| 13 | Anti-CD33 CDRH3 | WSWSDGYYVYFDY |
| 14 | Anti-CD33 VH | QVQLVQSGAEVKKPGESVKVSCKASGYTFTNYGMNWVKQA |
| | | PGQCLEWMGWINTYTGEPTYADKFQGRVTMTTDTSTSTAYM |
| | | EIRNLGGDDTAVYYCARWSWSDGYYVYFDYWGQGTSVTVS |
| | | S |
| 15 | Anti-CD33 CDRL1 | KSSQSVLDSSTNKNSLA |
| 16 | Anti-CD33 CDRL2 | WASTRES |
| 17 | Anti-CD33 CDRL3 | QQSAHFPIT |
| 18 | Anti-CD33 VL | DIVMTQSPDSLTVSLGERTTINCKSSQSVLDSSTNKNSLAWYQ |
| | | QKPGQPPKLLLSWASTRESGIPDRFSGSGSGTDFTLTIDSPQPE |
| | | DSATYYCQQSAHFPITFGCGTRLEIK |
| 19 | Anti-CD33 scFv | QVQLVQSGAEVKKPGESVKVSCKASGYTFTNYGMNWVKQA |
| | | PGQCLEWMGWINTYTGEPTYADKFQGRVTMTTDTSTSTAYM |
| | | EIRNLGGDDTAVYYCARWSWSDGYYVYFDYWGQGTSVTVS |
| | | SGGGGSGGGGSGGGGSDIVMTQSPDSLTVSLGERTTINCKSSQ |
| | | SVLDSSTNKNSLAWYQQKPGQPPKLLLSWASTRESGIPDRFSG |
| | | SGSGTDFTLTIDSPQPEDSATYYCQQSAHFPITFGCGTRLEIK |
| 20 | CD33 x CD3 scFc | QVQLVQSGAEVKKPGESVKVSCKASGYTFTNYGMNWVKQA |
| | | PGQCLEWMGWINTYTGEPTYADKFQGRVTMTTDTSTSTAYM |
| | | EIRNLGGDDTAVYYCARWSWSDGYYVYFDYWGQGTSVTVS |
| | | SGGGGSGGGGSGGGGSDIVMTQSPDSLTVSLGERTTINCKSSQ |
| | | SVLDSSTNKNSLAWYQQKPGQPPKLLLSWASTRESGIPDRFSG |
| | | SGSGTDFTLTIDSPQPEDSATYYCQQSAHFPITFGCGTRLEIKS |
| | | GGGGSEVQLVESGGGLVQPGGSLKLSCAASGFTFNKYAMNW |
| | | VRQAPGKGLEWVARIRSKYNNYATYYADSVKDRFTISRDDS |
| | | KNTAYLQMNNLKTEDTAVYYCVRHGNFGNSYISYWAYWGQ |
| | | GTLVTVSSGGGGSGGGGSGGGGSQTVVTQEPSLTVSPGGTVT |
| | | LTCGSSTGAVTSGNYPNWVQQKPGQAPRGLIGGTKFLAPGTP |
| | | ARFSGSLLGGKAALTLSGVQPEDEAEYYCVLWYSNRWVFGG |
| | | GTKLTVLGGGGDKTHTCPPCPAPELLGGPSVFLFPPKPKDTLM |
| | | ISRTPEVTCVVVDVSHEDPEVKFNWYVDGVEVHNAKTKPCEE |
| | | QYGSTYRCVSVLTVLHQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGKGGGGSGGGGSGGG |
| | | GSGGGGSGGGGSGGGGSDKTHTCPPCPAPELLGGPSVFLFPPK |
| | | PKDTLMISRTPEVTCVVVDVSHEDPEVKFNWYVDGVEVHNA |
| | | KTKPCEEQYGSTYRCVSVLTVLHQDWLNGKEYKCKVSNKAL |
| | | PAPIEKTISKAKGQPREPQVYTLPPSREEMTKNQVSLTCLVKG |
| | | FYPSDIAVEWESNGQPENNYKTTPPVLDSDGSFFLYSKLTVDK |
| | | SRWQQGNVFSCSVMHEALHNHYTQKSLSLSPGK |
| 21 | Anti-FLT3 CDRH1 | NARMGVS |
| 22 | Anti-FLT3 CDRH2 | HIFSNDEKSYSTSLKN |
| 23 | Anti-FLT3 CDRH3 | IVGYGSGWYGFFDY |
| 24 | Anti-FLT3 VH | QVTLKESGPTLVKPTETLTLTCTLSGFSLNNARMGVSWIRQPP |
| | | GKCLEWLAHIFSNDEKSYSTSLKNRLTISKDSSKTQVVLTMTN |
| | | VDPVDTATYYCARIVGYGSGWYGFFDYWGQGTLVTVSS |
| 25 | Anti-FLT3 CDRL1 | RASQGIRNDLG |
| 26 | Anti-FLT3 CDRL2 | AASTLQS |
| 27 | Anti-FLT3 CDRL3 | LQHNSYPLT |
| 28 | Anti-FLT3 VL | DIQMTQSPSSLSASVGDRVTITCRASQGIRNDLGWYQQKPGK |
| | | APKRLIYAASTLQSGVPSRFSGSGSGTEFTLTISSLQPEDFATY |
| | | YCLQHNSYPLTFGCGTKVEIK |
| 29 | Anti-FLT3 scFv | QVTLKESGPTLVKPTETLTLTCTLSGFSLNNARMGVSWIRQPP |
| | | GKCLEWLAHIFSNDEKSYSTSLKNRLTISKDSSKTQVVLTMTN |
| | | VDPVDTATYYCARIVGYGSGWYGFFDYWGQGTLVTVSSGGG |
| | | GSGGGGSGGGGSDIQMTQSPSSLSASVGDRVTITCRASQGIRN |
| | | DLGWYQQKPGKAPKRLIYAASTLQSGVPSRFSGSGSGTEFTLT |
| | | ISSLQPEDFATYYCLQHNSYPLTFGCGTKVEIK |
| 30 | FLT3 x CD3 scFc | QVTLKESGPTLVKPTETLTLTCTLSGFSLNNARMGVSWIRQPP |
| | | GKCLEWLAHIFSNDEKSYSTSLKNRLTISKDSSKTQVVLTMTN |
| | | VDPVDTATYYCARIVGYGSGWYGFFDYWGQGTLVTVSSGGG |
| | | GSGGGGSGGGGSDIQMTQSPSSLSASVGDRVTITCRASQGIRN |
| | | DLGWYQQKPGKAPKRLIYAASTLQSGVPSRFSGSGSGTEFTLT |
| | | ISSLQPEDFATYYCLQHNSYPLTFGCGTKVEIKSGGGGSEVQL |
| | | VESGGGLVQPGGSLKLSCAASGFTFNKYAMNWVRQAPGKGL |
| | | EWVARIRSKYNNYATYYADSVKDRFTISRDDSKNTAYLQMN |
| | | NLKTEDTAVYYCVRHGNFGNSYISYWAYWGQGTLVTVSSGG |
| | | GGSGGGGSGGGGSQTVVTQEPSLTVSPGGTVTLTCGSSTGAV |
| | | TSGNYPNWVQQKPGQAPRGLIGGTKFLAPGTPARFSGSLLGG |
| | | KAALTLSGVQPEDEAEYYCVLWYSNRWVFGGGTKLTVLGG |
| | | GGDKTHTCPPCPAPELLGGPSVFLFPPKPKDTLMISRTPEVTCV |
| | | VVDVSHEDPEVKFNWYVDGVEVHNAKTKPCEEQYGSTYRC |
| | | VSVLTVLHQDWLNGKEYKCKVSNKALPAPIEKTISKAKGQPR |
| | | EPQVYTLPPSREEMTKNQVSLTCLVKGFYPSDIAVEWESNGQ |
| | | PENNYKTTPPVLDSDGSFFLYSKLTVDKSRWQQGNVFSCSVM |
| | | HEALHNHYTQKSLSLSPGKGGGGSGGGGSGGGGSGGGGSGG |
| | | GGSGGGGSDKTHTCPPCPAPELLGGPSVFLFPPKPKDTLMISR |
| | | TPEVTCVVVDVSHEDPEVKFNWYVDGVEVHNAKTKPCEEQY |
| | | GSTYRCVSVLTVLHQDWLNGKEYKCKVSNKALPAPIEKTISK |
| | | AKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 31 | Anti-DLL3 CDRH1 | SYYWS |
| 32 | Anti-DLL3 CDRH2 | YVYYSGTTNYNPSLKS |
| 33 | Anti-DLL3 CDRH3 | IAVTGFYFDY |
| 34 | Anti-DLL3 VH | QVQLQESGPGLVKPSETLSLTCTVSGGSISSYYWSWIRQPPGK |
| | | CLEWIGYVYYSGTTNYNPSLKSRVTISVDTSKNQFSLKLSSVT |
| | | AADTAVYYCASIAVTGFYFDYWGQGTLVTVSS |
| 35 | Anti-DLL3 CDRL1 | RASQRVNNNYLA |
| 36 | Anti-DLL3 CDRL2 | GASSRAT |
| 37 | Anti-DLL3 CDRL3 | QQYDRSPLT |
| 38 | Anti-DLL3 VL | EIVLTQSPGTLSLSPGERVTLSCRASQRVNNNYLAWYQQRPG |
| | | QAPRLLIYGASSRATGIPDRFSGSGSGTDFTLTISRLEPEDFAV |
| | | YYCQQYDRSPLTFGCGTKLEIK |
| 39 | Anti-DLL3 scFv | QVQLQESGPGLVKPSETLSLTCTVSGGSISSYYWSWIRQPPGK |
| | | CLEWIGYVYYSGTTNYNPSLKSRVTISVDTSKNQFSLKLSSVT |
| | | AADTAVYYCASIAVTGFYFDYWGQGTLVTVSSGGGGSGGGG |
| | | SGGGGSEIVLTQSPGTLSLSPGERVTLSCRASQRVNNNYLAW |
| | | YQQRPGQAPRLLIYGASSRATGIPDRFSGSGSGTDFTLTISRLE |
| | | PEDFAVYYCQQYDRSPLTFGCGTKLEIK |
| 40 | DLL3 x CD3 scFc | QVQLQESGPGLVKPSETLSLTCTVSGGSISSYYWSWIRQPPGK |
| | | CLEWIGYVYYSGTTNYNPSLKSRVTISVDTSKNQFSLKLSSVT |
| | | AADTAVYYCASIAVTGFYFDYWGQGTLVTVSSGGGGSGGGG |
| | | SGGGGSEIVLTQSPGTLSLSPGERVTLSCRASQRVNNNYLAW |
| | | YQQRPGQAPRLLIYGASSRATGIPDRFSGSGSGTDFTLTISRLE |
| | | PEDFAVYYCQQYDRSPLTFGCGTKLEIKSGGGGSEVQLVESG |
| | | GGLVQPGGSLKLSCAASGFTFNKYAMNWVRQAPGKGLEWV |
| | | ARIRSKYNNYATYYADSVKDRFTISRDDSKNTAYLQMNNLK |
| | | TEDTAVYYCVRHGNFGNSYISYWAYWGQGTLVTVSSGGGGS |
| | | GGGGSGGGGSQTVVTQEPSLTVSPGGTVTLTCGSSTGAVTSG |
| | | NYPNWVQQKPGQAPRGLIGGTKFLAPGTPARFSGSLLGGKAA |
| | | LTLSGVQPEDEAEYYCVLWYSNRWVFGGGTKLTVLGGGGD |
| | | KTHTCPPCPAPELLGGPSVFLFPPKPKDTLMISRTPEVTCVVVD |
| | | VSHEDPEVKFNWYVDGVEVHNAKTKPCEEQYGSTYRCVSVL |
| | | TVLHQDWLNGKEYKCKVSNKALPAPIEKTISKAKGQPREPQV |
| | | YTLPPSREEMTKNQVSLTCLVKGFYPSDIAVEWESNGQPENN |
| | | YKTTPPVLDSDGSFFLYSKLTVDKSRWQQGNVFSCSVMHEAL |
| | | HNHYTQKSLSLSPGGGGSGGGGSGGGGSGGGGSGGGGSGGG |
| | | GSDKTHTCPPCPAPELLGGPSVFLFPPKPKDTLMISRTPEVTCV |
| | | VVDVSHEDPEVKFNWYVDGVEVHNAKTKPCEEQYGSTYRC |
| | | VSVLTVLHQDWLNGKEYKCKVSNKALPAPIEKTISKAKGQPR |
| | | EPQVYTLPPSREEMTKNQVSLTCLVKGFYPSDIAVEWESNGQ |
| | | PENNYKTTPPVLDSDGSFFLYSKLTVDKSRWQQGNVFSCSVM |
| | | HEALHNHYTQKSLSLSPGK |
| 41 | Anti-BCMA | NHIIH |
| | CDRH1 | |
| 42 | Anti-BCMA | YINPYPGYHAYNEKFQG |
| | CDRH2 | |
| 43 | Anti-BCMA | DGYYRDTDVLDY |
| | CDRH3 | |
| 44 | Anti-BCMA VH | QVQLVQSGAEVKKPGASVKVSCKASGYTFTNHIIHWVRQAP |
| | | GQCLEWMGYINPYPGYHAYNEKFQGRATMTSDTSTSTVYME |
| | | LSSLRSEDTAVYYCARDGYYRDTDVLDYWGQGTLVTVSS |
| 45 | Anti-BCMA | QASQDISNYLN |
| | CDRLI | |
| 46 | Anti-BCMA | YTSRLHT |
| | CDRL2 | |
| 47 | Anti-BCMA | QQGNTLPWT |
| | CDRL3 | |
| 48 | Anti-BCMA VL | DIQMTQSPSSLSASVGDRVTITCQASQDISNYLNWYQQKPGK |
| | | APKLLIYYTSRLHTGVPSRFSGSGSGTDFTFTISSLEPEDIATYY |
| | | CQQGNTLPWTFGCGTKVEIK |
| 49 | Anti-BCMA scFv | QVQLVQSGAEVKKPGASVKVSCKASGYTFTNHIIHWVRQAP |
| | | GQCLEWMGYINPYPGYHAYNEKFQGRATMTSDTSTSTVYME |
| | | LSSLRSEDTAVYYCARDGYYRDTDVLDYWGQGTLVTVSSGG |
| | | GGSGGGGSGGGGSDIQMTQSPSSLSASVGDRVTITCQASQDIS |
| | | NYLNWYQQKPGKAPKLLIYYTSRLHTGVPSRFSGSGSGTDFT |
| | | FTISSLEPEDIATYYCQQGNTLPWTFGCGTKVEIK |
| 50 | BCMA x CD3 scFc | QVQLVQSGAEVKKPGASVKVSCKASGYTFTNHIIHWVRQAP |
| | | GQCLEWMGYINPYPGYHAYNEKFQGRATMTSDTSTSTVYME |
| | | LSSLRSEDTAVYYCARDGYYRDTDVLDYWGQGTLVTVSSGG |
| | | GGSGGGGSGGGGSDIQMTQSPSSLSASVGDRVTITCQASQDIS |
| | | NYLNWYQQKPGKAPKLLIYYTSRLHTGVPSRFSGSGSGTDFT |
| | | FTISSLEPEDIATYYCQQGNTLPWTFGCGTKVEIKSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEV |
| | | TCVVVDVSHEDPEVKFNWYVDGVEVHNAKTKPCEEQYGSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGKGGGGSGGGGSGGGGSGGGGS |
| | | GGGGSGGGGSDKTHTCPPCPAPELLGGPSVFLFPPKPKDTLMI |
| | | SRTPEVTCVVVDVSHEDPEVKFNWYVDGVEVHNAKTKPCEE |
| | | QYGSTYRCVSVLTVLHQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| 51 | Anti-PSMA CDRH1 | DYYMY |
| 52 | Anti-PSMA CDRH2 | IISDGGYYTYYSDIIKG |
| 53 | Anti-PSMA CDRH3 | GFPLLRHGAMDY |
| 54 | Anti-PSMA VH | QVQLVESGGGLVKPGESLRLSCAASGFTFSDYYMYWVRQAP |
| | | GKCLEWVAIISDGGYYTYYSDIIKGRFTISRDNAKNSLYLQMN |
| | | SLKAEDTAVYYCARGFPLLRHGAMDYWGQGTLVTVSS |
| 55 | Anti-PSMA CDRL1 | KASQNVDTNVA |
| 56 | Anti-PSMA CDRL2 | SASYVYW |
| 57 | Anti-PSMA CDRL3 | QQYDQQLIT |
| 58 | Anti-PSMA VL | DIQMTQSPSSLSASVGDRVTITCKASQNVDTNVAWYQQKPGQ |
| | | APKSLIYSASYVYWDVPSRFSGSASGTDFTLTISSVQSEDFATY |
| | | YCQQYDQQLITFGCGTKLEIK |
| 59 | Anti-PSMA scFv | QVQLVESGGGLVKPGESLRLSCAASGFTFSDYYMYWVRQAP |
| | | GKCLEWVAIISDGGYYTYYSDIIKGRFTISRDNAKNSLYLQMN |
| | | SLKAEDTAVYYCARGFPLLRHGAMDYWGQGTLVTVSSGGG |
| | | GSGGGGSGGGGSDIQMTQSPSSLSASVGDRVTITCKASQNVD |
| | | TNVAWYQQKPGQAPKSLIYSASYVYWDVPSRFSGSASGTDFT |
| | | LTISSVQSEDFATYYCQQYDQQLITFGCGTKLEIK |
| 60 | PSMA x CD3 scFc | QVQLVESGGGLVKPGESLRLSCAASGFTFSDYYMYWVRQAP |
| | | GKCLEWVAIISDGGYYTYYSDIIKGRFTISRDNAKNSLYLQMN |
| | | SLKAEDTAVYYCARGFPLLRHGAMDYWGQGTLVTVSSGGG |
| | | GSGGGGSGGGGSDIQMTQSPSSLSASVGDRVTITCKASQNVD |
| | | TNVAWYQQKPGQAPKSLIYSASYVYWDVPSRFSGSASGTDFT |
| | | LTISSVQSEDFATYYCQQYDQQLITFGCGTKLEIKSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQ |
| | | MNNLKTEDTAVYYCVRHGNFGNSYISYWAYWGQGTLVTVS |
| | | SGGGGSGGGGSGGGGSQTVVTQEPSLTVSPGGTVTLTCGSST |
| | | GAVTSGNYPNWVQQKPGQAPRGLIGGTKFLAPGTPARFSGSL |
| | | LGGKAALTLSGVQPEDEAEYYCVLWYSNRWVFGGGTKLTVL |
| | | GGGGDKTHTCPPCPAPELLGGPSVFLFPPKPKDTLMISRTPEV |
| | | TCVVVDVSHEDPEVKFNWYVDGVEVHNAKTKPCEEQYGSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGKGGGGSGGGGSGGGGSGGGGS |
| | | GGGGSGGGGSDKTHTCPPCPAPELLGGPSVFLFPPKPKDTLMI |
| | | SRTPEVTCVVVDVSHEDPEVKFNWYVDGVEVHNAKTKPCEE |
| | | QYGSTYRCVSVLTVLHQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| 61 | Extracellular | QDGNEEMGGITQTPYKVSISGTTVILTCPQYPGSEILWQHNDK |
| | domain of human | NIGGDEDDKNIGSDEDHLSLKEFSELEQSGYYVCYPRGSKPED |
| | CD3 epsilon | ANFYLYLRARVCENCMEMD |
| 62 | Anti-CD3 F6A | IYAMN |
| | CDRH1 | |
| 63 | Anti-CD3 F6A | RIRSKYNNYATYYADSVKS |
| | CDRH2 | |
| 64 | Anti-CD3 F6A | HGNFGNSYVSFFAY |
| | CDRH3 | |
| 65 | Anti-CD3 H2C | KYAMN |
| | CDRH1 | |
| 66 | Anti-CD3 H2C | RIRSKYNNYATYYADSVKD |
| | CDRH2 | |
| 67 | Anti-CD3 H2C | HGNFGNSYISYWAY |
| | CDRH3 | |
| 68 | Anti-CD3 H1E | SYAMN |
| | CDRH1 | |
| 69 | Anti-CD3 H1E | RIRSKYNNYATYYADSVKG |
| | CDRH2 | |
| 70 | Anti-CD3 H1E | HGNFGNSYLSFWAY |
| | CDRH3 | |
| 71 | Anti-CD3 G4H | RYAMN |
| | CDRH1 | |
| 69 | Anti-CD3 G4H | RIRSKYNNYATYYADSVKG |
| | CDRH2 | |
| 72 | Anti-CD3 G4H | HGNFGNSYLSYFAY |
| | CDRH3 | |
| 65 | Anti-CD3 E1L | KYAMN |
| | CDRH1 | |
| 63 | Anti-CD3 E1L | RIRSKYNNYATYYADSVKS |
| | CDRH2 | |
| 73 | Anti-CD3 E1L | HGNFGNSYTSYYAY |
| | CDRH3 | |
| 74 | Anti-CD3 F7O | VYAMN |
| | CDRH1 | |
| 75 | Anti-CD3 F7O | RIRSKYNNYATYYADSVKK |
| | CDRH2 | |
| 76 | Anti-CD3 F7O | HGNFGNSYISWWAY |
| | CDRH3 | |
| 74 | Anti-CD3 A2J | VYAMN |
| | CDRH1 | |
| 75 | Anti-CD3 A2J | RIRSKYNNYATYYADSVKK |
| | CDRH2 | |
| 77 | Anti-CD3 A2J | HGNFGNSYLSWWAY |
| | CDRH3 | |
| 78 | Anti-CD3 E2M | GYAMN |
| | CDRH1 | |
| 79 | Anti-CD3 E2M | RIRSKYNNYATYYADSVKE |
| | CDRH2 | |
| 80 | Anti-CD3 E2M | HRNFGNSYLSWFAY |
| | CDRH3 | |
| 68 | Anti-CD3 F12Q | SYAMN |
| | CDRH1 | |
| 69 | Anti-CD3 F12Q | RIRSKYNNYATYYADSVKG |
| | CDRH2 | |
| 81 | Anti-CD3 F12Q | HGNFGNSYVSWWAY |
| | CDRH3 | |
| 65 | Anti-CD3 I2C | KYAMN |
| | CDRH1 | |
| 66 | Anti-CD3 I2C | RIRSKYNNYATYYADSVKD |
| | CDRH2 | |
| 67 | Anti-CD3 I2C | HGNFGNSYISYWAY |
| | CDRH3 | |
| 82 | Anti-CD3 F6A | GSSTGAVTSGYYPN |
| | CDRL1 | |
| 83 | Anti-CD3 F6A | GTKFLAP |
| | CDRL2 | |
| 84 | Anti-CD3 F6A | ALWYSNRWV |
| | CDRL3 | |
| 82 | Anti-CD3 H2C | GSSTGAVTSGYYPN |
| | CDRL1 | |
| 83 | Anti-CD3 H2C | GTKFLAP |
| | CDRL2 | |
| 84 | Anti-CD3 H2C | ALWYSNRWV |
| | CDRL3 | |
| 82 | Anti-CD3 H1E | GSSTGAVTSGYYPN |
| | CDRL1 | |
| 83 | Anti-CD3 H1E | GTKFLAP |
| | CDRL2 | |
| 84 | Anti-CD3 H1E | ALWYSNRWV |
| | CDRL3 | |
| 82 | Anti-CD3 G4H | GSSTGAVTSGYYPN |
| | CDRL1 | |
| 83 | Anti-CD3 G4H | GTKFLAP |
| | CDRL2 | |
| 84 | Anti-CD3 G4H | ALWYSNRWV |
| | CDRL3 | |
| 82 | Anti-CD3 E1L | GSSTGAVTSGYYPN |
| | CDRL1 | |
| 83 | Anti-CD3 E1L | GTKFLAP |
| | CDRL2 | |
| 84 | Anti-CD3 E1L | ALWYSNRWV |
| | CDRL3 | |
| 82 | Anti-CD3 F7O | GSSTGAVTSGYYPN |
| | CDRLI | |
| 83 | Anti-CD3 F7O | GTKFLAP |
| | CDRL2 | |
| 84 | Anti-CD3 F7O | ALWYSNRWV |
| | CDRL3 | |
| 85 | Anti-CD3 A2J | RSSTGAVTSGYYPN |
| | CDRL1 | |
| 86 | Anti-CD3 A2J | ATDMRPS |
| | CDRL2 | |
| 84 | Anti-CD3 A2J | ALWYSNRWV |
| | CDRL3 | |
| 85 | Anti-CD3 E2M | RSSTGAVTSGYYPN |
| | CDRL1 | |
| 86 | Anti-CD3 E2M | ATDMRPS |
| | CDRL2 | |
| 84 | Anti-CD3 E2M | ALWYSNRWV |
| | CDRL3 | |
| 87 | Anti-CD3 F12Q | GSSTGAVTSGNYPN |
| | CDRL1 | |
| 83 | Anti-CD3 F12Q | GTKFLAP |
| | CDRL2 | |
| 88 | Anti-CD3 F12Q | VLWYSNRWV |
| | CDRL3 | |
| 87 | Anti-CD3 I2C | GSSTGAVTSGNYPN |
| | CDRL1 | |
| 83 | Anti-CD3 I2C | GTKFLAP |
| | CDRL2 | |
| 88 | Anti-CD3 I2C | VLWYSNRWV |
| | CDRL3 | |
| 89 | Anti-CD3 F6A VH | EVQLVESGGGLVQPGGSLKLSCAASGFTFNIYAMNWVRQAP |
| | | GKGLEWVARIRSKYNNYATYYADSVKSRFTISRDDSKNTAYL |
| | | QMNNLKTEDTAVYYCVRHGNFGNSYVSFFAYWGQGTLVTV |
| | | SS |
| 90 | Anti-CD3 H2C VH | EVQLVESGGGLVQPGGSLKLSCAASGFTFNKYAMNWVRQAP |
| | | GKGLEWVARIRSKYNNYATYYADSVKDRFTISRDDSKNTAY |
| | | LQMNNLKTEDTAVYYCVRHGNFGNSYISYWAYWGQGTLVT |
| | | VSS |
| 91 | Anti-CD3 H1E VH | EVQLVESGGGLEQPGGSLKLSCAASGFTFNSYAMNWVRQAP |
| | | GKGLEWVARIRSKYNNYATYYADSVKGRFTISRDDSKNTAY |
| | | LQMNNLKTEDTAVYYCVRHGNFGNSYLSFWAYWGQGTLVT |
| | | VSS |
| 92 | Anti-CD3 G4H VH | EVQLVESGGGLVQPGGSLKLSCAASGFTFNRYAMNWVRQAP |
| | | GKGLEWVARIRSKYNNYATYYADSVKGRFTISRDDSKNTAY |
| | | LQMNNLKTEDTAVYYCVRHGNFGNSYLSYFAYWGQGTLVT |
| | | VSS |
| 93 | Anti-CD3 E1L VH | EVQLVESGGGLVQPGGSLKLSCAASGFTFNKYAMNWVRQAP |
| | | GKGLEWVARIRSKYNNYATYYADSVKSRFTISRDDSKNTAYL |
| | | QMNNLKTEDTAVYYCVRHGNFGNSYTSYYAYWGQGTLVTV |
| | | SS |
| 94 | Anti-CD3 F7O VH | EVQLVESGGGLVQPGGSLKLSCAASGFTFNVYAMNWVRQAP |
| | | GKGLEWVARIRSKYNNYATYYADSVKKRFTISRDDSKNTAY |
| | | LQMNNLKTEDTAVYYCVRHGNFGNSYISWWAYWGQGTLVT |
| | | VSS |
| 95 | Anti-CD3 A2J VH | EVQLVESGGGLVQPGGSLKLSCAASGFTFNVYAMNWVRQAP |
| | | GKGLEWVARIRSKYNNYATYYADSVKKRFTISRDDSKNTAY |
| | | LQMNNLKTEDTAVYYCVRHGNFGNSYLSWWAYWGQGTLV |
| | | TVSS |
| 96 | Anti-CD3 E2M VH | EVQLVESGGGLVQPGGSLKLSCAASGFTFNGYAMNWVRQAP |
| | | GKGLEWVARIRSKYNNYATYYADSVKERFTISRDDSKNTAYL |
| | | QMNNLKTEDTAVYYCVRHRNFGNSYLSWFAYWGQGTLVTV |
| | | SS |
| 97 | Anti-CD3 F12Q VH | EVQLVESGGGLVQPGGSLKLSCAASGFTFNSYAMNWVRQAP |
| | | GKGLEWVARIRSKYNNYATYYADSVKGRFTISRDDSKNTAY |
| | | LQMNNLKTEDTAVYYCVRHGNFGNSYVSWWAYWGQGTLV |
| | | TVSS |
| 90 | Anti-CD3 I2C VH | EVQLVESGGGLVQPGGSLKLSCAASGFTFNKYAMNWVRQAP |
| | | GKGLEWVARIRSKYNNYATYYADSVKDRFTISRDDSKNTAY |
| | | LQMNNLKTEDTAVYYCVRHGNFGNSYISYWAYWGQGTLVT |
| | | VSS |
| 98 | Anti-CD3 F6A VL | QTVVTQEPSLTVSPGGTVTLTCGSSTGAVTSGYYPNWVQQKP |
| | | GQAPRGLIGGTKFLAPGTPARFSGSLLGGKAALTLSGVQPEDE |
| | | AEYYCALWYSNRWVFGGGTKLTVL |
| 98 | Anti-CD3 H2C VL | QTVVTQEPSLTVSPGGTVTLTCGSSTGAVTSGYYPNWVQQKP |
| | | GQAPRGLIGGTKFLAPGTPARFSGSLLGGKAALTLSGVQPEDE |
| | | AEYYCALWYSNRWVFGGGTKLTVL |
| 98 | Anti-CD3 H1E VL | QTVVTQEPSLTVSPGGTVTLTCGSSTGAVTSGYYPNWVQQKP |
| | | GQAPRGLIGGTKFLAPGTPARFSGSLLGGKAALTLSGVQPEDE |
| | | AEYYCALWYSNRWVFGGGTKLTVL |
| 98 | Anti-CD3 G4H VL | QTVVTQEPSLTVSPGGTVTLTCGSSTGAVTSGYYPNWVQQKP |
| | | GQAPRGLIGGTKFLAPGTPARFSGSLLGGKAALTLSGVQPEDE |
| | | AEYYCALWYSNRWVFGGGTKLTVL |
| 98 | Anti-CD3 E1L VL | QTVVTQEPSLTVSPGGTVTLTCGSSTGAVTSGYYPNWVQQKP |
| | | GQAPRGLIGGTKFLAPGTPARFSGSLLGGKAALTLSGVQPEDE |
| | | AEYYCALWYSNRWVFGGGTKLTVL |
| 98 | Anti-CD3 F7O VL | QTVVTQEPSLTVSPGGTVTLTCGSSTGAVTSGYYPNWVQQKP |
| | | GQAPRGLIGGTKFLAPGTPARFSGSLLGGKAALTLSGVQPEDE |
| | | AEYYCALWYSNRWVFGGGTKLTVL |
| 99 | Anti-CD3 A2J VL | QTVVTQEPSLTVSPGGTVTLTCRSSTGAVTSGYYPNWVQQKP |
| | | GQAPRGLIGATDMRPSGTPARFSGSLLGGKAALTLSGVQPED |
| | | EAEYYCALWYSNRWVFGGGTKLTVL |
| 99 | Anti-CD3 E2M VL | QTVVTQEPSLTVSPGGTVTLTCRSSTGAVTSGYYPNWVQQKP |
| | | GQAPRGLIGATDMRPSGTPARFSGSLLGGKAALTLSGVQPED |
| | | EAEYYCALWYSNRWVFGGGTKLTVL |
| 100 | Anti-CD3 F12Q VL | QTVVTQEPSLTVSPGGTVTLTCGSSTGAVTSGNYPNWVQQKP |
| | | GQAPRGLIGGTKFLAPGTPARFSGSLLGGKAALTLSGVQPEDE |
| | | AEYYCVLWYSNRWVFGGGTKLTVL |
| 100 | Anti-CD3 I2C VL | QTVVTQEPSLTVSPGGTVTLTCGSSTGAVTSGNYPNWVQQKP |
| | | GQAPRGLIGGTKFLAPGTPARFSGSLLGGKAALTLSGVQPEDE |
| | | AEYYCVLWYSNRWVFGGGTKLTVL |
| 101 | Anti-CD3 F6A scFv | EVLVESGGGLVQPGGSLKLSCAASGFTFNIYAMNWVRQAP |
| | | GKGLEWVARIRSKYNNYATYYADSVKSRFTISRDDSKNTAYL |
| | | QMNNLKTEDTAVYYCVRHGNFGNSYVSFFAYWGQGTLVTV |
| | | SSGGGGSGGGGSGGGGSQTVVTQEPSLTVSPGGTVTLTCGSS |
| | | TGAVTSGYYPNWVQQKPGQAPRGLIGGTKFLAPGTPARFSGS |
| | | LLGGKAALTLSGVQPEDEAEYYCALWYSNRWVFGGGTKLTV |
| | | L |
| 102 | Anti-CD3 H2C scFv | EVQLVESGGGLVQPGGSLKLSCAASGFTFNKYAMNWVRQAP |
| | | GKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGYYPNWVQQKPGQAPRGLIGGTKFLAPGTPARFSG |
| | | SLLGGKAALTLSGVQPEDEAEYYCALWYSNRWVFGGGTKLT |
| | | VL |
| 103 | Anti-CD3 H1E scFv | EVQLVESGGGLEQPGGSLKLSCAASGFTFNSYAMNWVRQAP |
| | | GKGLEWVARIRSKYNNYATYYADSVKGRFTISRDDSKNTAY |
| | | LQMNNLKTEDTAVYYCVRHGNFGNSYLSFWAYWGQGTLVT |
| | | VSSGGGGSGGGGSGGGGSQTVVTQEPSLTVSPGGTVTLTCGS |
| | | STGAVTSGYYPNWVQQKPGQAPRGLIGGTKFLAPGTPARFSG |
| | | SLLGGKAALTLSGVQPEDEAEYYCALWYSNRWVFGGGTKLT |
| | | VL |
| 104 | Anti-CD3 G4H scFv | EVQLVESGGGLVQPGGSLKLSCAASGFTFNRYAMNWVRQAP |
| | | GKGLEWVARIRSKYNNYATYYADSVKGRFTISRDDSKNTAY |
| | | LQMNNLKTEDTAVYYCVRHGNFGNSYLSYFAYWGQGTLVT |
| | | VSSGGGGSGGGGSGGGGSQTVVTQEPSLTVSPGGTVTLTCGS |
| | | STGAVTSGYYPNWVQQKPGQAPRGLIGGTKFLAPGTPARFSG |
| | | SLLGGKAALTLSGVQPEDEAEYYCALWYSNRWVFGGGTKLT |
| | | VL |
| 105 | Anti-CD3 E1L scFv | EVQLVESGGGLVQPGGSLKLSCAASGFTFNKYAMNWVRQAP |
| | | GKGLEWVARIRSKYNNYATYYADSVKSRFTISRDDSKNTAYL |
| | | QMNNLKTEDTAVYYCVRHGNFGNSYTSYYAYWGQGTLVTV |
| | | SSGGGGSGGGGSGGGGSQTVVTQEPSLTVSPGGTVTLTCGSS |
| | | TGAVTSGYYPNWVQQKPGQAPRGLIGGTKFLAPGTPARFSGS |
| | | LLGGKAALTLSGVQPEDEAEYYCALWYSNRWVFGGGTKLTV |
| | | L |
| 106 | Anti-CD3 F7O scFv | EVQLVESGGGLVQPGGSLKLSCAASGFTFNVYAMNWVRQAP |
| | | GKGLEWVARIRSKYNNYATYYADSVKKRFTISRDDSKNTAY |
| | | LQMNNLKTEDTAVYYCVRHGNFGNSYISWWAYWGQGTLVT |
| | | VSSGGGGSGGGGSGGGGSQTVVTQEPSLTVSPGGTVTLTCGS |
| | | STGAVTSGYYPNWVQQKPGQAPRGLIGGTKFLAPGTPARFSG |
| | | SLLGGKAALTLSGVQPEDEAEYYCALWYSNRWVFGGGTKLT |
| | | VL |
| 107 | Anti-CD3 A2J scFv | EVQLVESGGGLVQPGGSLKLSCAASGFTFNVYAMNWVRQAP |
| | | GKGLEWVARIRSKYNNYATYYADSVKKRFTISRDDSKNTAY |
| | | LQMNNLKTEDTAVYYCVRHGNFGNSYLSWWAYWGQGTLV |
| | | TVSSGGGGSGGGGSGGGGSQTVVTQEPSLTVSPGGTVTLTCR |
| | | SSTGAVTSGYYPNWVQQKPGQAPRGLIGATDMRPSGTPARFS |
| | | GSLLGGKAALTLSGVQPEDEAEYYCALWYSNRWVFGGGTKL |
| | | TVL |
| 108 | Anti-CD3 E2M | EVQLVESGGGLVQPGGSLKLSCAASGFTFNGYAMNWVRQAP |
| | scFv | GKGLEWVARIRSKYNNYATYYADSVKERFTISRDDSKNTAYL |
| | | QMNNLKTEDTAVYYCVRHRNFGNSYLSWFAYWGQGTLVTV |
| | | SSGGGGSGGGGSGGGGSQTVVTQEPSLTVSPGGTVTLTCRSS |
| | | TGAVTSGYYPNWVQQKPGQAPRGLIGATDMRPSGTPARFSGS |
| | | LLGGKAALTLSGVQPEDEAEYYCALWYSNRWVFGGGTKLTV |
| | | L |
| 109 | Anti-CD3 F12Q | EVQLVESGGGLVQPGGSLKLSCAASGFTFNSYAMNWVRQAP |
| | scFv | GKGLEWVARIRSKYNNYATYYADSVKGRFTISRDDSKNTAY |
| | | LQMNNLKTEDTAVYYCVRHGNFGNSYVSWWAYWGQGTLV |
| | | TVSSGGGGSGGGGSGGGGSQTVVTQEPSLTVSPGGTVTLTCG |
| | | SSTGAVTSGNYPNWVQQKPGQAPRGLIGGTKFLAPGTPARFS |
| | | GSLLGGKAALTLSGVQPEDEAEYYCVLWYSNRWVFGGGTKL |
| | | TVL |
| 110 | Anti-CD3 I2C scFv | EVQLVESGGGLVQPGGSLKLSCAASGFTFNKYAMNWVRQAP |
| | | GKGLEWVARIRSKYNNYATYYADSVKDRFTISRDDSKNTAY |
| | | LQMNNLKTEDTAVYYCVRHGNFGNSYISYWAYWGQGTLVT |
| | | VSSGGGGSGGGGSGGGGSQTVVTQEPSLTVSPGGTVTLTCGS |
| | | STGAVTSGNYPNWVQQKPGQAPRGLIGGTKFLAPGTPARFSG |
| | | SLLGGKAALTLSGVQPEDEAEYYCVLWYSNRWVFGGGTKLT |
| | | VL |
| 111 | Linker 1 | GGGG |
| 112 | Linker 2 | GGGGS |
| 113 | Linker 3 | PGGGGS |
| 114 | Linker 4 | PGGDGS |
| 115 | Linker 5 | SGGGGS |
| 116 | Linker 6 | GGGGSGGGS |
| 117 | Linker 7 | GGGGQ |
| 118 | (G₄S)₂ linker | GGGGSGGGGS |
| 119 | (G₄S)₃ linker | GGGGSGGGGSGGGGS |
| 120 | (G₄S)₄ linker | GGGGSGGGGSGGGGSGGGGS |
| 121 | (G₄S)₅ linker | GGGGSGGGGSGGGGSGGGGSGGGGS |
| 122 | (G₄S)₆ linker | GGGGSGGGGSGGGGSGGGGSGGGGSGGGGS |
| 123 | (G₄S)₇ linker | GGGGSGGGGSGGGGSGGGGSGGGGSGGGGSGGGGS |
| 124 | (G₄S)₈ linker | GGGGSGGGGSGGGGSGGGGSGGGGSGGGGSGGGGSGGGGS |
| 125 | IgG1 hinge-1 | DKTHTCPPCP |
| 126 | IgG1 hinge-2 | EPKSCDKTHTCPPCP |
| 127 | IgG2 hinge | ERKCCVECPPCP |
| 128 | IgG3 hinge-1 | ELKTPLDTTHTCPRCP |
| 129 | IgG3 hinge-2 | EPKSCDTPPPCPRCP |
| 130 | IgG3 hinge-3 | ELKTPLGDTTHTCPRCP |
| 131 | IgG4 hinge | ESKYGPPCPSCP |
| 132 | Fc monomer-1 | DKTHTCPPCPAPELLGGPSVFLFPPKPKDTLMISRTPEVTCVVV |
| | | DVSHEDPEVKFNWYVDGVEVHNAKTKPCEEQYGSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGK |
| 133 | Fc monomer-2 | DKTHTCPPCPAPELLGGPSVFLFPPKPKDTLMISRTPEVTCVVV |
| | | DVSHEDPEVKFNWYVDGVEVHNAKTKPCEEQYGSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSP |
| 134 | Fc monomer-3 | DKTHTCPPCPAPELLGGPSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGK |
| 135 | Fc monomer-4 | DKTHTCPPCPAPELLGGPSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSP |
| 136 | Fc monomer-5 | DKTHTCPPCPAPELLGGPSVFLFPPKPKDTLMISRTPEVTCVVV |
| | | DVSHEDPEVKFNWYVDGVEVHNAKTKPREEQYGSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGK |
| 137 | Fc monomer-6 | DKTHTCPPCPAPELLGGPSVFLFPPKPKDTLMISRTPEVTCVVV |
| | | DVSHEDPEVKFNWYVDGVEVHNAKTKPREEQYGSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSP |
| 138 | Fc monomer-7 | DKTHTCPPCPAPELLGGPSVFLFPPKPKDTLMISRTPEVTCVVV |
| | | DVSHEDPEVKFNWYVDGVEVHNAKTKPCEEQYNSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGK |
| 139 | Fc monomer-8 | DKTHTCPPCPAPELLGGPSVFLFPPKPKDTLMISRTPEVTCVVV |
| | | DVSHEDPEVKFNWYVDGVEVHNAKTKPCEEQYNSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSP |
| 140 | scFc-1 | DKTHTCPPCPAPELLGGPSVFLFPPKPKDTLMISRTPEVTCVVV |
| | | DVSHEDPEVKFNWYVDGVEVHNAKTKPCEEQYGSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGKGGGGSGGGGSGGGGSGGGGSGGGG |
| | | SGGGGSDKTHTCPPCPAPELLGGPSVFLFPPKPKDTLMISRTPE |
| | | VTCVVVDVSHEDPEVKFNWYVDGVEVHNAKTKPCEEQYGST |
| | | YRCVSVLTVLHQDWLNGKEYKCKVSNKALPAPIEKTISKAKG |
| | | QPREPQVYTLPPSREEMTKNQVSLTCLVKGFYPSDIAVEWES |
| | | NGQPENNYKTTPPVLDSDGSFFLYSKLTVDKSRWQQGNVFSC |
| | | SVMHEALHNHYTQKSLSLSPGK |
| 141 | scFc-2 | DKTHTCPPCPAPELLGGPSVFLFPPKPKDTLMISRTPEVTCVVV |
| | | DVSHEDPEVKFNWYVDGVEVHNAKTKPCEEQYGSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGGGGSGGGGSGGGGSGGGGSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPCEEQYGSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSP |
| 142 | scFc-3 | DKTHTCPPCPAPELLGGPSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGKGGGGSGGGGSGGGGSGGGGSGGGG |
| | | SGGGGSDKTHTCPPCPAPELLGGPSVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPAPIEKTISKAKG |
| | | QPREPQVYTLPPSREEMTKNQVSLTCLVKGFYPSDIAVEWES |
| | | NGQPENNYKTTPPVLDSDGSFFLYSKLTVDKSRWQQGNVFSC |
| | | SVMHEALHNHYTQKSLSLSPGK |
| 143 | scFc-4 | DKTHTCPPCPAPELLGGPSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGGGGSGGGGSGGGGSGGGGSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYNSTY |
| | | RVVSVLTVLHQDWLNGKEYKCKVSNKALPAPIEKTISKAKGQ |
| | | PREPQVYTLPPSREEMTKNQVSLTCLVKGFYPSDIA VEWESN |
| | | GQPENNYKTTPPVLDSDGSFFLYSKLTVDKSRWQQGNVFSCS |
| | | VMHEALHNHYTQKSLSLSP |
| 144 | scFc-5 | DKTHTCPPCPAPELLGGPSVFLFPPKPKDTLMISRTPEVTCVVV |
| | | DVSHEDPEVKFNWYVDGVEVHNAKTKPREEQYGSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGKGGGGSGGGGSGGGGSGGGGSGGGG |
| | | SGGGGSDKTHTCPPCPAPELLGGPSVFLFPPKPKDTLMISRTPE |
| | | VTCVVVDVSHEDPEVKFNWYVDGVEVHNAKTKPREEQYGST |
| | | YRVVSVLTVLHQDWLNGKEYKCKVSNKALPAPIEKTISKAKG |
| | | QPREPQVYTLPPSREEMTKNQVSLTCLVKGFYPSDIAVEWES |
| | | NGQPENNYKTTPPVLDSDGSFFLYSKLTVDKSRWQQGNVFSC |
| | | SVMHEALHNHYTQKSLSLSPGK |
| 145 | scFc-6 | DKTHTCPPCPAPELLGGPSVFLFPPKPKDTLMISRTPEVTCVVV |
| | | DVSHEDPEVKFNWYVDGVEVHNAKTKPREEQYGSTYRVVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGGGGSGGGGSGGGGSGGGGSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPREEQYGSTY |
| | | RVVSVLTVLHQDWLNGKEYKCKVSNKALPAPIEKTISKAKGQ |
| | | PREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSP |
| 146 | scFc-7 | DKTHTCPPCPAPELLGGPSVFLFPPKPKDTLMISRTPEVTCVVV |
| | | DVSHEDPEVKFNWYVDGVEVHNAKTKPCEEQYNSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGKGGGGSGGGGSGGGGSGGGGSGGGG |
| | | SGGGGSDKTHTCPPCPAPELLGGPSVFLFPPKPKDTLMISRTPE |
| | | VTCVVVDVSHEDPEVKFNWYVDGVEVHNAKTKPCEEQYNST |
| | | YRCVSVLTVLHQDWLNGKEYKCKVSNKALPAPIEKTISKAKG |
| | | QPREPQVYTLPPSREEMTKNQVSLTCLVKGFYPSDIAVEWES |
| | | NGQPENNYKTTPPVLDSDGSFFLYSKLTVDKSRWQQGNVFSC |
| | | SVMHEALHNHYTQKSLSLSPGK |
| 147 | scFc-8 | DKTHTCPPCPAPELLGGPSVFLFPPKPKDTLMISRTPEVTCVVV |
| | | DVSHEDPEVKFNWYVDGVEVHNAKTKPCEEQYNSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGGGGSGGGGSGGGGSGGGGSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPCEEQYNSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSP |
| 148 | scFc-9 | DKTHTCPPCPAPELLGGPSVFLFPPKPKDTLMISRTPEVTCVVV |
| | | DVSHEDPEVKFNWYVDGVEVHNAKTKPCEEQYGSTYRCVSV |
| | | LTVLHQDWLNGKEYKCKVSNKALPAPIEKTISKAKGQPREPQ |
| | | VYTLPPSREEMTKNQVSLTCLVKGFYPSDIAVEWESNGQPEN |
| | | NYKTTPPVLDSDGSFFLYSKLTVDKSRWQQGNVFSCSVMHE |
| | | ALHNHYTQKSLSLSPGGGGSGGGGSGGGGSGGGGSGGGGSG |
| | | GGGSDKTHTCPPCPAPELLGGPSVFLFPPKPKDTLMISRTPEVT |
| | | CVVVDVSHEDPEVKFNWYVDGVEVHNAKTKPCEEQYGSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGK |
| 149 | Anti-CLDN18.2 | GYYMH |
| | CDRH1 | |
| 150 | Anti-CLDN18.2 | WINPNSGGTKYAQKFQG |
| | CDRH2 | |
| 151 | Anti-CLDN18.2 | DRITVAGTYYYYGMDV |
| | CDRH3 | |
| 152 | Anti-CLDN18.2 VH | QVQLVQSGAEVKKPGASVKVSCKASGYTFTGYYMHWVRQA |
| | | PGQCLEWMGWINPNSGGTKYAQKFQGRVTMTRDTSISTAYM |
| | | ELSRLRSDDTAVYYCARDRITVAGTYYYYGMDVWGQGTTVT |
| | | VSS |
| 153 | Anti-CLDN18.2 | QVQMVQSGAEVKKHGASVKVSCKASGYTFTGYYMHWVRQ |
| | VH.2 | APGQCLEWMGWINPNSGGTKYAQKFQGRVTMTRDTSISTAY |
| | | MELSRLRSDDTAVYYCARDRITVAGTYYYYGMDVWGQGTT |
| | | VTVSS |
| 154 | Anti-CLDN18.2 | RASQGVNNWLA |
| | CDRL1 | |
| 155 | Anti-CLDN18.2 | TASSLQS |
| | CDRL2 | |
| 156 | Anti-CLDN18.2 | QQANSFPIT |
| | CDRL3 | |
| 157 | Anti-CLDN18.2 VL | DIQMTQSPSSVSASVGDRVTITCRASQGVNNWLAWYQQKPG |
| | | KAPKLLIYTASSLQSGVPSRFSGSGSGTDFTLTIRSLQPEDFAT |
| | | YYCQQANSFPITFGCGTRLEIK |
| 158 | Anti-CLDN18.2 | QVQLVQSGAEVKKPGASVKVSCKASGYTFTGYYMHWVRQA |
| | scFv | PGQCLEWMGWINPNSGGTKYAQKFQGRVTMTRDTSISTAYM |
| | | ELSRLRSDDTAVYYCARDRITVAGTYYYYGMDVWGQGTTVT |
| | | VSSGGGGSGGGGSGGGGSDIQMTQSPSSVSASVGDRVTITCR |
| | | ASQGVNNWLAWYQQKPGKAPKLLIYTASSLQSGVPSRFSGSG |
| | | SGTDFTLTIRSLQPEDFATYYCQQANSFPITFGCGTRLEIK |
| 159 | Anti-CLDN18.2 | QVQMVQSGAEVKKHGASVKVSCKASGYTFTGYYMHWVRQ |
| | scFv.2 | APGQCLEWMGWINPNSGGTKYAQKFQGRVTMTRDTSISTAY |
| | | MELSRLRSDDTAVYYCARDRITVAGTYYYYGMDVWGQGTT |
| | | VTVSSGGGGSGGGGSGGGGSDIQMTQSPSSVSASVGDRVTIT |
| | | CRASQGVNNWLAWYQQKPGKAPKLLIYTASSLQSGVPSRFS |
| | | GSGSGTDFTLTIRSLQPEDFATYYCQQANSFPITFGCGTRLEIK |
| 160 | CLDN18.2 x CD3 | QVQLVQSGAEVKKPGASVKVSCKASGYTFTGYYMHWVRQA |
| | scFc | PGQCLEWMGWINPNSGGTKYAQKFQGRVTMTRDTSISTAYM |
| | | ELSRLRSDDTAVYYCARDRITVAGTYYYYGMDVWGQGTTVT |
| | | VSSGGGGSGGGGSGGGGSDIQMTQSPSSVSASVGDRVTITCR |
| | | ASQGVNNWLAWYQQKPGKAPKLLIYTASSLQSGVPSRFSGSG |
| | | SGTDFTLTIRSLQPEDFATYYCQQANSFPITFGCGTRLEIKSGG |
| | | GGSEVLVESGGGLVQPGGSLKLSCAASGFTFNKYAMNWVR |
| | | QAPGKGLEWVARIRSKYNNYATYYADSVKDRFTISRDDSKN |
| | | TAYLQMNNLKTEDTAVYYCVRHGNFGNSYISYWAYWGQGT |
| | | LVTVSSGGGGSGGGGSGGGGSQTVVTQEPSLTVSPGGTVTLT |
| | | CGSSTGAVTSGNYPNWVQQKPGQAPRGLIGGTKFLAPGTPAR |
| | | FSGSLLGGKAALTLSGVQPEDEAEYYCVLWYSNRWVFGGGT |
| | | KLTVLGGGGDKTHTCPPCPAPELLGGPSVFLFPPKPKDTLMIS |
| | | RTPEVTCVVVDVSHEDPEVKFNWYVDGVEVHNAKTKPCEEQ |
| | | YGSTYRCVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGKGGGGSGGGGSGGGG |
| | | SGGGGSGGGGSGGGGSDKTHTCPPCPAPELLGGPSVFLFPPKP |
| | | KDTLMISRTPEVTCVVVDVSHEDPEVKFNWYVDGVEVHNAK |
| | | TKPCEEQYGSTYRCVSVLTVLHQDWLNGKEYKCKVSNKALP |
| | | APIEKTISKAKGQPREPQVYTLPPSREEMTKNQVSLTCLVKGF |
| | | YPSDIAVEWESNGQPENNYKTTPPVLDSDGSFFLYSKLTVDKS |
| | | RWQQGNVFSCSVMHEALHNHYTQKSLSLSPGK |
| 161 | CLDN18.2 x CD3 | QVQMVQSGAEVKKHGASVKVSCKASGYTFTGYYMHWVRQ |
| | scFc.2 | APGQCLEWMGWINPNSGGTKYAQKFQGRVTMTRDTSISTAY |
| | | MELSRLRSDDTAVYYCARDRITVAGTYYYYGMDVWGQGTT |
| | | VTVSSGGGGSGGGGSGGGGSDIQMTQSPSSVSASVGDRVTIT |
| | | CRASQGVNNWLAWYQQKPGKAPKLLIYTASSLQSGVPSRFS |
| | | GSGSGTDFTLTIRSLQPEDFATYYCQQANSFPITFGCGTRLEIK |
| | | SGGGGSEVQLVESGGGLVQPGGSLKLSCAASGFTFNKYAMN |
| | | WVRQAPGKGLEWVARIRSKYNNYATYYADSVKDRFTISRDD |
| | | SKNTAYLQMNNLKTEDTAVYYCVRHGNFGNSYISYWAYWG |
| | | QGTLVTVSSGGGGSGGGGSGGGGSQTVVTQEPSLTVSPGGTV |
| | | TLTCGSSTGAVTSGNYPNWVQQKPGQAPRGLIGGTKFLAPGT |
| | | PARFSGSLLGGKAALTLSGVQPEDEAEYYCVLWYSNRWVFG |
| | | GGTKLTVLGGGGDKTHTCPPCPAPELLGGPSVFLFPPKPKDTL |
| | | MISRTPEVTCVVVDVSHEDPEVKFNWYVDGVEVHNAKTKPC |
| | | EEQYGSTYRCVSVLTVLHQDWLNGKEYKCKVSNKALPAPIE |
| | | KTISKAKGQPREPQVYTLPPSREEMTKNQVSLTCLVKGFYPSD |
| | | IAVEWESNGQPENNYKTTPPVLDSDGSFFLYSKLTVDKSRWQ |
| | | QGNVFSCSVMHEALHNHYTQKSLSLSPGKGGGGSGGGGSGG |
| | | GGSGGGGSGGGGSGGGGSDKTHTCPPCPAPELLGGPSVFLFPP |
| | | KPKDTLMISRTPEVTCVVVDVSHEDPEVKFNWYVDGVEVHN |
| | | AKTKPCEEQYGSTYRCVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 162 | Anti-MUC17 | GYYWS |
| | CDRH1 | |
| 163 | Anti-MUC17 | DIDASGSTKYNPSLKS |
| | CDRH2 | |
| 164 | Anti-MUC17 | KKYSTVWSYFDN |
| | CDRH3 | |
| 165 | Anti-MUC17 VH | QVQLQQWGAGLLKPSETLSLTCAVYGGSFSGYYWSWIRQPP |
| | | GKCLEWIGDIDASGSTKYNPSLKSRVTISLDTSKNQFSLKLNS |
| | | VTAADTAVYFCARKKYSTVWSYFDNWGQGTLVTVSS |
| 166 | Anti-MUC17 | SGDKLGDKYAS |
| | CDRL1 | |
| 167 | Anti-MUC17 | QDRKRPS |
| | CDRL2 | |
| 168 | Anti-MUC17 | QAWGSSTAV |
| | CDRL3 | |
| 169 | Anti-MUC17 VL | SYELTQPSSVSVPPGQTASITCSGDKLGDKYASWYQQKPGQS |
| | | PVLVIYQDRKRPSGVPERFSGSNSGNTATLTISGTQAMDEADY |
| | | YCQAWGSSTAVFGCGTKLTVL |
| 170 | Anti-MUC17 scFv | QVQLQQWGAGLLKPSETLSLTCAVYGGSFSGYYWSWIRQPP |
| | | GKCLEWIGDIDASGSTKYNPSLKSRVTISLDTSKNQFSLKLNS |
| | | VTAADTAVYFCARKKYSTVWSYFDNWGQGTLVTVSSGGGG |
| | | SGGGGSGGGGSSYELTQPSSVSVPPGQTASITCSGDKLGDKYA |
| | | SWYQQKPGQSPVLVIYQDRKRPSGVPERFSGSNSGNTATLTIS |
| | | GTQAMDEADYYCQAWGSSTAVFGCGTKLTVL |
| 171 | MUC17 x CD3 scFc | QVQLQQWGAGLLKPSETLSLTCAVYGGSFSGYYWSWIRQPP |
| | | GKCLEWIGDIDASGSTKYNPSLKSRVTISLDTSKNQFSLKLNS |
| | | VTAADTAVYFCARKKYSTVWSYFDNWGQGTLVTVSSGGGG |
| | | SGGGGSGGGGSSYELTQPSSVSVPPGQTASITCSGDKLGDKYA |
| | | SWYQQKPGQSPVLVIYQDRKRPSGVPERFSGSNSGNTATLTIS |
| | | GTQAMDEADYYCQAWGSSTAVFGCGTKLTVLSGGGGSEVQ |
| | | LVESGGGLVQPGGSLKLSCAASGFTFNKYAMNWVRQAPGKG |
| | | LEWVARIRSKYNNYATYYADSVKDRFTISRDDSKNTAYLQM |
| | | NNLKTEDTAVYYCVRHGNFGNSYISYWAYWGQGTLVTVSSG |
| | | GGGSGGGGSGGGGSQTVVTQEPSLTVSPGGTVTLTCGSSTGA |
| | | VTSGNYPNWVQQKPGQAPRGLIGGTKFLAPGTPARFSGSLLG |
| | | GKAALTLSGVQPEDEAEYYCVLWYSNRWVFGGGTKLTVLG |
| | | GGGDKTHTCPPCPAPELLGGPSVFLFPPKPKDTLMISRTPEVT |
| | | CVVVDVSHEDPEVKFNWYVDGVEVHNAKTKPCEEQYGSTY |
| | | RCVSVLTVLHQDWLNGKEYKCKVSNKALPAPIEKTISKAKGQ |
| | | PREPQVYTLPPSREEMTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGKGGGGSGGGGSGGGGSGGGGS |
| | | GGGGSGGGGSDKTHTCPPCPAPELLGGPSVFLFPPKPKDTLMI |
| | | SRTPEVTCVVVDVSHEDPEVKFNWYVDGVEVHNAKTKPCEE |
| | | QYGSTYRCVSVLTVLHQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |

What is claimed:

1. A method for administering a therapeutic dose of a bispecific T-cell engaging molecule to a patient diagnosed with cancer, comprising administering to the patient an initiation cycle of the bispecific T-cell engaging molecule, said initiation cycle comprising:
administering a priming dose of the bispecific T-cell engaging molecule by continuous intravenous infusion over a period of 1 day to 7 days; and
administering after the priming dose a therapeutic dose of the bispecific T-cell engaging molecule by a bolus intravenous infusion,
wherein the bispecific T-cell engaging molecule comprises a first domain that specifically binds to a target cancer cell antigen, a second domain that specifically binds to human CD3, and an Fc domain.

2. The method of claim 1, wherein the therapeutic dose of the bispecific T-cell engaging molecule is administered once every 7 days for the duration of the initiation cycle.

3. The method of claim 1, wherein the therapeutic dose of the bispecific T-cell engaging molecule is administered once every 14 days for the duration of the initiation cycle.

4. The method of any one of claims 1 to 3, wherein the priming dose of the bispecific T-cell engaging molecule is administered over a period of about 2 days.

5. The method of any one of claims 1 to 3, wherein the priming dose of the bispecific T-cell engaging molecule is administered over a period of about 3 days.

6. The method of any one of claims 1 to 3, wherein the priming dose of the bispecific T-cell engaging molecule is administered over a period of about 4 days.

7. The method of any one of claims 1 to 3, wherein the priming dose of the bispecific T-cell engaging molecule is administered over a period of about 5 days.

8. The method of any one of claims 1 to 3, wherein the priming dose of the bispecific T-cell engaging molecule is administered over a period of about 7 days.

9. The method of any one of claims 1 to 8, wherein the therapeutic dose is administered on the same day the continuous intravenous infusion of the priming dose ends.

10. The method of any one of claims 1 to 8, wherein the therapeutic dose is administered about 1 day to about 7 days after the priming dose.

11. The method of claim 10, wherein the therapeutic dose is administered about 1 day after the priming dose.

12. The method of claim 10, wherein the therapeutic dose is administered about 3 days after the priming dose.

13. The method of claim 10, wherein the therapeutic dose is administered about 4 days after the priming dose.

14. The method of claim 10, wherein the therapeutic dose is administered about 5 days after the priming dose.

15. The method of claim 10, wherein the therapeutic dose is administered about 6 days after the priming dose.

16. The method of any one of claims 1 to 15, further comprising administering a boost dose of the bispecific T-cell engaging molecule by a bolus intravenous infusion after the priming dose and before the therapeutic dose.

17. The method of claim 16, wherein the boost dose is about 30% to about 40% of the priming dose.

18. The method of any one of claims 1 to 17, wherein the duration of the initiation cycle is about 28 days.

19. The method of claim 18, wherein the priming dose of the bispecific T-cell engaging molecule is administered over days 1 to 3 of the initiation cycle and the therapeutic dose of the bispecific T-cell engaging molecule is administered on days 8 and 22 of the initiation cycle.

20. The method of claim 18, wherein the priming dose of the bispecific T-cell engaging molecule is administered over days 1 to 2 of the initiation cycle and the therapeutic dose of the bispecific T-cell engaging molecule is administered on days 8, 15, and 22 of the initiation cycle.

21. The method of claim 20, further comprising administering a boost dose of the bispecific T-cell engaging molecule by bolus intravenous infusion on day 3 of the initiation cycle.

22. The method of claim 18, wherein the priming dose of the bispecific T-cell engaging molecule is administered over days 1 to 7 of the initiation cycle and the therapeutic dose of the bispecific T-cell engaging molecule is administered on days 8, 15, and 22 of the initiation cycle.

23. The method of claim 18, wherein the priming dose of the bispecific T-cell engaging molecule is administered over days 1 to 4 of the initiation cycle and the therapeutic dose of the bispecific T-cell engaging molecule is administered on days 8, 15, and 22 of the initiation cycle.

24. The method of any one of claims 1 to 23, wherein the priming dose is about 10% to about 80% of the therapeutic dose.

25. The method of any one of claims 1 to 23, wherein the priming dose is about 15% to about 50% of the therapeutic dose.

26. The method of any one of claims 1 to 25, further comprising administering to the patient a maintenance cycle of the bispecific T-cell engaging molecule, wherein the maintenance cycle comprises administering the therapeutic dose of the bispecific T-cell engaging molecule by a bolus intravenous infusion once every 7 days or once every 14 days.

27. The method of claim 26, wherein the duration of the maintenance cycle is about 28 days.

28. The method of claim 26 or 27, wherein the maintenance cycle is administered the following day after completing the initiation cycle.

29. The method of claim 26 or 27, wherein the maintenance cycle is administered about 7 days following completion of the initiation cycle.

30. The method of any one of claims 26 to 29, wherein two or more maintenance cycles are administered to the patient.

31. The method of any one of claims 1 to 30, wherein the first domain of the bispecific T-cell engaging molecule specifically binds to a target cancer cell antigen selected from MUC17, CLDN18.2, CD19, CD33, FLT3, DLL3, BCMA and PSMA.

32. The method of any one of claims 1 to 31, wherein the bispecific T-cell engaging molecule comprises, in an amino to carboxyl order:
(i) the first domain that specifically binds to the target cancer cell antigen comprising a first immunoglobulin heavy chain variable region (VH1) and a first immunoglobulin light chain variable region (VL1);
(ii) the second domain that specifically binds to human CD3 comprising a second immunoglobulin heavy chain variable region (VH2), and a second immunoglobulin light chain variable region (VL2); and
(iii) the Fc domain comprising two Fc monomers, each monomer comprising an immunoglobulin hinge region, a CH2 domain, and a CH3 domain, wherein said two monomers are fused to each other via a peptide linker.

33. The method of claim 31 or 32, wherein the first domain of the bispecific T-cell engaging molecule specifically binds to PSMA and the patient is diagnosed with prostate cancer.

34. The method of claim 33, wherein the first domain comprises a VH1 comprising a CDRH1 having the sequence of SEQ ID NO: 51, a CDRH2 having the sequence of SEQ ID NO: 52, and a CDRH3 having the sequence of SEQ ID NO: 53, and a VL1 comprising a CDRL1 having the sequence of SEQ ID NO: 55, a CDRL2 having the sequence of SEQ ID NO: 56, and a CDRL3 having the sequence of SEQ ID NO: 57; and
wherein the second domain comprises a VH2 comprising a CDRH1 having the sequence of SEQ ID NO: 65, a CDRH2 having the sequence of SEQ ID NO: 66, and a CDRH3 having the sequence of SEQ ID NO: 67, and a VL2 comprising a CDRL1 having the sequence of SEQ ID NO: 87, a CDRL2 having the sequence of SEQ ID NO: 83, and a CDRL3 having the sequence of SEQ ID NO: 88.

35. The method of claim 34, wherein VH1 comprises the sequence of SEQ ID NO: 54, VL1 comprises the sequence of SEQ ID NO: 58, VH2 comprises the sequence of SEQ ID NO: 90, and VL2 comprises the sequence of SEQ ID NO: 100.

36. The method of any one of claims 32 to 35, wherein the bispecific T-cell engaging molecule is a single chain polypeptide comprising the sequence of SEQ ID NO: 60.

37. The method of any one of claims 33 to 36, wherein the initiation cycle comprises:
administering a priming dose of about 30 μg to about 150 μg of the bispecific T-cell engaging molecule over a period of about 3 days; and
administering a therapeutic dose of about 300 μg to about 600 μg of the bispecific T-cell engaging molecule, wherein the therapeutic dose is administered about five days after administration of the priming dose.

38. The method of claim 37, wherein the initiation cycle comprises:
administering a priming dose of about 90 μg of the bispecific T-cell engaging molecule over a period of about 3 days; and
administering a therapeutic dose of about 300 μg of the bispecific T-cell engaging molecule, wherein the therapeutic dose is administered about five days after administration of the priming dose.

39. The method of claim 37 or 38, further comprising administering to the patient a maintenance cycle of the bispecific T-cell engaging molecule, wherein the maintenance cycle comprises administering the therapeutic dose of the bispecific T-cell engaging molecule by a bolus intravenous infusion once every 14 days.

40. The method of claim 31 or 32, wherein the first domain of the bispecific T-cell engaging molecule specifically binds to BCMA and the patient is diagnosed with multiple myeloma.

41. The method of claim 40, wherein the first domain comprises a VH1 comprising a CDRH1 having the sequence of SEQ ID NO: 41, a CDRH2 having the sequence of SEQ ID NO: 42, and a CDRH3 having the sequence of SEQ ID NO: 43, and a VL1 comprising a CDRL1 having the sequence of SEQ ID NO: 45, a CDRL2 having the sequence of SEQ ID NO: 46, and a CDRL3 having the sequence of SEQ ID NO: 47; and
wherein the second domain comprises a VH2 comprising a CDRH1 having the sequence of SEQ ID NO: 65, a CDRH2 having the sequence of SEQ ID NO: 66, and a CDRH3 having the sequence of SEQ ID NO: 67, and a VL2 comprising a CDRL1 having the sequence of SEQ ID NO: 87, a CDRL2 having the sequence of SEQ ID NO: 83, and a CDRL3 having the sequence of SEQ ID NO: 88.

42. The method of claim 41, wherein VH1 comprises the sequence of SEQ ID NO: 44, VL1 comprises the sequence of SEQ ID NO: 48, VH2 comprises the sequence of SEQ ID NO: 90, and VL2 comprises the sequence of SEQ ID NO: 100.

43. The method of any one of claims 40 to 42, wherein the bispecific T-cell engaging molecule is a single chain polypeptide comprising the sequence of SEQ ID NO: 50.

44. The method of any one of claims 40 to 43, wherein the initiation cycle comprises:
administering a priming dose of about 8,400 μg to about 16,100 μg of the bispecific T-cell engaging molecule over a period of about 7 days; and
administering a therapeutic dose of about 12,000 μg to about 19,500 μg of the bispecific T-cell engaging molecule, wherein the therapeutic dose is administered about one day after administration of the priming dose.

45. The method of any one of claims 40 to 43, wherein the initiation cycle comprises:
administering a priming dose of about 4,600 μg to about 9,200 μg of the bispecific T-cell engaging molecule over a period of about 2 days; and
administering a therapeutic dose of about 12,000 μg to about 19,500 μg of the bispecific T-cell engaging molecule, wherein the therapeutic dose is administered about six days after administration of the priming dose.

46. The method of claim 45, further comprising administering a boost dose of about 800 μg to about 1,600 μg of the bispecific T-cell engaging molecule by a bolus intravenous infusion about one day after the priming dose and about five days before the therapeutic dose.

47. The method of any one of claims 44 to 46, further comprising administering to the patient a maintenance cycle of the bispecific T-cell engaging molecule, wherein the maintenance cycle comprises administering the therapeutic dose of the bispecific T-cell engaging molecule by a bolus intravenous infusion once every 7 days.

48. The method of any one of claims 32 to 46, wherein each of said Fc monomers of the Fc domain comprises the sequence of SEQ ID NO: 132.

49. The method of any one of claims 32 to 48, wherein the Fc domain comprises the sequence of SEQ ID NO: 140.

50. The method of any one of claims 1 to 49, wherein the continuous intravenous infusion delivers the priming dose at a constant rate.

51. The method of any one of claims 1 to 50, wherein the bolus intravenous infusion is an infusion of about 30 min to about 90 min.

52. The method of claim 51, wherein the bolus intravenous infusion is an infusion of about 60 min.

53. A bispecific T-cell engaging molecule that specifically binds to a target cancer cell antigen and human CD3 for use in a method for treating cancer in a patient in need thereof, wherein the method comprises administering to the patient an initiation cycle of the bispecific T-cell engaging molecule, said initiation cycle comprising:
administering a priming dose of the bispecific T-cell engaging molecule by continuous intravenous infusion over a period of 1 day to 7 days; and
administering after the priming dose a therapeutic dose of the bispecific T-cell engaging molecule by a bolus intravenous infusion,
wherein the bispecific T-cell engaging molecule comprises a first domain that specifically binds to a target cancer cell antigen, a second domain that specifically binds to human CD3, and an Fc domain.

54. The bispecific T-cell engaging molecule for use according to claim 53, wherein the therapeutic dose of the bispecific T-cell engaging molecule is administered once every 7 days for the duration of the initiation cycle.

55. The bispecific T-cell engaging molecule for use according to claim 53, wherein the therapeutic dose of the bispecific T-cell engaging molecule is administered once every 14 days for the duration of the initiation cycle.

56. The bispecific T-cell engaging molecule for use according to any one of claims 53 to 55, wherein the priming dose of the bispecific T-cell engaging molecule is administered over a period of about 2 days.

57. The bispecific T-cell engaging molecule for use according to any one of claims 53 to 55, wherein the priming dose of the bispecific T-cell engaging molecule is administered over a period of about 3 days.

58. The bispecific T-cell engaging molecule for use according to any one of claims 53 to 55, wherein the priming dose of the bispecific T-cell engaging molecule is administered over a period of about 4 days.

59. The bispecific T-cell engaging molecule for use according to any one of claims 53 to 55, wherein the priming dose of the bispecific T-cell engaging molecule is administered over a period of about 5 days.

60. The bispecific T-cell engaging molecule for use according to any one of claims 53 to 55, wherein the priming dose of the bispecific T-cell engaging molecule is administered over a period of about 7 days.

61. The bispecific T-cell engaging molecule for use according to any one of claims 53 to 60, wherein the therapeutic dose is administered on the same day the continuous intravenous infusion of the priming dose ends.

62. The bispecific T-cell engaging molecule for use according to any one of claims 53 to 60, wherein the therapeutic dose is administered about 1 day to about 7 days after the priming dose.

63. The bispecific T-cell engaging molecule for use according to claim 62, wherein the therapeutic dose is administered about 1 day after the priming dose.

64. The bispecific T-cell engaging molecule for use according to claim 62, wherein the therapeutic dose is administered about 3 days after the priming dose.

65. The bispecific T-cell engaging molecule for use according to claim 62, wherein the therapeutic dose is administered about 4 days after the priming dose.

66. The bispecific T-cell engaging molecule for use according to claim 62, wherein the therapeutic dose is administered about 5 days after the priming dose.

67. The bispecific T-cell engaging molecule for use according to claim 62, wherein the therapeutic dose is administered about 6 days after the priming dose.

68. The bispecific T-cell engaging molecule for use according to any one of claims 53 to 67, wherein the method further comprises administering a boost dose of the bispecific T-cell engaging molecule by a bolus intravenous infusion after the priming dose and before the therapeutic dose.

69. The bispecific T-cell engaging molecule for use according to claim 68, wherein the boost dose is about 30% to about 40% of the priming dose.

70. The bispecific T-cell engaging molecule for use according to any one of claims 53 to 69, wherein the duration of the initiation cycle is about 28 days.

71. The bispecific T-cell engaging molecule for use according to claim 70, wherein the priming dose of the bispecific T-cell engaging molecule is administered over days 1 to 3 of the initiation cycle and the therapeutic dose of the bispecific T-cell engaging molecule is administered on days 8 and 22 of the initiation cycle.

72. The bispecific T-cell engaging molecule for use according to claim 70, wherein the priming dose of the bispecific T-cell engaging molecule is administered over days 1 to 2 of the initiation cycle and the therapeutic dose of the bispecific T-cell engaging molecule is administered on days 8, 15, and 22 of the initiation cycle.

73. The bispecific T-cell engaging molecule for use according to claim 72, wherein the method further comprises administering a boost dose of the bispecific T-cell engaging molecule by bolus intravenous infusion on day 3 of the initiation cycle.

74. The bispecific T-cell engaging molecule for use according to claim 70, wherein the priming dose of the bispecific T-cell engaging molecule is administered over days 1 to 7 of the initiation cycle and the therapeutic dose of the bispecific T-cell engaging molecule is administered on days 8, 15, and 22 of the initiation cycle.

75. The bispecific T-cell engaging molecule for use according to claim 70, wherein the priming dose of the bispecific T-cell engaging molecule is administered over days 1 to 4 of the initiation cycle and the therapeutic dose of the bispecific T-cell engaging molecule is administered on days 8, 15, and 22 of the initiation cycle.

76. The bispecific T-cell engaging molecule for use according to any one of claims 53 to 75, wherein the priming dose is about 10% to about 80% of the therapeutic dose.

77. The bispecific T-cell engaging molecule for use according to any one of claims 53 to 75, wherein the priming dose is about 15% to about 50% of the therapeutic dose.

78. The bispecific T-cell engaging molecule for use according to any one of claims 53 to 77, wherein the method further comprises administering to the patient a maintenance cycle of the bispecific T-cell engaging molecule, wherein the maintenance cycle comprises administering the therapeutic dose of the bispecific T-cell engaging molecule by a bolus intravenous infusion once every 7 days or once every 14 days.

79. The bispecific T-cell engaging molecule for use according to claim 78, wherein the duration of the maintenance cycle is about 28 days.

80. The bispecific T-cell engaging molecule for use according to claim 78 or 79, wherein the maintenance cycle is administered the following day after completing the initiation cycle.

81. The bispecific T-cell engaging molecule for use according to claim 78 or 79, wherein the maintenance cycle is administered about 7 days following completion of the initiation cycle.

82. The bispecific T-cell engaging molecule for use according to any one of claims 78 to 81, wherein two or more maintenance cycles are administered to the patient.

83. The bispecific T-cell engaging molecule for use according to any one of claims 53 to 82, wherein the first domain of the bispecific T-cell engaging molecule specifically binds to a target cancer cell antigen selected from MUC17, CLDN18.2, CD19, CD33, FLT3, DLL3, BCMA and PSMA.

84. The bispecific T-cell engaging molecule for use according to any one of claims 53 to 83, wherein the bispecific T-cell engaging molecule comprises, in an amino to carboxyl order:
(i) the first domain that specifically binds the target cancer cell antigen comprising a first immunoglobulin heavy chain variable region (VH1) and a first immunoglobulin light chain variable region (VL1);
(ii) the second domain that specifically binds to human CD3 comprising a second immunoglobulin heavy chain variable region (VH2), and a second immunoglobulin light chain variable region (VL2); and
(iii) the Fc domain comprising two Fc monomers, each monomer comprising an immunoglobulin hinge region, a CH2 domain, and a CH3 domain, wherein said two monomers are fused to each other via a peptide linker.

85. The bispecific T-cell engaging molecule for use according to claim 83 or 84, wherein the first domain of the bispecific T-cell engaging molecule specifically binds to PSMA and the patient is diagnosed with prostate cancer.

86. The bispecific T-cell engaging molecule for use according to claim 85, wherein the first domain comprises a VH1 comprising a CDRH1 having the sequence of SEQ ID NO: 51, a CDRH2 having the sequence of SEQ ID NO: 52, and a CDRH3 having the sequence of SEQ ID NO: 53, and a VL1 comprising a CDRL1 having the sequence of SEQ ID NO: 55, a CDRL2 having the sequence of SEQ ID NO: 56, and a CDRL3 having the sequence of SEQ ID NO: 57; and wherein the second domain comprises a VH2 comprising a CDRH1 having the sequence of SEQ ID NO: 65, a CDRH2 having the sequence of SEQ ID NO: 66, and a CDRH3 having the sequence of SEQ ID NO: 67, and a VL2 comprising a CDRL1 having the sequence of SEQ ID NO: 87, a CDRL2 having the sequence of SEQ ID NO: 83, and a CDRL3 having the sequence of SEQ ID NO: 88.

87. The bispecific T-cell engaging molecule for use according to claim 86, wherein VH1 comprises the sequence of SEQ ID NO: 54, VL1 comprises the sequence of SEQ ID NO: 58, VH2 comprises the sequence of SEQ ID NO: 90, and VL2 comprises the sequence of SEQ ID NO: 100.

88. The bispecific T-cell engaging molecule for use according to any one of claims 84 to 87, wherein the bispecific T-cell engaging molecule is a single chain polypeptide comprising the sequence of SEQ ID NO: 60.

89. The bispecific T-cell engaging molecule for use according to any one of claims 85 to 88, wherein the initiation cycle comprises:
administering a priming dose of about 30 μg to about 150 μg of the bispecific T-cell engaging molecule over a period of about 3 days; and
administering a therapeutic dose of about 300 μg to about 600 μg of the bispecific T-cell engaging molecule, wherein the therapeutic dose is administered about five days after administration of the priming dose.

90. The bispecific T-cell engaging molecule for use according to claim 89, wherein the initiation cycle comprises:
administering a priming dose of about 90 μg of the bispecific T-cell engaging molecule over a period of about 3 days; and
administering a therapeutic dose of about 300 μg of the bispecific T-cell engaging molecule, wherein the therapeutic dose is administered about five days after administration of the priming dose.

91. The bispecific T-cell engaging molecule for use according to claim 89 or 90, wherein the method further comprises administering to the patient a maintenance cycle of the bispecific T-cell engaging molecule, wherein the maintenance cycle comprises administering the therapeutic dose of the bispecific T-cell engaging molecule by a bolus intravenous infusion once every 14 days.

92. The bispecific T-cell engaging molecule for use according to claim 83 or 84, wherein the first domain of the bispecific T-cell engaging molecule specifically binds to BCMA and the patient is diagnosed with multiple myeloma.

93. The bispecific T-cell engaging molecule for use according to claim 92, wherein the first domain comprises a VH1 comprising a CDRH1 having the sequence of SEQ ID NO: 41, a CDRH2 having the sequence of SEQ ID NO: 42, and a CDRH3 having the sequence of SEQ ID NO: 43, and a VL1 comprising a CDRL1 having the sequence of SEQ ID NO: 45, a CDRL2 having the sequence of SEQ ID NO: 46, and a CDRL3 having the sequence of SEQ ID NO: 47; and wherein the second domain comprises a VH2 comprising a CDRH1 having the sequence of SEQ ID NO: 65, a CDRH2 having the sequence of SEQ ID NO: 66, and a CDRH3 having the sequence of SEQ ID NO: 67, and a VL2 comprising a CDRL1 having the sequence of SEQ ID NO: 87, a CDRL2 having the sequence of SEQ ID NO: 83, and a CDRL3 having the sequence of SEQ ID NO: 88.

94. The bispecific T-cell engaging molecule for use according to claim 93, wherein VH1 comprises the sequence of SEQ ID NO: 44, VL1 comprises the sequence of SEQ ID NO: 48, VH2 comprises the sequence of SEQ ID NO: 90, and VL2 comprises the sequence of SEQ ID NO: 100.

95. The bispecific T-cell engaging molecule for use according to any one of claims 92 to 94, wherein the bispecific T-cell engaging molecule is a single chain polypeptide comprising the sequence of SEQ ID NO: 50.

96. The bispecific T-cell engaging molecule for use according to any one of claims 92 to 95, wherein the initiation cycle comprises:
administering a priming dose of about 8,400 μg to about 16,100 μg of the bispecific T-cell engaging molecule over a period of about 7 days; and
administering a therapeutic dose of about 12,000 μg to about 19,500 μg of the bispecific T-cell engaging molecule, wherein the therapeutic dose is administered about one day after administration of the priming dose.

97. The bispecific T-cell engaging molecule for use according to any one of claims 92 to 95, wherein the initiation cycle comprises:
administering a priming dose of about 4,600 μg to about 9,200 μg of the bispecific T-cell engaging molecule over a period of about 2 days; and
administering a therapeutic dose of about 12,000 μg to about 19,500 μg of the bispecific T-cell engaging molecule, wherein the therapeutic dose is administered about six days after administration of the priming dose.

98. The bispecific T-cell engaging molecule for use according to claim 97, wherein the method further comprises administering a boost dose of about 800 μg to about 1,600 μg of the bispecific T-cell engaging molecule by a bolus intravenous infusion about one day after the priming dose and about five days before the therapeutic dose.

99. The bispecific T-cell engaging molecule for use according to any one of claims 96 to 98, wherein the method further comprises administering to the patient a maintenance cycle of the bispecific T-cell engaging molecule, wherein the maintenance cycle comprises administering the therapeutic dose of the bispecific T-cell engaging molecule by a bolus intravenous infusion once every 7 days.

100. The bispecific T-cell engaging molecule for use according to any one of claims 84 to 99, wherein each of said Fc monomers of the Fc domain comprises the sequence of SEQ ID NO: 132.

101. The bispecific T-cell engaging molecule for use according to any one of claims 84 to 100, wherein the Fc domain comprises the sequence of SEQ ID NO: 140.

102. The bispecific T-cell engaging molecule for use according to any one of claims 53 to 101, wherein the continuous intravenous infusion delivers the priming dose at a constant rate.

103. The bispecific T-cell engaging molecule for use according to any one of claims 53 to 102, wherein the bolus intravenous infusion is an infusion of about 30 min to about 90 min.

104. The bispecific T-cell engaging molecule for use according to claim 103, wherein the bolus intravenous infusion is an infusion of about 60 min.

105. Use of a bispecific T-cell engaging molecule that specifically binds to a target cancer cell antigen and human CD3 for the manufacture of a medicament for the treatment of cancer in a patient in need thereof, wherein the treatment comprises administering to the patient an initiation cycle of the bispecific T-cell engaging molecule, said initiation cycle comprising:
administering a priming dose of the bispecific T-cell engaging molecule by continuous intravenous infusion over a period of 1 day to 7 days; and
administering after the priming dose a therapeutic dose of the bispecific T-cell engaging molecule by a bolus intravenous infusion,
wherein the bispecific T-cell engaging molecule comprises a first domain that specifically binds to a target cancer cell antigen, a second domain that specifically binds to human CD3, and an Fc domain.
